(19) United States
(12) Patent Application Publication
(10) Pub. No.: US 2025/0129295 A1
(43) Pub. Date: Apr. 24, 2025

(54) MICROWAVE-ASSISTED CATALYTIC PYROLYSIS PROCESS AND REACTOR FOR SELECTIVELY CONVERTING ADDITIVE-CONTAINING PLASTIC ALKENES AND ALKANES

(71) Applicant: University of Delaware, Newark, DE (US)

(72) Inventors: Esun Selvam, Newark, DE (US); Pavel Kots, Philadelphia, PA (US); Weiqi Chen, Brooklyn, NY (US); Dionisios G. Vlachos, Newark, DE (US); Jacqueline Ngu, Elkton, MD (US); Sean Najmi, Newark, DE (US); Brandon Vance, Newark, DE (US)

(73) Assignee: University of Delaware, Newark, DE (US)

(21) Appl. No.: 18/948,419

(22) Filed: Nov. 14, 2024

Related U.S. Application Data
(63) Continuation-in-part of application No. PCT/US2023/027762, filed on Jul. 14, 2023.
(60) Provisional application No. 63/389,065, filed on Jul. 14, 2022.

(51) Int. Cl. C10G 1/02; B01J 6/00; B01J 23/30; B01J 27/182; B01J 29/04; B01J 29/12; B01J 29/76; B01J 37/02; B01J 37/08

(52) U.S. Cl. CPC C10G 1/02; B01J 6/008; B01J 23/30; B01J 27/182; B01J 29/041; B01J 29/126; B01J 29/7615; B01J 37/0201; B01J 37/0236; B01J 37/08; C10G 2300/1003; C10G 2400/20; C10G 2400/22

(57) ABSTRACT

The present disclosure provides a pyrolysis process for converting an additive-containing plastic comprising a polyolefin polymer to an alkene, an alkane, or a mixture thereof. The process comprises contacting the plastic with a catalyst in a one-pot pyrolysis system at a temperature between about 350° C. and about 500° C.; wherein the catalyst comprises a solid acid selected from the group consisting of HY, HZSM-5, and Al-SBA-15. The present disclosure further provides a one-pot pyrolysis system and uses of a solid acid in the pyrolysis process for converting plastic comprising a polyolefin polymer to an alkene.

### CROSS REFERENCE TO RELATED APPLICATIONS

This application is a continuation-in-part application of international patent application No. PCT/US2023/027762, filed Jul. 14, 2023, which claims the benefit of U.S. Provisional Patent Application Ser. No. 63/389,065, filed Jul. 14, 2022, the contents of each of which are herein incorporated by reference.

### STATEMENT AS TO FEDERALLY SPONSORED RESEARCH

This invention was made with government support under grant DE-SC0021166 awarded by the Department of Energy. The government has certain rights in the invention.

### BACKGROUND

Plastic waste's environmental, social, and economic impact is profound. Yet, global plastic production is rising, with an annual production of over 365 million metric tons in 2020. A significant fraction of the plastic waste is in single-use plastics, typically composed of polyolefins (PO) (˜60%), including low-and high-density polyethylene (LDPE, HDPE) and polypropylene (PP). The inadequacy of mechanical recycling to effectively handle these materials has led to a surge in chemical upcycling technologies, including pyrolysis, hydrogenolysis, and hydrocracking. The latter two require high pressure-hydrogen, whose production contributes significantly to CO₂ emissions, and expensive noble-metal catalysts (Pt or Ru), producing a range of alkanes, mainly in the fuel range, whose value is low. Thermal pyrolysis is a feedstock agnostic, ambient pressure process that operates at high temperatures (˜500-700° C.); it is energy-intensive and results in an unselective hydrocarbon pool (C2-C50 range) whose separation is challenging and energy intensive (See Zhao, D., et al., The Chemistry and Kinetics of Polyethylene Pyrolysis: A Process to Produce Fuels and Chemicals, ChemSusChem 13 (7) (2020) 1764-1774).

Catalytic pyrolysis at lower temperatures generates narrower product distributions, such as light olefins (See K. Pyra, et al., Towards a greater olefin share in polypropylene cracking—Amorphous mesoporous aluminosilicate competes with zeolites, Appl. Catal. B 297 (2021); M. Artetxe, et al., Production of Light Olefins from Polyethylene in a Two-Step Process: Pyrolysis in a Conical Spouted Bed and Downstream High-Temperature Thermal Cracking, Ind. Eng. Chem. Res. 51 (43) (2012) 13915-13923; A. Eschenbacher, etc., Maximizing light olefins and aromatics as high value base chemicals via single step catalytic conversion of plastic waste, Chem. Eng. J. 428 (2022), 132087), BTX (benzene, toluene, and xylene, see Y. Uemichi, et al., Conversion of Polyethylene into Gasoline-Range Fuels by Two-Stage Catalytic Degradation Using Silica-Alumina and HZSM-5 Zeolite, Ind. Eng. Chem. Res. 38 (2) (1999) 385-390), or gasoline-diesel fuels, Id. While light olefins (C₂-C₄) have been produced, the selectivity to longer olefins (>C4; valued at $ 1700 Mt⁻¹), chemical industry's building blocks for lubricants, jet fuel, surfactants, plasticizers, polyethylene co-monomers, etc., is usually very low (See Zhao, D., et al., The Chemistry and Kinetics of Polyethylene Pyrolysis: A Process to Produce Fuels and Chemicals, ChemSusChem 13 (7) (2020) 1764-1774); W. E. Garwood, et al., Process for converting olefins to high viscosity index lubricants, in: U.S. Patent (Ed.) Mobil Oil Corporation, New York, N.Y., United States, 1985; P. Das, et al., Value-added products from thermochemical treatments of contaminated e-waste plastics, Chemosphere 269 (2021), 129409; C. Jia, et al., Applications, treatments, and reuse of plastics from electrical and electronic equipment, J. Ind. Eng. Chem. 110 (2022) 84-99; or K. Li, et al., Catalytic pyrolysis of film waste over Co/Ni pillared montmorillonites towards H2 production, Chemosphere 299 (2022), 134440. Larger olefins are typically made from smaller ones, mainly ethylene and from C4s; their direct production from plastic waste can fill in a significant market gap and eliminate additional processing steps but this has seldomly been done so far.

Furthermore, an overlooked challenge in plastics recycling is their very low thermal conductivity (See K. Eiermann, et al., Thermal conductivity of high polymers from −180° C. to 90° C., Journal of Polymer Science 57 (165) (1962) 99-106), resulting in slow heating and hours to days long processing. Long processing implies huge reactors that prevent processing close to the source. Most of the studied processes operate at very high temperature and lead to modest to low medium olefins yields.

Microwaves (MW) can efficiently heat materials rapidly and volumetrically and eliminate associated CO₂ emissions by using renewable electricity (See J. A. Menendez, ettt al., Microwave heating processes involving carbon materials, Fuel Proc, Technol. 91 (1) (2010) 1-8; or A. Malhotra, et al., Temperature Homogeneity under Selective and Localized Microwave Heating in Structured Flow Reactors, Ind. Eng. Chem. Res. 60 (18) (2021) 6835-6847). However, plastics' poor dielectric properties (low tand) make them poor MW susceptors. A common approach to overcome this challenge has been to mix the polymer feed with a MW susceptive dielectric material, such as carbon (See C. Ludlow-Palafox, et al., Microwave-Induced Pyrolysis of Plastic Wastes, Ind. Eng. Chem. Res. 40 (22) (2001) 4749-4756; A. Undri, et al., Efficient disposal of waste polyolefins through microwave assisted pyrolysis, Fuel 116 (2014) 662-671; or X. Zhang, H., et al., Gasoline-range hydrocarbons produced from microwave-induced pyrolysis of low-density polyethylene over ZSM-5, Fuel 144 (2015) 33-42). However, many of these MW-assisted deconstruction approaches have been non-catalytic and performed at high temperatures (500-1000° C.), resulting in broad product distributions (See C. Ludlow-Palafox, et al., Microwave-Induced Pyrolysis of Plastic Wastes, Ind. Eng. Chem. Res. 40 (22) (2001) 4749-4756; or A. Undri, et al., Efficient disposal of waste polyolefins through microwave assisted pyrolysis, Fuel 116 (2014) 662-671). Some catalytic approaches have been reported; however, these typically involve a two-stage setup involving a pyrolysis reactor followed by a catalytic reactor with either one or both reactors heated using MWs (See X. Zhang, H., et al., Gasoline-range hydrocarbons produced from microwave-induced pyrolysis of low-density polyethylene over ZSM-5, Fuel 144 (2015) 33-42; or N. Zhou, et al., A structured catalyst of ZSM-5/SiC foam for chemical recycling of waste plastics via catalytic pyrolysis, Chem. Eng. J. 440 (2022), 135836). More recently, Jie et al. proposed a one-pot setup using an Iron-based dielectric catalyst as a MW susceptor to initiate the catalytic deconstruction process (See X. Jie, et al., Microwave-initiated catalytic deconstruction of plastic waste into hydrogen and high-value carbons, Nat. Catal. 3 (11) (2020) 902-912). However, a common limitation of most processes is the use of powder or particulate MW susceptors, which are prone to temperature inhomogeneities due to the formation of hotspots, especially at high heating rates.

Moreover, additives are essential in finished plastics, acting as antioxidants, ultraviolet (UV) light absorbers, fillers, slip agents, lubricant additives, and stabilizers. They impart important functions and enhance polymer lifetime, performance, and processability. A common plastic may contain 20 or more additives. A recent preliminary report revealed that common additives, such as the primary antioxidant Irganox 3114 (1-3114), deactivate metal-supported catalysts during polyethylene catalytic conversion (See Z. R. Hinton et al., Antioxidant-induced transformations of a metal-acid hydrocracking catalyst in the deconstruction of polyethylene waste. Green Chem. 24, 7332-7339 (2022)). The degree of catalyst poisoning by additives and the underlying mechanisms remain unknown. In addition, some additives are toxic to humans and the environment, and their removal is imperative to enable closed-loop recycling, prevent their leaching into the environment, and avoid their incorporation into recycled products. Techniques like additive extraction via solvents and polymer dissolution/precipitation (See S. Ügdüler, K. M. Van Geem, M. Roosen, E. I. P. Delbeke, S. De Meester, Challenges and opportunities of solvent-based additive extraction methods for plastic recycling. Waste Management 104, 148-182 (2020), also see H. J. Vandenburg et al., Critical Review: Analytical Extraction of Additives From Polymers. Analyst 122, 101R-116R (1997)) could remove additives before catalytic conversion, but these methods remain underdeveloped. These processes are anticipated to incur high recycling costs, significant energy use, and CO₂ emissions due to evaporating large volumes of solvents from small fractions of additives (See A. del Carmen Munguía-López et al., Quantifying the environmental benefits of a solvent-based separation process for multilayer plastic films. Green Chemistry 25, 1611-1625 (2023)). Effective additive extraction demands a precise knowledge of the plastics' composition and a comprehensive library of solvents tailored to each additive, posing significant operational challenges.

Accordingly, there is a need for an improved catalytic technology that is energy-efficient, low-temperature, and easy-operation and therefore substantially reduces the environmental footprint of modern plastics and upcycles plastics into high-value products, i.e. longer alkenes or alkanes.

### SUMMARY DESCRIPTION

The present disclosure provides a pyrolysis process for converting a plastic, including an additive-containing plastic, comprising a polyolefin polymer to an alkene.

In one aspect, the present disclosure provides a pyrolysis process for converting a plastic comprising a polyolefin polymer to an alkene, comprising contacting the plastic with a catalyst in a one-pot pyrolysis system at a temperature between about 350° C. and about 500° C.; wherein the catalyst comprises a solid acid; and wherein the one-pot pyrolysis system comprises a microwave-assisted slurry reactor. In some embodiments, the plastic comprises high-density polyethylene (HDPE), low-density polyethylene (LDPE), polypropylene (PP), polystyrene (PS), or a mixture thereof.

In one aspect, the present disclosure provides a pyrolysis process for converting an additive-containing plastic comprising a polyolefin polymer to an alkene, an alkane, or a mixture thereof, comprising contacting the additive-containing plastic with a catalyst in a one-pot pyrolysis system at a temperature between about 350° C. and about 500° C.; wherein the catalyst comprises a solid acid selected from the group consisting of HY, HZSM-5, and Al-SBA-15; and wherein the one-pot pyrolysis system comprises a microwave-assisted slurry reactor. Due to a synergy of strong acidity, hierarchical porosity, elevated temperatures, and rapid heating, the pyrolysis process described herein provides an efficient way of converting additive-containing plastics to an alkene, an alkane, or a mixture thereof.

In one aspect, the present disclosure further provides a one-pot pyrolysis system comprising a microwave-assisted slurry reactor; and a condenser section; wherein the microwave-assisted slurry reactor comprises a reaction vessel and a microwave source. In some embodiments, the reaction vessel comprises a microwave susceptor.

In one aspect, The present disclosure further provides use of a solid acid in a pyrolysis process for converting plastic comprising a polyolefin polymer to an alkene; wherein the solid acid is selected from the group consisting of P-SiO2, 15WZr, 25WZr, H-ZSM-5, Al-MCM-41, Al-SBA-15, and HY(30).

In one aspect, the present disclosure further provides use of a solid acid in a pyrolysis process for converting an additive-containing plastic to an alkene or an alkane; wherein the solid acid is selected from the group consisting of HY, HZSM-5, and Al-SBA-15.

### BRIEF DESCRIPTION OF THE FIGURES

FIG. 1A shows technology to medium size olefins disclosed in the art and presented in the current disclosure. The conversion of naphtha and steam cracking that is disclosed in the art proceeds in two steps: production of ethylene and its oligomerization to medium size olefins. The technology in the current disclosure achieves this in one step.

FIG. 1B shows a MW slurry reactive distillation pyrolysis reactor to selectively produce alkenes from LDPE.

FIG. 1C is an illustration of gas bubbling through the polymer-catalyst melt containing the MW absorbing SiC monolith.

FIG. 2A shows an experimental setup for MW-assisted pyrolysis of LDPE.

FIG. 2B shows a reactor for conventional pyrolysis of LDPE with heating bands.

FIG. 2C shows a SiC monolith used as an MW susceptor.

FIG. 2D shows a temperature profile obtained from IR camera during MW pyrolysis of LDPE.

FIG. 3 shows a typical temperature profile for the MW pyrolysis of LDPE.

FIG. 4 shows an experimental setup for Conventional Heating Pyrolysis of LDPE.

FIG. 5 shows a GC-MS chromatogram of hydrocarbon-mix calibration standard 1.

FIG. 6 shows a GC-MS chromatogram of hydrocarbon-mix calibration standard 2.

FIG. 7 shows a GC-MS chromatogram of hydrocarbon-mix calibration standard 3.

FIG. 8A-C shows an experimental setup for the temperature profile measurement of MW heating with different filler materials. FIG. 8A shows a schematic of the experimental setup, FIG. 8B shows the SiC monolith and quartz frit before MW heating, and FIG. 8C shows the experimental setup after MW heating with a polymer melt.

FIG. 9 shows a summary of the stages involved in the integrated process for the production of lubricants from LDPE Pyrolysis.

FIG. 10 shows a section of the process for depolymerization and separation of the products.

FIG. 11 shows oligomerization of butenes and propylene to lubricants.

FIG. 12 shows production of lubricants from α-olefins by oligomerization.

FIG. 13 shows system boundaries analysed in the LCA.

FIG. 14A shows comparison of MW pyrolysis with TGA at different temperatures.

FIG. 14B shows effect of changing gas flow rate in MW pyrolysis of LDPE at 375° C. in N2.

FIG. 14C shows comparison of MW pyrolysis performance with H₂ and N₂ at different flow rates.

FIG. 15A shows LDPE conversion in MW pyrolysis over multiple different solid-acid catalysts (Reaction conditions: 375° C., 100 mL min-1 N2 flow, reaction time 200 s).

FIG. 15B shows selectivities of extractable products (based on C-number) in MW pyrolysis over multiple different solid-acid catalysts (Reaction conditions: 375° C., 100 mL min-1 N2 flow, reaction time 200 s).

FIG. 15C shows alkane/olefin selectivities in MW pyrolysis over multiple different solid-acid catalysts (Reaction conditions: 375° C., 100 mL min-1 N2 flow, reaction time 200 s).

FIG. 15D shows typical product distributions using HY(30) in MW pyrolysis (Reaction conditions: 375° C., 100 mL min-1 N2 flow, reaction time 200 s).

FIG. 15E shows typical product distributions using Al-SBA-15 in MW pyrolysis (Reaction conditions: 375° C., 100 mL min-1 N2 flow, reaction time 200 s).

FIG. 16A shows time-dependent LDPE conversion.

FIG. 16B shows major product selectivities over 0.5 Pt-HY.

FIG. 16C shows major product selectivities over Al-SBA-15 catalyst.

FIG. 17A shows performance of MW and CH pyrolysis over HY catalyst, also demonstrating the effect of monolith in CH.

FIG. 17B shows performance of MW and CH pyrolysis over Al-SBA-15 catalyst.

FIG. 17C shows coke in MW and CH pyrolysis.

FIG. 17D shows Raman spectra of coke over 0.5 Pt-HY.

FIGS. 17E-F show Raman spectra in MW and CH pyrolysis, respectively over Al-SBA-15 in N2 flow.

FIG. 17G shows % weight loss in TGA of MW and CH coke obtained from LDPE pyrolysis over 0.5 Pt-HY.

FIG. 17H shows effect of flow rate and size of filler quartz particles between the monolith and the wall on the thermal gradients in MW heating.

FIG. 17I shows effect of Nusselt number on thermal gradients in MW heating predicted by multiscale, multiphysics simulations.

FIG. 18A shows product distribution of extractables obtained from CH Pyrolysis of LDPE over 0.5 Pt-HY(30) (reaction conditions: temperature—375° C., gas flow rate—100 ml min⁻¹ N₂ and time—200 seconds).

FIG. 18B shows product distribution of extractables obtained from MW pyrolysis of LDPE over 0.5 Pt-HY(30) (reaction conditions: temperature —375° C., gas flow rate —100 ml min⁻¹ N₂ and time —200 seconds).

FIG. 19A-D shows reaction pathways in MW-assisted catalytic pyrolysis of LPDE over solid-acid catalysts. (FIG. 19A) Monomolecular cracking. (FIG. 19B) Bimolecular cracking. (FIG. 19C) Coke formation pathways. (FIG. 19D) H-transfer pathway.

FIG. 20A shows comparison of TGA profiles of coked HY catalyst obtained from CH and MW pyrolysis of LDPE in N₂ gas flow.

FIG. 20B shows comparison of DSC profiles of coked HY catalyst obtained from CH and MW pyrolysis of LDPE in N₂ gas flow.

FIG. 21A shows TGA profiles of coked Al-SBA-15 catalyst obtained from CH and MW pyrolysis of LDPE in N₂ gas flow.

FIG. 21B shows DSC profiles of coked Al-SBA-15 catalyst obtained from CH and MW pyrolysis of LDPE in N₂ gas flow.

FIG. 22 shows % weight loss in TGA of MW and CH coke obtained from LDPE pyrolysis over Al-SBA-15.

FIG. 23 shows deconvolution of Raman spectra of coke sample obtained from MW slurry pyrolysis of LDPE over HY catalyst.

FIG. 24 shows Raman spectra of coke in MW pyrolysis of LDPE over Al-SBA-15 in H₂ flow.

FIG. 25 shows temperature profiles by optical fiber and pyrometer for microwave heating with N₂ as filler material.

FIG. 26A-C shows temperature profiles by optical fiber and pyrometer for microwave heating with quartz particle sizes (Fig. A) 30-40 mesh, (Fig. B) 40-60 mesh, and (Fig. C) >60mesh as the filler material.

FIG. 27A-D shows temperature profiles by optical fiber and pyrometer for microwave heating with LDPE as filler material, measured at different N₂ flow rates: (FIG. 27A) 3 mL min⁻¹, (FIG. 27B) 50 mL min⁻¹, (FIG. 27C) 100 mL min⁻¹, and (FIG. 27D) 150 mL min⁻¹.

FIG. 28 shows distribution of the costs.

FIG. 29 shows distribution of the CAPEX of the units per section.

FIG. 30 shows effect of LDPE price on the MSP of lubricants. The green region corresponds to the prices below the minimum price reported for lubricants in the last decade.

The blue region is the region with prices in the last decade. The red region corresponds to the region above the maximum price observed for lubricants in the last decade.

FIG. 31 shows effect of the scale on the MSP of the lubricants for MW-Al case.

FIG. 32 shows MSP of lubricants under different MW power generator costs.

FIG. 33A-C shows selling price of the lubricant as a function of the IRR expected by the investor for different LDPE prices for a plant that processes 32 k Mt/y. Upper limit in the error bar is obtained with a pessimistic scenario (Cost of MW reactor of $700/kWh, cost of electricity ¢16.6/kWh, IF=2.5). Lower bound of the error bars is with cost of MW reactor of $300/kWh, cost of electricity ¢3.6/kWh, IF=1.1. FIG. 33A shows an LDPE price of $0.44/kg, FIG. 33B shows an LDPE price of $0.22/kg, and FIG. 33C shows an LDPE price of $0.78/kg.

FIG. 34 shows breakdown of the positive contributors to the impacts for MW-Al.

FIG. 35 shows breakdown of the positive contributors to the impacts for MW-Pt.

FIG. 36 shows breakdown of the positive contributors to the impacts for Monolith-Pt.

FIG. 37 shows breakdown of the positive contributors to the impacts for Monolith-Al.

FIG. 38 shows distribution of the impacts between the different process contributors for Conv.

FIG. 39 shows product yields from 76LDPE hydrocracking with 2 wt % select additives using a Ni/HBEA catalyst. Conditions: 1 h at 250° C., 60 bar H₂, a polymer-to-catalyst ratio of 50, and 2 wt % additive in the polymer.

FIG. 40 shows extended time reactivity for LDPE containing additives HALS and BT. Reaction conditions: 250° C., 60 bar H₂, 1 g 76LDPE, 20 mg additive, 20 mg Ni/HBEA, 24 h. No additive was added to the benchmark experiment.

FIG. 41 shows product selectivity and conversion of 76LDPE by hydrocracking with 2 wt % select additives using a Ni/HZSM-5 catalyst. Conditions: 1 h at 250° C., 60 bar H₂, a polymer-to-catalyst ratio of 50, and 2 wt % additive in the polymer.

FIG. 42 shows product selectivity and conversion from 76LDPE hydrogenolysis with 2 wt % additives using Ni/SiO₂. Reaction conditions: 250° C., 60 bar H₂, 1 g 76LDPE, 20 mg additive, 20 mg Ni/SiO₂, 1 h. No additive was used in the benchmark experiment.

FIG. 43A shows reactivity data of LDPE with various additives under melt catalytic pyrolysis conditions over HY. Reaction conditions: 370° C., 100 ml/min of N₂, polymer/catalyst=10, 2 wt % additive.

FIG. 43B shows reactivity data of LDPE with various additives under melt catalytic pyrolysis conditions over HZSM-5. Reaction conditions: 370°° C., 100 ml/min of N₂, polymer/catalyst=10, 2 wt % additive.

FIG. 43C shows reactivity data of LDPE with various additives under melt catalytic pyrolysis conditions over Al-SBA-15. Reaction conditions: 370° C., 100 ml/min of N₂, polymer/catalyst=10, 2 wt % additive.

FIG. 44A shows product distribution from catalytic pyrolysis of additive-containing LDPE over HY (reaction conditions: 370° C., 100 ml/min of N₂, polymer/catalyst=10, 2 wt % additive).

FIG. 44B shows product distribution from catalytic pyrolysis of additive-containing LDPE over HZSM-5 (reaction conditions: 370° C., 100 ml/min of N₂, polymer/catalyst=10, 2 wt % additive).

FIG. 44C shows shows product distribution from catalytic pyrolysis of additive-containing LDPE over Al-SBA-15 (reaction conditions: 370° C., 100 ml/min of N₂, polymer/catalyst=10, 2 wt % additive).

FIG. 45 shows reactivity data of catalyst HY after three times reuse. Reaction conditions: 370° C., 100 mL/min N₂, catalyst/polymer=10, 2 wt % of SA.

### DETAILED DESCRIPTION

### 1. Processes of Present Disclosure

a. Process for converting a plastic comprising a polyolefin polymer to an alkene

In a first embodiment, the present disclosure provides a pyrolysis process for converting a plastic comprising a polyolefin polymer to an alkene, comprising contacting the plastic with a catalyst in a one-pot pyrolysis system at a temperature between about 350° C. and about 500° C.; wherein the catalyst comprises a solid acid; and wherein the one-pot pyrolysis system comprises a microwave-assisted slurry reactor.

In a second embodiment, the present disclosure provides a process according to the first embodiment, wherein the plastic comprises a homopolymer of an olefin, a copolymer of olefins, or a mixture thereof. The definitions of the remaining variables are provided in the first embodiment and the other embodiments described herein.

In a third embodiment, the present disclosure provides a process according to the first embodiment or second embodiment, wherein the plastic comprises polyethylene, polypropylene, polybutene, polyisobutylene, polypentene, polyhexene, polyoctene, polystyrene, or a mixture thereof. The definitions of the remaining variables are provided in the first embodiment or the second embodiment and the other embodiments described herein.

In a fourth embodiment, the present disclosure provides a process according to any one of the first through third embodiments, wherein the plastic comprises high-density polyethylene (HDPE), low-density polyethylene (LDPE), polypropylene (PP), polystyrene (PS), or a mixture thereof. The definitions of the remaining variables are provided in any one of the first through third embodiment and the other embodiments described herein.

In a fifth embodiment, the present disclosure provides a process according to any one of the first through fourth embodiments, wherein the plastic is selected from the group consisting of isotactic polypropylene, syndiotactic polypropylene, atactic polypropylene, low molecular weight isotactic polypropylene, amorphous polypropylene, polypropylene bottles, polypropylene transparent bags, and a mixture thereof. The definitions of the remaining variables are provided in any one of the first through fourth embodiments and the other embodiments described herein.

In a sixth embodiment, the present disclosure provides a process according to any one of the first through fifth embodiments, wherein the plastic is selected from the group consisting of isotactic polypropylene, low molecular weight isotactic polypropylene, amorphous polypropylene, polypropylene bottles, polypropylene transparent bags, and a mixture thereof. The definitions of the remaining variables are provided in any one of the first through fifth embodiments and the other embodiments described herein.

In a seventh embodiment, the present disclosure provides a process according to any one of the first through sixth embodiments, wherein the pyrolysis process is conducted in a gas flow at a rate between about 5 ml/min and about 150 ml/min. The definitions of the remaining variables are provided in any one of the first through sixth embodiments and the other embodiments described herein. In one embodiment, the gas flow is at a rate between about 10 ml/min and about 150 ml/min, about 20 ml/min and about 150 ml/min, about 30 ml/min and about 150 ml/min, about 40 ml/min and about 150 ml/min, about 50 ml/min and about 150 ml/min, about 60 ml/min and about 150 ml/min, about 70 ml/min and about 150 ml/min, about 80 ml/min and about 150 ml/min, about 90 ml/min and about 150 ml/min, about 90 ml/min and about 140 ml/min, about 90 ml/min and about 130 ml/min, about 90 ml/min and about 120 ml/min, or about 90 ml/min and about 110 ml/min. In one embodiment, the gas flow is at a rate about 10 ml/min, about 15 ml/min, about 20 ml/min, about 25 ml/min, about 30 ml/min, about 35 ml/min, about 40 ml/min, about 45 ml/min, about 50 ml/min, about 55 ml/min, about 60 ml/min, about 65 ml/min, about 70 ml/min, about 75 ml/min, about 80 ml/min, about 85 ml/min, about 90 ml/min, about 95 ml/min, about 100 ml/min, about 105 ml/min, about 110 ml/min, about 115 ml/min, about 120 ml/min, about 125 ml/min, about 130 ml/min, about 135 ml/min, about 140 ml/min, about 145 ml/min, or about 150 ml/min.

In an eighth embodiment, the present disclosure provides a process according to the seventh embodiment, wherein the gas flow is at a rate between about 15 ml/min and about 125 ml/min. The definitions of the remaining variables are provided in the seventh embodiment and the other embodiments described herein.

In a ninth embodiment, the present disclosure provides a process according to the seventh and eighth embodiment, wherein the gas flow is at a rate between about 25 ml/min and about 100 ml/min. The definitions of the remaining variables are provided in the seventh and eighth embodiment or the other embodiments described herein.

In a tenth embodiment, the present disclosure provides a process according to any one of the seventh through ninth embodiments, wherein the gas flow is at a rate of about 25 ml/min, about 50 ml/min, about 75 ml/min, or about 100 ml/min. The definitions of the remaining variables are provided in any one of the seventh through ninth embodiments and the other embodiments described herein.

In an eleventh embodiment, the present disclosure provides a process according to any one of the seventh through tenth embodiments, wherein the gas flow is at a rate of about 100 ml/min. The definitions of the remaining variables are provided in any one of the seventh through tenth embodiments and the other embodiments described herein.

In a twelfth embodiment, the present disclosure provides a process according to any one of the seventh through eleventh embodiments, wherein the gas flow comprises nitrogen or hydrogen. The definitions of the remaining variables are provided in any one of the seventh through eleventh embodiments and the other embodiments described herein.

In a thirteenth embodiment, the present disclosure provides a process according to the twelfth embodiment, wherein the gas flow is a nitrogen gas flow. The definitions of the remaining variables are provided in the twelfth embodiments and the other embodiments described herein.

In a fourteenth embodiment, the present disclosure provides a process according to any one of the first through thirteenth embodiments, wherein the temperature is between about 350° C. and about 475° C. The definitions of the remaining variables are provided in any one of the first through thirteenth embodiments and the other embodiments described herein. In one embodiment, the temperature is between about 350° C. and about 450° C., about 350°° C. and about 425° C., about 350° C. and about 400° C., about 350° C. and about 375° C., about 375° C. and about 450° C., about 400° C. and about 450° C., or about 425° C. and about 450° C. In one embodiment, the temperature is about 350° C., about 355° C., about 360° C., about 365° C., about 370° C., about 375° C., about 380° C., about 385° C., about 390° C., about 395° C., about 400° C., about 405°° C., about 410°° C., about 415° C., about 420° C., about 425° C., about 430° C., about 435° C., about 440° C., about 445° C., about 450° C., about 455° C., about 460° C., about 465° C., about 470° C., about 475° C., about 480° C., about 485° C., about 495°° C., or about 500° C.

In a fifteenth embodiment, the present disclosure provides a process according to any one of the first through fourteenth embodiments, wherein the temperature is between about 350° C. and about 450° C. The definitions of the remaining variables are provided in any one of the first through fourteenth embodiments and the other embodiments described herein.

In a sixteenth embodiment, the present disclosure provides a process according to any one of the first through fifteenth embodiments, wherein the temperature is between about 350° C. and about 400° C. The definitions of the remaining variables are provided in any one of the first through fifteenth embodiments and the other embodiments described herein.

In a seventeenth embodiment, the present disclosure provides a process according to any one of the first through sixteenth embodiments, wherein the temperature is about 375° C.

In an eighteenth embodiment, the present disclosure provides a process according to any one of the first through seventeenth embodiments, wherein the pyrolysis process is conducted for a period of time of less than 10 minutes. In one embodiment, the period of time is between about 5 seconds and about 600 seconds, about 10 seconds and about 600 seconds, about 15 seconds and about 600 seconds, about 20 seconds and about 600 seconds, about 25 seconds and about 600 seconds, about 30 seconds and about 600 seconds, about 35 seconds and about 600 seconds, about 40 seconds and about 600 seconds, about 45 seconds and about 600 seconds, about 50 seconds and about 600 seconds, about 55 seconds and about 600seconds, about 60 seconds and about 600 seconds, about 65 seconds and about 600 seconds, about 70 seconds and about 600 seconds, about 75 seconds and about 600 seconds, about 80 seconds and about 600 seconds, about 85 seconds and about 600 seconds, about 90 seconds and about 600 seconds, about 95 seconds and about 600 seconds, about 100 seconds and about 600 seconds, about 105 seconds and about 600 seconds, about 110 seconds and about 600 seconds, about 115 seconds and about 600 seconds, about 120 seconds and about 600 seconds, about 125 seconds and about 600 seconds, about 130 seconds and about 600 seconds, about 135 seconds and about 600 seconds, about 140 seconds and about 600 seconds, about 145 seconds and about 600 seconds, about 150 seconds and about 600 seconds, about 155 seconds and about 600 seconds, about 160 seconds and about 600 seconds, about 165 seconds and about 600 seconds, about 170 seconds and about 600 seconds, about 175 seconds and about 600 seconds, about 180 seconds and about 600 seconds, about 185 seconds and about 600 seconds, about 190 seconds and about 600 seconds, about 195 seconds and about 600 seconds, about 200 seconds and about 600 seconds, about 200 seconds and about 550 seconds, about 200 seconds and about 500 seconds, about 200 seconds and about 450 seconds, about 200 seconds and about 400 seconds, about 200 seconds and about 350 seconds, about 200 seconds and about 300 seconds, about 200 seconds and about 250 seconds, or about 200 seconds and about 225 seconds. In one embodiment, the period of time is about 5 seconds, about 10 seconds, about 50 seconds, about 100 seconds, about 150 seconds, about 200 seconds, about 250 seconds, about 300 seconds, about 350 seconds, about 400 seconds, about 450 seconds, about 500 seconds, about 550 seconds, or about 600 seconds.

In a nineteenth embodiment, the present disclosure provides a process according to the eighteenth embodiment, wherein the period of time is between about 10 seconds and about 400 seconds. The definitions of the remaining variables are provided in the eighteenth embodiment and the other embodiments described herein.

In a twentieth embodiment, the present disclosure provides a process according to the eighteenth or nineteenth embodiment, wherein the period of time is between about 50 seconds and about 250 seconds. The definitions of the remaining variables are provided in the eighteenth or nineteenth embodiment and the other embodiments described herein.

In a twenty-first embodiment, the present disclosure provides a process according to any one of the eighteenth through twentieth embodiments, wherein the period of time is about 200 seconds. The definitions of the remaining variables are provided in the eighteenth through twentieth embodiments and the other embodiments described herein.

In a twenty-second embodiment, the present disclosure provides a process according to any one of the first through twenty-first embodiments, wherein the alkene is a mixture of C₄-C₁₂ alkenes. The definitions of the remaining variables are provided in any one of the first through twenty-first embodiments and the other embodiments described herein.

In a twenty-third embodiment, the present disclosure provides a process according to any one of the first through twenty-second embodiments, wherein the alkene is a mixture selected from the group consisting of C₄ alkenes, C₅ alkenes, C₇-C₁₂ alkenes, and a mixture thereof. The definitions of the remaining variables are provided in the first through twenty-second embodiments and the other embodiments described herein.

In a twenty-fourth embodiment, the present disclosure provides a process according to any one of the first through twenty-third embodiments, wherein the weight ratio between the plastic and the catalyst is about 40:1 to about 2:1. The definitions of the remaining variables are provided in any one of the first through twenty-third embodiments and the other embodiments described herein. In one embodiment, the weight ratio between the plastic and the catalyst is about 40:1 to about 2:1, about 35:1 to about 2:1, about 30:1 to about 25:1, about 20:1 to about 2:1, about 15:1 to about 2:1, about 10:1 to about 2:1, about 5:1 to about 2:1, about 3:1 to about 2:1, about 40:1 to about 5:1, about 40:1 to about 10:1, about 40:1 to about 15:1, about 40:1 to about 20:1, about 40:1 to about 25:1, about 40:1 to about 30:1, or about 40:1 to about 35:1. In one embodiment, the weight ratio between the plastic and the catalyst is about 40:1, about 35:1, about 30:1, about 25:1, about 20:1, about 15:1, about 10:1, about 5:1, about 3:1, or about 3:1.

In a twenty-fifth embodiment, the present disclosure provides a process according to any one of the first through twenty-fourth embodiments, wherein the weight ratio between the plastic and the catalyst is about 30:1 to about 5:1. The definitions of the remaining variables are provided in any one of the first through twenty-fourth embodiments and the other embodiments described herein.

In a twenty-sixth embodiment, the present disclosure provides a process according to any one of the first through twenty-fifth embodiments, wherein the weight ratio between the plastic and the catalyst is about 20:1 to about 8:1. The definitions of the remaining variables are provided in any one of the first through twenty-fifth embodiments and the other embodiments described herein.

In a twenty-seventh embodiment, the present disclosure provides a process according to any one of the first through twenty-sixth embodiments, wherein the weight ratio between the plastic and the catalyst is about 10:1. The definitions of the remaining variables are provided in the first through twenty-sixth embodiments and the other embodiments described herein.

In a twenty-eighth embodiment, the present disclosure provides a process according to any one of the first through twenty-seventh embodiments, wherein the conversion of the plastic is at least about 25%. The definitions of the remaining variables are provided in any one of the first through twenty-seventh embodiments and the other embodiments described herein. In one embodiment, the conversion of the plastic is at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

In a twenty-ninth embodiment, the present disclosure provides a process according to any one of the first through twenty-eighth embodiments, wherein the conversion of the plastic is at least about 40%. The definitions of the remaining variables are provided in any one of the first through twenty-eighth embodiments and the other embodiments described herein.

In a thirtieth embodiment, the present disclosure provides a process according to any one of the first through twenty-ninth embodiments, wherein the conversion of the plastic is at least about 60%. The definitions of the remaining variables are provided in any one of the first through twenty-ninth embodiments and the other embodiments described herein.

In a thirty-first embodiment, the present disclosure provides a process according to any one of the first through thirtieth embodiments, wherein the conversion of the plastic is at least about 90%. The definitions of the remaining variables are provided in any one of the first through thirtieth embodiments and other embodiments described herein.

In a thirty-second embodiment, the present disclosure provides a process according to any one of the first through thirty-first embodiment, wherein the yield of the alkene is at least about 25%. The definitions of the remaining variables are provided in any one of the first through thirty-first embodiments and other embodiments described herein. In one embodiment, the yield of the alkene is at least 30%, at least 35%, at least 40%, at least 45%, at least 50%, at least 55%, at least 60%, at least 65%, at least 70%, at least 75%, at least 80%, at least 85%, at least 90%, or at least 95%.

In a thirty-third embodiment, the present disclosure provides a process according to any one of the first through thirty-second embodiments, wherein the yield of the alkene is at least about 70%. The definitions of the remaining variables are provided in any one of the first through thirty-second embodiments and other embodiments described herein.

In a thirty-fourth embodiment, the present disclosure provides a process according to any one of the first through thirty-third embodiments, wherein the yield of the alkene is at least about 80%. The definitions of the remaining variables are provided in any one of the first through thirty-third embodiments and the other embodiments described herein.

In a thirty-fifth embodiment, the present disclosure provides a process according to any one of the first through thirty-fourth embodiments, wherein the catalyst is selected from the group consisting of P-SiO2, 15WZr, 25WZr, H-ZSM-5, Al-MCM-41, Al-SBA-15, and HY(30). The definitions of the remaining variables are provided in any one of the first through thirty-fourth embodiments and the other embodiments described herein.

In a thirty-sixth embodiment, the present disclosure provides a process according to any one of the first through thirty-fifth embodiments, wherein the process further comprises separating resulting liquid pyrolysis products from resulting gaseous pyrolysis products through a condenser section. The definitions of the remaining variables are provided in any one of the first through thirty-fifth embodiments and the other embodiments described herein.

In a thirty-seventh embodiment, the present disclosure provides a process according to any one of the first through thirty-sixth embodiments, wherein the microwave-assisted slurry reactor comprises a reaction vessel and a microwave source. The definitions of the remaining variables are provided in any one of the first through thirty-sixth embodiments and the other embodiments described herein.

In a thirty-eighth embodiment, the present disclosure provides a process according to the thirty-seventh embodiment, wherein the reaction vessel comprises
a plastic inlet for adding plastic; and
a gas inlet for injecting a gas flow through the reaction vessel.

The definitions of the remaining variables are provided in the thirty-seventh embodiment and the other embodiments described herein.

In a thirty-ninth embodiment, the present disclosure provides a process according to the thirty-seventh or thirty-eighth embodiments, wherein the reaction vessel is a tubular reactor.

In a fortieth embodiment, the present disclosure provides a process according to any one of the thirty-seventh through thirty-ninth embodiments, wherein the reaction vessel further comprises a porous frit. The definitions of the remaining variables are provided in any one of the thirty-seventh through thirty-ninth embodiments and the other embodiments described herein.

In a forty-first embodiment, the present disclosure provides a process according to the fortieth embodiments, wherein the porous frit is a porous quartz frit fixed in the reaction vessel. The definitions of the remaining variables are provided in the fortieth embodiments and the other embodiments described herein.

In a forty-second embodiment, the present disclosure provides a process according to any one of the thirty-seventh through forty-first embodiments, wherein the reaction vessel comprises a microwave susceptor. The definitions of the remaining variables are provided in any one of the thirty-seventh through forty-first embodiments and the other embodiments described herein.

In a forty-third embodiment, the present disclosure provides a process according to the forty-second embodiment, wherein the microwave susceptor is a SiC monolith. The definitions of the remaining variables are provided in the forty-second embodiment and the other embodiments described herein.

In a forty-fourth embodiment, the present disclosure provides a process according to any one of the thirty-seventh through forty-third embodiments, wherein the microwave source emitting microwaves to melt the plastic to form a slurry in the reaction vessel. The definitions of the remaining variables are provided in any one of the thirty-seventh through forty-third embodiments and the other embodiments described herein.

In a forty-fifth embodiment, the present disclosure provides a process according to any one of the thirty-seventh through forty-fourth embodiments, further comprising a temperature probe for measuring a core temperature within said reactor vessel. The definitions of the remaining variables are provided in any one of the thirty-seventh through forty-third embodiments and the other embodiments described herein.
b. Process for converting an additive-containing plastic comprising a polyolefin polymer to an alkene or an alkane

In some embodiments, the present disclosure provides a pyrolysis process for converting an additive-containing plastic comprising a polyolefin polymer to an alkene, an alkane, or a mixture thereof, comprising contacting the additive-containing plastic with a catalyst in a one-pot pyrolysis system at a temperature between about 350° C. and about 500° C.; wherein the catalyst comprises a solid acid selected from the group consisting of HY, HZSM-5, and Al-SBA-15; and wherein the one-pot pyrolysis system comprises a microwave-assisted slurry reactor.

In some embodiments, the additive-containing plastic of the present disclosure comprises a homopolymer of an olefin, a copolymer of olefins, or a mixture thereof. The definitions of the remaining variables are provided the other embodiments described herein.

In some embodiments, the additive-containing plastic of the present disclosure comprises polyethylene, polypropylene, polybutene, polyisobutylene, polypentene, polyhexene, polyoctene, polystyrene, or a mixture thereof. The definitions of the remaining variables are provided in the other embodiments described herein.

In some embodiments, the additive-containing plastic of the present disclosure comprises high-density polyethylene (HDPE), low-density polyethylene (LDPE), polypropylene (PP), polystyrene (PS), or a mixture thereof. The definitions of the remaining variables are provided in the other embodiments described herein.

In some embodiments, the additive-containing plastic of the present disclosure comprises isotactic polypropylene, syndiotactic polypropylene, atactic polypropylene, low molecular weight isotactic polypropylene, amorphous polypropylene, polypropylene bottles, polypropylene transparent bags, or a mixture thereof. The definitions of the remaining variables are provided in the other embodiments described herein.

In some embodiments, the additive-containing plastic of the present disclosure comprises isotactic polypropylene, low molecular weight isotactic polypropylene, amorphous polypropylene, polypropylene bottles, polypropylene transparent bags, and a mixture thereof. The definitions of the remaining variables are provided in the other embodiments described herein.

In some embodiments, the additive-containing plastic of the present disclosure comprises one or more additives, wherein the additive is an antioxidant, a light stabilizer, a ultraviolet (UV) light absorber, a filler, a slip agent, a lubricant additive, a stabilizer, or a mixture thereof. The definitions of the remaining variables are provided in the other embodiments described herein.

In some embodiments, the antioxidant of the present disclosure is butylated hydroxytoluene (BHT), 1,3,5-Tris(3,5-di-tert-butyl-4-hydroxybenzyl) (I-3114), Diphenyl Phosphite (SAO), tris(2,4-di-tert-butylphenyl) phosphite (SAO-C), or a mixture thereof. The definitions of the remaining variables are provided in the other embodiments described herein.

In some embodiments, the antioxidant of the present disclosure is Diphenyl Phosphite (SAO), tris(2,4-di-tert-butylphenyl) phosphite (SAO-C), or a mixture thereof. The definitions of the remaining variables are provided in the other embodiments described herein.

In some embodiments, the light stabilizer of the present disclosure is 2,2,6,6-Tetramethylpiperidine (HALS), bis(2,2,6,6-tetramethyl-4-piperidyl) sebacate (HALS-C), or a mixture thereof. The definitions of the remaining variables are provided in the other embodiments described herein.

In some embodiments, the UV light absorber of the present disclosure is 2-(2-Hydroxy-5-methylphenyl) benzotriazole (BT), 2-(2H-Benzotriazol-2-yl)-6-dodecyl-4-methylphenol (BT-C), or a mixture thereof. The definitions of the remaining variables are provided in the other embodiments described herein.

In some embodiments, the lubricant additive of the present disclosure is octadecanoic acid (L-C18A), 1-Octadecanol (L-C18OH), or a mixture thereof. The definitions of the remaining variables are provided in the other embodiments described herein.

In some embodiments, the slip agent of the present disclosure is oleamide (SA). The definitions of the remaining variables are provided in the other embodiments described herein.

In some embodiments, the additive is selected from the group consisting of compounds listed in Table A:

TABLE A

| No. | IUPAC Name | Abbreviation | Structure |
|---|---|---|---|
| 1 | Butylated Hydroxytoluene | BHT | 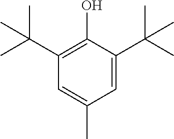 [SMILES: Cc1cc(C(C)(C)C)c(O)c(C(C)(C)C)c1] |
| 2 | 1,3,5-Tris(3,5-di-tert- butyl-4-hydroxybenzyl) | I-3114 | 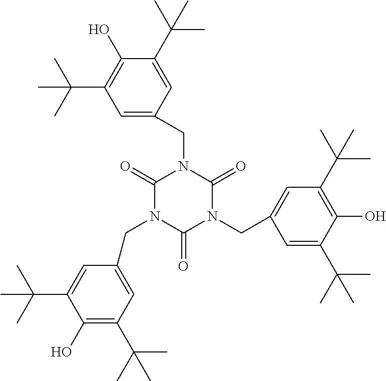 [SMILES: CC(C)(C)c1cc(Cn2c(=O)n(Cc3cc(C(C)(C)C)c(O)c(C(C)(C)C)c3)c(=O)n(Cc3cc(C(C)(C)C)c(O)c(C(C)(C)C)c3)c2=O)cc(C(C)(C)C)c1O] |
| 3 | Diphenyl Phosphite | SAO | 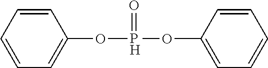 |
| 4 | Tris(2,4-di-tert- butylphenyl) phosphite | SAO-C | 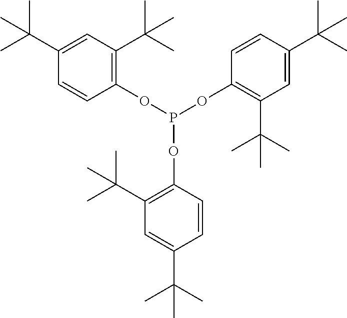 [SMILES: CC(C)(C)c1ccc(OP(Oc2ccc(C(C)(C)C)cc2C(C)(C)C)Oc2ccc(C(C)(C)C)cc2C(C)(C)C)c(C(C)(C)C)c1] |
| 5 | 2,2,6,6- Tetramethylpiperidine | HALS | 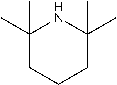 |
| 6 | Bis(2,2,6,6- tetramethyl-4- piperidyl) sebacate | HALS-C | 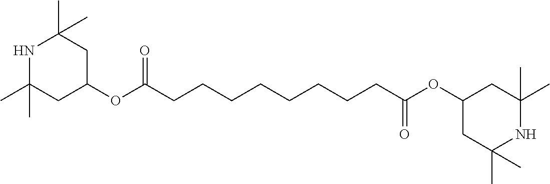 |
| 7 | 2-(2-Hydroxy-5- methylphenyl) benzotriazole | BT | 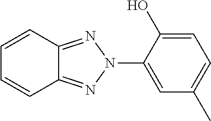 [SMILES: Cc1ccc(O)c(-n2nc3ccccc3n2)c1] |
| 8 | 2-(2H-Benzotriazol-2- yl)-6-dodecyl-4- methylphenol | BT-C | 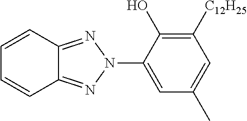 |
| 9 | Octadecanoic acid | L-C18A | 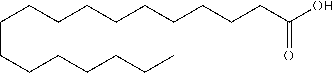 [SMILES: CCCCCCCCCCCCCCCCCC(=O)O] |
| 10 | 1-Octadecanol | L-C18OH | 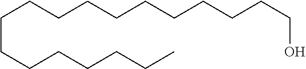 [SMILES: CCCCCCCCCCCCCCCCCCO] |
| 11 | Oleamide | SA | 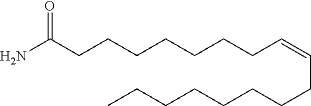 [SMILES: CCCCCCCC/C=C\CCCCCCCC(N)=O] |

In some embodiments, the catalyst of the present disclosure is HY. The definitions of the remaining variables are provided in the other embodiments described herein. In some embodiments, the HY catalyst comprises a micropore and a mesopore. In some embodiments, the pore size of the micropore in the HY catalyst is about 0.74 nm. In some embodiments, the pore size of the mesopore in the HY catalyst is about 5.1 nm.

In some embodiments, the catalyst of the present disclosure is HZSM-5. The definitions of the remaining variables are provided in the other embodiments described herein. In some embodiments, the HZSM-5 catalyst comprises a micropore. In some embodiments, the pore size of the micropore in the HZSM-5 catalyst is about 0.55 nm.

In some embodiments, the catalyst of the present disclosure is Al-SBA-15. The definitions of the remaining variables are provided in the other embodiments described herein.

In some embodiments, the pyrolysis process of the present disclosure is conducted in a gas flow at a rate between about 5 ml/min and about 150 ml/min. The definitions of the remaining variables are provided in the other embodiments described herein. In one embodiment, the gas flow is at a rate between about 10 ml/min and about 150 ml/min, about 20 ml/min and about 150 ml/min, about 30 ml/min and about 150 ml/min, about 40 ml/min and about 150 ml/min, about 50 ml/min and about 150 ml/min, about 60 ml/min and about 150 ml/min, about 70 ml/min and about 150 ml/min, about 80 ml/min and about 150 ml/min, about 90 ml/min and about 150 ml/min, about 90 ml/min and about 140 ml/min, about 90 ml/min and about 130 ml/min, about 90 ml/min and about 120 ml/min, or about 90 ml/min and about 110 ml/min. In one embodiment, the gas flow is at a rate about 10 ml/min, about 15 ml/min, about 20 ml/min, about 25 ml/min, about 30 ml/min, about 35 ml/min, about 40 ml/min, about 45 ml/min, about 50 ml/min, about 55 ml/min, about 60 ml/min, about 65 ml/min, about 70 ml/min, about 75 ml/min, about 80 ml/min, about 85 ml/min, about 90 ml/min, about 95 ml/min, about 100 ml/min, about 105 ml/min, about 110 ml/min, about 115 ml/min, about 120 ml/min, about 125 ml/min, about 130 ml/min, about 135 ml/min, about 140 ml/min, about 145 ml/min, or about 150 ml/min.

In some embodiments, the gas flow is at a rate between about 15 ml/min and about 125 ml/min. The definitions of the remaining variables are provided in the other embodiments described herein.

In some embodiments, the gas flow is at a rate between about 25 ml/min and about 100 ml/min. The definitions of the remaining variables are provided in the other embodiments described herein.

In some embodiments, the gas flow is at a rate of about 25 ml/min, about 50 ml/min, about 75 ml/min, or about 100 ml/min. The definitions of the remaining variables are provided in the other embodiments described herein.

In some embodiments, the gas flow is at a rate of about 100 ml/min. The definitions of the remaining variables are provided in the other embodiments described herein.

In some embodiments, the gas flow comprises nitrogen or hydrogen. The definitions of the remaining variables are provided in the other embodiments described herein.

In some embodiments, the gas flow is a nitrogen gas flow. The definitions of the remaining variables are provided in the other embodiments described herein.

In some embodiments, the pyrolysis process of the present disclosure is conducted in a temperature between about 350° C. and about 475° C. The definitions of the remaining variables are provided in the other embodiments described herein. In one embodiment, the temperature is between about 350° C. and about 450° C., about 350° C. and about 425° C., about 350° C. and about 400° C., about 350° C. and about 375° C., about 375° C. and about 450° C., about 400° C. and about 450° C., or about 425° C. and about 450° C. In one embodiment, the temperature is about 350° C., about 355° C., about 360° C., about 365° C., about 370° C., about 375° C., about 380° C., about 385° C., about 390° C., about 395° C., about 400° C., about 405° C., about 410° C., about 415° C., about 420° C., about 425° C., about 430° C., about 435° C., about 440° C., about 445° C., about 450° C., about 455° C., about 460° C., about 465° C., about 470° C., about 475° C., about 480° C., about 485° C., about 495° C., or about 500° C.

In some embodiments, the temperature is between about 350° C. and about 450° C. The definitions of the remaining variables are provided in the other embodiments described herein.

In some embodiments, the temperature is between about 350° C. and about 400° C. The definitions of the remaining variables are provided in the other embodiments described herein.

In some embodiments, the temperature is about 375° C. The definitions of the remaining variables are provided in the other embodiments described herein.

In some embodiments, the temperature is about 370° C. The definitions of the remaining variables are provided in the other embodiments described herein.

In some embodiments, the pyrolysis process of the present disclosure is conducted for a period of time of less than 10 minutes. In one embodiment, the period of time is between about 5 seconds and about 600 seconds, about 10 seconds and about 600 seconds, about 15 seconds and about 600 seconds, about 20 seconds and about 600 seconds, about 25 seconds and about 600 seconds, about 30 seconds and about 600 seconds, about 35 seconds and about 600 seconds, about 40 seconds and about 600 seconds, about 45 seconds and about 600 seconds, about 50 seconds and about 600 seconds, about 55 seconds and about 600 seconds, about 60 seconds and about 600 seconds, about 65 seconds and about 600 seconds, about 70 seconds and about 600 seconds, about 75 seconds and about 600 seconds, about 80 seconds and about 600 seconds, about 85 seconds and about 600 seconds, about 90 seconds and about 600 seconds, about 95 seconds and about 600 seconds, about 100 seconds and about 600 seconds, about 105 seconds and about 600 seconds, about 110 seconds and about 600 seconds, about 115 seconds and about 600 seconds, about 120 seconds and about 600 seconds, about 125 seconds and about 600 seconds, about 130 seconds and about 600 seconds, about 135 seconds and about 600 seconds, about 140 seconds and about 600 seconds, about 145 seconds and about 600 seconds, about 150 seconds and about 600 seconds, about 155 seconds and about 600 seconds, about 160 seconds and about 600 seconds, about 165 seconds and about 600 seconds, about 170 seconds and about 600 seconds, about 175 seconds and about 600 seconds, about 180 seconds and about 600 seconds, about 185 seconds and about 600 seconds, about 190 seconds and about 600 seconds, about 195 seconds and about 600 seconds, about 200 seconds and about 600 seconds, about 200 seconds and about 550 seconds, about 200 seconds and about 500 seconds, about 200 seconds and about 450 seconds, about 200 seconds and about 400 seconds, about 200 seconds and about 350 seconds, about 200 seconds and about 300 seconds, about 200 seconds and about 250 seconds, or about 200 seconds and about 225 seconds. In one embodiment, the period of time is about 5 seconds, about 10 seconds, about 50 seconds, about 100 seconds, about 150 seconds, about 200 seconds, about 250 seconds, about 300 seconds, about 350 seconds, about 400 seconds, about 450 seconds, about 500 seconds, about 550 seconds, or about 600 seconds. The definitions of the remaining variables are provided in the other embodiments described herein.

In some embodiments, the period of time is between about 10 seconds and about 400 seconds. The definitions of the remaining variables are provided in the other embodiments described herein.

In some embodiments, the period of time is between about 50 seconds and about 250 seconds. The definitions of the remaining variables are provided in the other embodiments described herein.

In some embodiments, the period of time is about 200 seconds. The definitions of the remaining variables are provided in the other embodiments described herein.

In some embodiments, the alkene produced by the process of the present disclosure is a mixture of C₂-C₁₂ alkenes. The definitions of the remaining variables are provided in the other embodiments described herein.

In some embodiments, the alkene produced by the process of the present disclosure is a mixture selected from the group consisting of C₂ alkene, C₄ alkenes, C₅ alkenes, C₇-C₁₂ alkenes, and a mixture thereof. The definitions of the remaining variables are provided in the other embodiments described herein.

In some embodiments, the alkene produced by the process of the present disclosure is a mixture selected from the group consisting of C₄ alkenes, C₅ alkenes, C₇-C₁₂ alkenes, and a mixture thereof.

In some embodiments, the alkane produced by the process of the present disclosure is a mixture of C₁-C₁₂ alkanes. The definitions of the remaining variables are provided in the other embodiments described herein. In some embodiments, the mixture C₁-C₁₂ alkanes are the mixture of mono and di-branched alkanes.

In some embodiments, the alkane produced by the process of the present disclosure is a mixture of C₃-C₈ alkanes. The definitions of the remaining variables are provided in the other embodiments described herein.

In some embodiments, the weight ratio between the additive-containing plastic and the catalyst in the process of the present disclosure is about 40:1 to about 2:1. The definitions of the remaining variables are provided in the other embodiments described herein. In one embodiment, the weight ratio between the additive-containing plastic and the catalyst is about 40:1 to about 2:1, about 35:1 to about 2:1, about 30:1 to about 25:1, about 20:1 to about 2:1, about 15:1 to about 2:1, about 10:1 to about 2:1, about 5:1 to about 2:1, about 3:1 to about 2:1, about 40:1 to about 5:1, about 40:1 to about 10:1, about 40:1 to about 15:1, about 40:1 to about 20:1, about 40:1 to about 25:1, about 40:1 to about 30:1, or about 40:1 to about 35:1. In one embodiment, the weight ratio between the additive-containing plastic and the catalyst is about 40:1, about 35:1, about 30:1, about 25:1, about 20:1, about 15:1, about 10:1, about 5:1, about 3:1, or about 3:1.

In some embodiments, the weight ratio between the additive-containing plastic and the catalyst is about 30:1 to about 5:1. The definitions of the remaining variables are provided in the other embodiments described herein.

In some embodiments, the weight ratio between the additive-containing plastic and the catalyst is about 20:1 to about 8:1. The definitions of the remaining variables are provided in the other embodiments described herein.

In some embodiments, the weight ratio between the additive-containing plastic and the catalyst is about 10:1. The definitions of the remaining variables are provided in the other embodiments described herein.

In some embodiments, the conversion of the additive-containing plastic in the process of the present disclosure is at least about 25%. The definitions of the remaining variables are provided in any one of the first through twenty-seventh embodiments and the other embodiments described herein. In one embodiment, the conversion of the additive-containing plastic in the process of the present disclosure is at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

In some embodiments, the conversion of the additive-containing plastic in the process of the present disclosure is at least about 40%. The definitions of the remaining variables are provided in the other embodiments described herein.

In some embodiments, the conversion of the additive-containing plastic in the process of the present disclosure is at least about 60%. The definitions of the remaining variables are provided in the other embodiments described herein.

In some embodiments, the conversion of the additive-containing plastic in the process of the present disclosure is at least about 90%. The definitions of the remaining variables are provided in the other embodiments described herein.

In some embodiments, the yield of the alkene in the process of the present disclosure is at least about 25%. The definitions of the remaining variables are provided in the other embodiments described herein. In one embodiment, the yield of the alkene is at least 30%, at least 35%, at least 40%, at least 45%, at least 50%, at least 55%, at least 60%, at least 65%, at least 70%, at least 75%, at least 80%, at least 85%, at least 90%, or at least 95%.

In some embodiments, the yield of the alkene in the process of the present disclosure is at least about 70%. The definitions of the remaining variables are provided in the other embodiments described herein.

In some embodiments, the yield of the alkene in the process of the present disclosure is at least about 80%. The definitions of the remaining variables are provided in the other embodiments described herein.

In some embodiments, the yield of the alkane in the process of the present disclosure is about 10%. The definitions of the remaining variables are provided in the other embodiments described herein. In one embodiment, the yield of the alkane is at least 15%, at least 20%, at least 25%, at least 30%, at least 35%, at least 40%, at least 45%, at least 50%, at least 55%, at least 60%, at least 65%, at least 70%, or at least 75%.

In some embodiments, the process described herein further comprises separating resulting liquid pyrolysis products from resulting gaseous pyrolysis products through a condenser section. The definitions of the remaining variables are provided in the other embodiments described herein.

In some embodiments, the microwave-assisted slurry reactor described in the process of the present disclosure comprises a reaction vessel and a microwave source. The definitions of the remaining variables are provided in the other embodiments described herein.

### 2. One-pot Pyrolysis System

In a forty-sixth embodiment, the present disclosure provides a one-pot pyrolysis system comprising
a microwave-assisted slurry reactor; and
a condenser section;
wherein the microwave-assisted slurry reactor comprises a reaction vessel and a microwave source.

In a forty-seventh embodiment, the present disclosure provides a one-pot pyrolysis system according to the forty-sixth embodiment, wherein the reaction vessel comprises
a plastic inlet for adding plastic; and
a gas inlet for injecting a gas flow through the reaction vessel.

The definitions of the remaining variables are provided in the forty-sixth embodiment and the other embodiments described herein.

In a forty-eighth embodiment, the present disclosure provides a one-pot pyrolysis system according to the forty-sixth or forty-seventh embodiment, wherein the reaction vessel is a tubular reactor. The definitions of the remaining variables are provided in the forty-sixth or forty-seventh embodiment and the other embodiments described herein.

In a forty-ninth embodiment, the present disclosure provides a one-pot pyrolysis system according to any one of the forty-sixth through forty-eighth embodiments, wherein the reaction vessel further comprises a porous frit. The definitions of the remaining variables are provided in any one of the forty-sixth through forty-eighth embodiments and the other embodiments described herein.

In a fiftieth embodiment, the present disclosure provides a one-pot pyrolysis system according to the forty-ninth embodiment, wherein the porous frit is a porous quartz frit fixed in the reaction vessel. The definitions of the remaining variables are provided in the forty-ninth embodiment and the other embodiments described herein.

In a fifty-first embodiment, the present disclosure provides a one-pot pyrolysis system according to any one of the forty-sixth through fiftieth embodiments, wherein the reaction vessel comprises a microwave susceptor. The definitions of the remaining variables are provided in any one of the forty-sixth through fiftieth embodiments and the other embodiments described herein.

In a fifty-second embodiment, the present disclosure provides a one-pot pyrolysis system according to the fifty-first embodiment, wherein the microwave susceptor is a SiC monolith. The definitions of the remaining variables are provided in the fifty-first embodiments and the other embodiments described herein.

In a fifty-third embodiment, the present disclosure provides a one-pot pyrolysis system according to any one of the forty-sixth through fifty-second embodiments, wherein the microwave source emitting microwaves to melt the plastic to form a slurry in the reaction vessel. The definitions of the remaining variables are provided in any one of the forty-sixth through fifty-second embodiments and the other embodiments described herein.

In a fifty-fourth embodiment, the present disclosure provides a one-pot pyrolysis system according to any one of the forty-sixth through fifty-third embodiments, further comprising a temperature probe for measuring a core temperature within said reactor vessel. The definitions of the remaining variables are provided in any one of the forty-sixth through fifty-third embodiments and the other embodiments described herein.

In a fifty-fifth embodiment, the present disclosure provides a one-pot pyrolysis system according to any one of the forty-sixth through fifty-fourth embodiments, wherein the condenser section is connected with the microwave-assisted slurry reactor through a connection means. The definitions of the remaining variables are provided in any one of the forty-sixth through fifty-fourth embodiments and the other embodiments described herein.

In a fifty-sixth embodiment, the present disclosure provides a one-pot pyrolysis system according to the fifty-fifth embodiment, wherein the connection means is a glass tube. The definitions of the remaining variables are provided in the fifty-fifth embodiment and the other embodiments described herein.

In a fifty-seventh embodiment, the present disclosure provides a one-pot pyrolysis system according to the fifty-sixth embodiment, wherein the glass tube is surrounded by heating bands. The definitions of the remaining variables are provided in the fifty-sixth embodiment and the other embodiments described herein.

In a fifty-eighth embodiment, the present disclosure provides a one-pot pyrolysis system according to any one of the forty-sixth through fifty-seventh embodiments, wherein the condenser section separates liquid pyrolysis products from gaseous pyrolysis products. The definitions of the remaining variables are provided in any one of the forty-sixth through fifty-seventh embodiments and the other embodiments described herein.

In a fifty-ninth embodiment, the present disclosure provides a one-pot pyrolysis system according to the fifty-eighth embodiment, wherein the condenser section comprises at least one cooling system. The definitions of the remaining variables are provided in the fifty-eighth embodiment and the other embodiments described herein.

In a sixtieth embodiment, the present disclosure provides a one-pot pyrolysis system according to the fifty-ninth embodiment, wherein the at least one cooling system is a water cooling system. The definitions of the remaining variables are provided in the fifty-ninth embodiment and the other embodiments described herein.

In a sixty-first embodiment, the present disclosure provides a one-pot pyrolysis system according to the fifty-eighth through sixtieth embodiments, wherein the condenser section comprises at least one condenser. The definitions of the remaining variables are provided in any one of the fifty-eighth through sixtieth embodiments and the other embodiments described herein.

In a sixty-second embodiment, the present disclosure provides a one-pot pyrolysis system according to any one of the forty-sixth through sixty-first embodiments, wherein the condenser section connects to a gas collection element and a liquid collection element. The definitions of the remaining variables are provided in any one of the forty-sixth through sixty-first embodiments and the other embodiments described herein.

In a sixty-third embodiment, the present disclosure provides a one-pot pyrolysis system according to the sixty-second embodiment, wherein the gas collection element is a gas bag. The definitions of the remaining variables are provided in the sixty-second embodiment and the other embodiments described herein.

In a sixty-fourth embodiment, the present disclosure provides a one-pot pyrolysis system according to the sixty-third embodiment, wherein the liquid collection element is a glass bottle. The definitions of the remaining variables are provided in the sixty-third embodiments and the other embodiments described herein.

In a sixty-fifth embodiment, the present disclosure provides a one-pot pyrolysis system according to the sixty-fourth embodiment, wherein the glass bottle is immersed in an ice bath. The definitions of the remaining variables are provided in the sixty-fourth embodiment and the other embodiments described herein.

### 3. Use of Solid Acid in Pyrolysis Process

a. Use of solid acid in pyrolysis process for converting plastic comprising polyolefin polymer to alkene

In a sixty-sixth embodiment, the present disclosure provides a use of a solid acid in a pyrolysis process for converting plastic comprising a polyolefin polymer to an alkene; wherein the solid acid is selected from the group consisting of P-SiO2, 15WZr, 25WZr, H-ZSM-5, Al-MCM-41, Al-SBA-15, and HY(30).

In a sixty-seventh embodiment, the present disclosure provides a use according to the sixty-sixth embodiment, wherein the pyrolysis process comprises contacting the plastic with a catalyst in a one-pot pyrolysis system at a temperature between about 350° C. and about 500° C.

In a sixty-eighth embodiment, the present disclosure provides a use according to the sixty-seventh embodiment, wherein the one-pot pyrolysis system comprises a microwave-assisted slurry reactor. The definitions of the remaining variables are provided in the sixty-seventh embodiments and the other embodiments described herein.
b. Use of solid acid in pyrolysis process for converting plastic comprising polyolefin polymer to alkene

In some embodiments, the present disclosure provides a use of a solid acid in a pyrolysis process for converting an additive-containing plastic comprising a polyolefin polymer to an alkene, an alkane, or a mixture thereof; wherein the solid acid is selected from the group consisting of HY, HZSM-5, and Al-SBA-15. The definitions of the remaining variables are provided in the other embodiments described herein.

In some embodiments, the pyrolysis process described herein comprises contacting the additive-containing plastic with a catalyst in a one-pot pyrolysis system at a temperature between about 350° C. and about 500° C. The definitions of the remaining variables are provided in the other embodiments described herein.

In some embodiments, the one-pot pyrolysis system comprises a microwave-assisted slurry reactor. The definitions of the remaining variables are provided in the other embodiments described herein.

### 4. Definitions

To facilitate understanding of the disclosure set forth herein, a number of terms are defined below. Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In this specification and the appended claims, the singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “alkene” or “olefin” as used herein generally refers to a monovalent group derived from a C₂₋₁₂ inclusive straight or branched hydrocarbon having at least one carbon-carbon double bond by the removal of a single hydrogen molecule. Examples of alkenes include, but are not limited to, ethene, propene, butene, pentene, hexene, heptane, octene, nonene, and decene higher homologs and isomers.

The term “alkane” as used herein generally refers to a saturated straight hydrocarbon, optionally branched, which contains the number of carbon atoms specified in the description or claims. Examples include, but does not limit to, n-butane, isobutane, pentane, hexane, methylbutane, heptane and octane.

The term “additive” as used herein generally refers to a material which is intended to be added to a polymer material before the forming thereof in order to modify one or more chemical/physical characteristics thereof. In some embodiments, the additive is an antioxidant, a light stabilizer, a ultraviolet (UV) light absorber, a filler, a slip agent, a lubricant additive, a stabilizer, or a mixture thereof. Examples include, but do not limit to, butylated hydroxytoluene (BHT), 1,3,5-Tris(3,5-di-tert-butyl-4-hydroxybenzyl) (I-3114), Diphenyl Phosphite (SAO), tris(2,4-di-tert-butylphenyl) phosphite (SAO-C), 2,2,6,6-Tetramethylpiperidine (HALS), bis(2,2,6,6-tetramethyl-4-piperidyl) sebacate (HALS-C), 2-(2-Hydroxy-5-methylphenyl) benzotriazole (BT), 2-(2H-Benzotriazol-2-yl)-6-dodecyl-4-methylphenol (BT-C), octadecanoic acid (L-C18A), 1-Octadecanol (L-C18OH), oleamide (SA), or a mixture thereof. The weight ratio of additive materials to a polymer material is generally less than 10%, less than 10%, or less than 5%. In some embodiments, the weight ratio of additive materials to a polymer material is about 2%.

The term “plastics” as used herein generally refers to a material based on organic macromolecules composed mainly of carbon and hydrogen, such as polyolefins, or also comprising oxygen, such as polyesters, polyethers, acrylic and methacrylic polymers, polyacetals, or macromolecules also comprising nitrogen, such as polyamides and polyurethanes, or macromolecules also comprising halogens, such as polyvinyl chloride and fluorinated polymers, or sulfur-containing macromolecules, such as polysulfides and polysulfones, or copolymers obtained by combining various monomers, such as acrylonitrile-butadiene copolymers (ABS) and like. In one embodiment, the plastics used in the present disclosure are recycled plastics, i.c. recovered from household and/or industrial waste by appropriate mechanical selection and grinding operations, as is known in the art. It therefore also can contain various additives and other components used in the production of the articles from which the recycled plastic derives. In one embodiment, the carbon content of the plastic used is greater than 45% by weight, greater than 60% by weight, or greater than 70% by weight. In one embodiment, the hydrogen content of the plastic used is greater than 5% by weight, greater than 8% by weight, or greater than 12% by weight. In one embodiment, the oxygen content is less than 20% by weight, less than 10% by weight, or less than 7% by weight. In one embodiment, the content of nitrogen, halogens and sulfur is overall less than 3% by weight, less than 2% by weight, or it is less than 0.5% by weight.

The term “antioxidant” as used herein generally refers to a chemical that combats oxidative degradation. Suitable antioxidants include butylated hydroxytoluene (BHT), 1,3,5-Tris(3,5-di-tert-butyl-4-hydroxybenzyl) (I-3114), Diphenyl Phosphite (SAO), tris(2,4-di-tert-butylphenyl)phosphite (SAO-C), or a mixture thereof.

The term “light stabilizer” as used herein generally refers to materials that have functionality of protecting plastics against degradation from long term exposure to light/UV radiation. Examples include, but do not limit to, 2.2.6,6-Tetramethylpiperidine (HALS), bis(2,2,6,6-tetramethyl-4-piperidyl) sebacate (HALS-C), or a mixture thereof.

The term “UV light absorber” as used herein generally refers to a chemical that absorbs UV light and minimizes the contact between plastic surface and the UV rays. In some embodiments, a UV light absorber is added to combat the deterioration of the plastic and significantly extends the life span of the final product. Examples include, but do not limit to, 2-(2-Hydroxy-5-methylphenyl) benzotriazole (BT), 2-(2H-Benzotriazol-2-yl)-6-dodecyl-4-methylphenol (BT-C), or a mixture thereof.

The term “lubricant additive” as used herein generally refers to a material that is used to improve the flow characteristics of plastics during processing. In some embodiments, lubricant additive is a compound that is chemically compatible with the polymer and acts by reducing friction between polymer molecules. It reduces Van der Waals forces, leading to a lower melt viscosity and lowering energy input needed for processing. Examples include, but do not limit to, octadecanoic acid (L-C18A), 1-Octadecanol (L-C18OH), or a mixture thereof.

The term “slip agent” as used herein generally refers to compounds that are added to polymers during manufacturing to control friction. In some embodiments, slip agents can reduce the coefficient of friction (CoF) and adhesion between the mold and injection molded polymer. They can also improve the surface smoothness of plastic films, which can improve their processing performance and extend their service life. Examples include, but do not limit to, oleamide.

The term “polyolefin polymer” as used herein generally refers to all polymers and copolymers (including high pressure low density polyethylene (LDPE), heterogeneous polymers, random, block, and graft polymers, interpolymers and copolymers) comprising one or more polymerized monomers selected from the group consisting of ethylene, an alpha olefin having from 3-20 carbon atoms (such as 1-propylene, 1-butene, 1-hexene, styrene, 1-heptene and 1-octene), 4-methyl-1-pentene, and/or acetylenically unsaturated monomers having from 2-20 carbons, and/or diolefins having from 4-18 carbons and any other monomer used in the art to modify the density of a polymer. Heterogeneous polymers include Ziegler-Natta polymerized polymers such as LLDPE and HDPE and include products such as DOWLEX™ Linear Low-Density Polyethylene (LLDPE) made by The Dow Chemical Company. The random copolymers include those polymerized using metallocene or constrained geometry catalyst technology and include polymers such as AFFINITY™ Polyolefin Plastomer and ENGAGE™ Polyolefin Elastomer both available from The Dow Chemical Company, and EXACT™ Polyolefin available from Exxon-Mobil. Methods for polymerizing these random copolymers are well known in the art and include those described in U.S. Pat. Nos. 5,272,236 and 5,278,272. The block copolymers include those polymerized using chain shuttling technology and two catalyst species, such as is disclosed in U.S. Pat. No. 7,355,089, and include polymers such as INFUSE™M Olefin Block Copolymers made by The Dow Chemical Company. In addition the term “polyolefin polymer” in this disclosure is defined as a polymer having an average molecular weight, as determined by light scattering, greater than 1,000 grams per mole (in one embodiment, 2,000 grams per mole, greater than 4,000 grams per mole, or can be as high as 10 million grams per mole). The polyolefin polymer can be a copolymer consisting essentially of polymerized ethylene monomer and a polymerized alpha olefin monomer such as 1-octene. The polyolefin polymer can be a copolymer consisting essentially of polymerized propylene monomer and a polymerized alpha olefin monomer such as ethylene. Such propylene-based polymers include homopolymer polypropylene, impact propylene-based copolymers, and random propylene based copolymers. Other more specialized polymers include ethylene/acrylic acid copolymers, ethylene/vinyl acetate copolymers and ethylene/styrene interpolymers, halogenated polymers, and polymers containing maleic anhydride mocities.

The term “polypropylene” means polyolefin containing more than 50.0% (by number) recurring propylene-derived units. In one embodiment, polypropylene homopolymer and/or polypropylene copolymer wherein at least 85% (by number) of the recurring units are propylene units. In one embodiment, polypropylene as used herein refers to a polymer consisting of 100% recurring propylene units.

The term “isotactic polypropylene” as used herein generally refers to a polypropylene where pendant groups (c.g., alkyl group such as methyl group) are oriented on one side of the carbon backbone, or at least 10%, 20%, 30%, 40%, 50%, 60%, 80%, 90%, or greater of all methyl groups oriented on one side of the carbon backbone, such that the isotactic polypropylene has greater structural rigidity or crystallinity to non-isotactic polymer (e.g., polypropylene).

The term “amorphous polypropylene” or “atactic polypropylene” as used herein generally refers to a polypropylene having random orientation of the pendant groups (e.g., alkyl groups such as methyl groups) along the polymer chain. What is meant by “amorphous” refers to be non-crystalline, for example, not having definite form nor apparent structural rigidity. The atactic polypropylene may be a random copolymer obtained, or obtainable, by polymerization of a homopolypropylene with one comonomer selected from a group consisting of propylene, ethylene, butylenes, and octene, or a block copolymer of polypropylene and ethylene-propylene.

The term “syndiotactic polypropylene” as used herein generally refers to polypropylene in which the substituents (e.g., alkyl group such as methyl group) have alternating positions along the polymer chain. In one embodiment, the term “syndiotactic polypropylene” is defined as having 10% or more syndiotactic pentads.

The term “microwave susceptor” as used herein generally refers to a material that redirects electromagnetic materials toward itself. A microwave susceptor may be made of a silicon carbide material, as an example, although any material having such properties may be used. In some examples, a microwave susceptor may include a material that absorbs microwave energy (e.g., a microwave sponge). The material of a microwave susceptor may reach temperatures of 200+° C. within 1 minute of microwaving, as an example, although other variations and material properties are possible.

The term “lubricant” as used herein generally refers to a substance that can be introduced between two or more moving surfaces and lowers the level of friction between two adjacent surfaces moving relative to each other. In one embodiment, it refers to a mixture of hydrocarbons having a carbon number distribution between about 13 and about 60.

The term “carbon number distribution” as used herein generally refers to the range of compounds present in a composition, wherein each compound is defined by the number of carbon atoms present.

### EXAMPLES

Useful embodiments of processes of the disclosure are provided in the following Examples. It should be understood that the Examples are given by way of illustration only.

### General Materials and Methods

a. Materials

Low-density polyethylene (MW 4000 Da), chloroplatinic acid (H₂PtCl₆, 8 wt % in H₂O), ethyl alcohol (200 proof), zirconyl chloride hydrate (ZrOCl₂·xH₂O, >99%), ammonium hydroxide (NH₄OH), phosphoric acid (H₃PO₄), ammonium metatungstate hydrate (99.99% trace metals basis), silica (mesoporous SBA-15, <150 μm particle size, pore size 6 nm), silica gel (high purity grade, pore size 60 Å, 70-230 mesh) and aluminum chloride (AlCl₃, >99%) were obtained from Sigma-Aldrich. n-octacosane was obtained from Tokyo Chemical Industry. Zeolite-Y (CBV720, Si:Al=30, H-form) was obtained from Zeolyst International. Al-MCM-41 (Si:Al=25) was obtained from ACS materials. Methylene chloride was obtained from Fisher Scientific. Ultrapure (type 1) water was used (Direct-Q 3 UV-R). All chemicals were used as received.

b. Catalyst preparation
b-1. Synthesis of 0.5 Pt-HY (30)

Zeolite HY(30) was calcined at 550° C. in air for 4 hr (2 C/min ramp) prior to use. Pt on HY(30) catalyst was synthesized by wetness impregnation of the HY(30) support with a chloroplatinic acid (H₂PtCl₆, 8 wt % in H₂O; Sigma-Aldrich) solution. 2.0 g of HY(30) was impregnated with a 0.064 M solution of chloroplatinic acid. The impregnated material then dried in air at 110° C., and then calcined at 550° C. for 4 hr (2° C./min ramp) in static air. The catalyst was loaded with 0.5 wt % Pt.
b-2. Synthesis of P-SiO₂

The P-SiO₂ catalyst (H₃PO₄, 10 wt %) was prepared by impregnation. First, SiO₂ (high purity grade, pore size 60 Å, 70-230 mesh, Sigma-Aldrich) was impregnated with an aqueous H₃PO₄ solution. After evaporating the solvent at 75° C. on a hotplate and subsequently drying at 110° C. for 12 hr in an oven, the fine powder catalyst was calcined in a crucible in air at 500° C. for 3 hr (2° C./min).
b-3. Synthesis of WO₃/ZrO₂

Firstly, zirconium (IV) hydroxide (Zr(OH)₄) was prepared via precipitation of ZrOCl₂ with NH₄OH. The precipitates were then aged for 24 hr in ultra-pure deionized (UPDI) water, adjusted to pH 10 by addition of NH₄OH, filtered, and then dried at 110° C. overnight. The thus obtained solids were crushed and subjected to consecutive redispersions in UPDI water (adjusted to pH 10 for 30 min) and filtrations to remove the Cl⁻ ions until the supernatant had background levels of Cl⁻ ions. The Cl⁻ ion concentration was tested using a 0.1 M AgNO₃ solution.

The final filtered Zr(OH)₄ was dried at 110° C. overnight and then crushed to >230 mesh (<63 μm). The xWZr supports, where x corresponds to the wt % WO₃ loading on ZrO₂, were prepared by wetness impregnation of the synthesized Zr(OH)₄ solids. 2.0 g of Zr(OH)₄ was impregnated with an ammonium metatungstate hydrate solution (0.042 M and 0.08 M for 15% and 25% WO₃ loadings, respectively), dried in air at 110° C., and then calcined at 800° C. for 3 hr (2° C./min ramp) in static air. Samples with a WO3 loading of 15 and 25 wt % on ZrO2 were prepared.
b-4. Synthesis of Al-SBA-15

The Al-SBA-15 (Si:Al=25) catalyst was prepared via impregnation. SBA-15 was calcined at 350° C. for 4 hr (1° C./min ramp) prior to use. 2.0 g of the calcined SBA-15 was then impregnated with a 0.26 M solution of AlCl₃ (>99%) in ethanol (200 proof) on a petri dish. The impregnated material was dried in air at room temperature for 12 hr followed by drying in air at 80° C. for 12 hr and then calcined at 540° C. for 4 hr with a 1° C./min ramp.
b-5. Synthesis of HBEA zeolite (Ni/HBEA)

Nickel nitrate hexahydrate (Aldrich, Ni(NO₃)₂·6H₂O, ≥99.999% trace metal basis) and BEA(25) (Zeolyst, CP814E) were used as precursors for the synthesis of Ni/HBEA catalysts. The BEA(25) support was calcined prior to use; zeolites were calcined at 550° C. for 4 h (2° C./min). Ni/HBEA catalysts was synthesized via wetness impregnation, dried in air at 110° C. overnight, and then calcined in air at 550° C. for 2 h (2° C./min). The catalysts were then reduced in 100 mL/min equimolar flow of H₂ and He at 350° C. for 2 h (10° C./min).

c. Reactor Setup
c-1. Microwave cavity description and temperature measurements

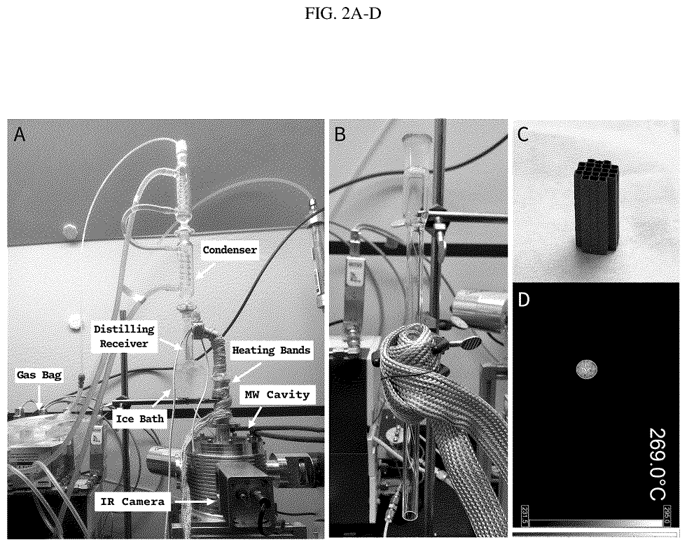

A monomode cylindrical microwave resonator with a 104.92 mm diameter and 85 mm height, manufactured at the ITACA Institute at Valencia, was used. The microwave system operated in the TE111 mode with a constant delivered power of 132 W. The operating frequency sweeped around 2.45 GHz with an adjustable frequency span (0.2-100 MHZ). The sample temperature was controlled by changing the antenna's coupling position and adjusting the frequency span. A nearly uniform electromagnetic field formed in a region of 15-mm height and 10-mm diameter, at the center of the microwave cavity, where all samples are placed within a quartz tube (10 mm inner diameter). A porous silicon carbide (SiC) monolith with channels in the millimeter range was used as a microwave susceptor (FIG. 2C). A pyrometer (CT laser LT, Optris, −50-975° C.) and an infrared (IR) thermal camera (PI 1 M, Optris, 450-1800° C.) were attached to the microwave cavity, enabling temperature measurement of the quartz reactor wall and the axial temperature profile, respectively. The IR camera was also used to monitor the formation of any localized hotspots during the reaction. Additionally, a fiber optic temperature sensor (FISO Technologies Inc., −40-300° C.) was used to measure internal temperature at the monolith walls and quantify the temperature differences between the walls of the reactor and the monolith. The pyrometer temperature reading was used as the primary reference temperature throughout the examples of the present disclosure.
c-2. Microwave pyrolysis

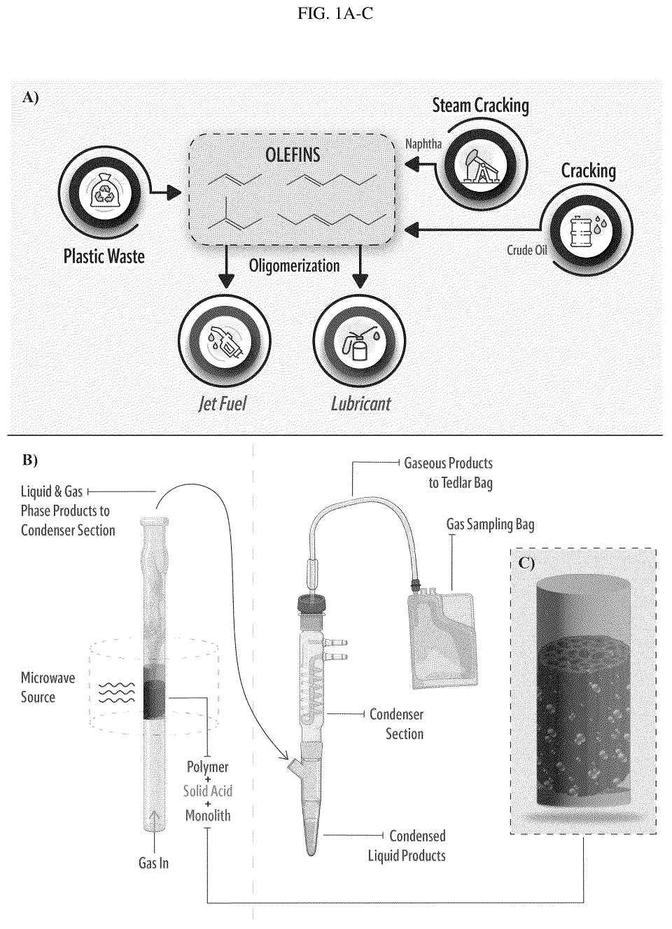

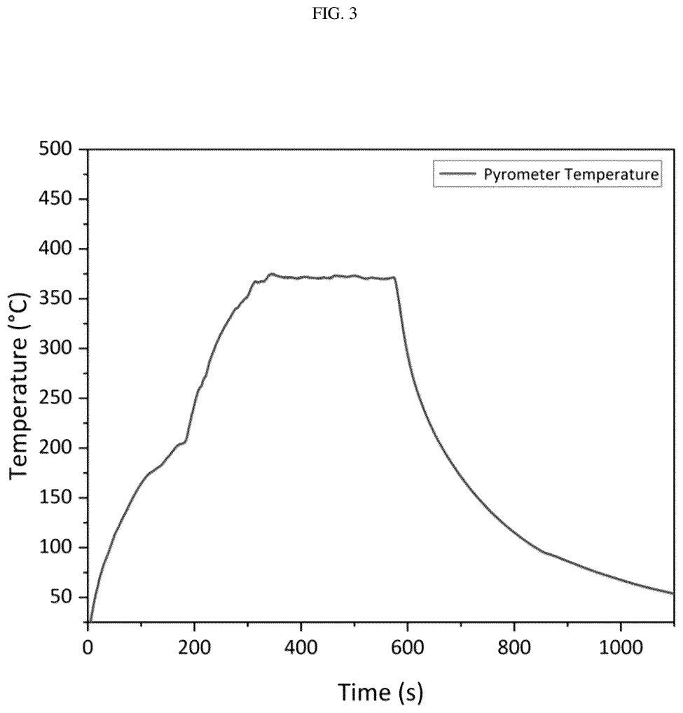

All reactions were conducted in a tubular reactor (diameter=10 mm) with a fixed porous quartz frit at the center of the tube (FIG. 1B and FIG. 2A). The samples consisting powdered LDPE mixed with solid acid were placed in and on a SiC monolith at the center of the tube (FIG. 1B and FIG. 2A). Prior to an experiment, the reactor was purged with H₂ or N₂ to remove the air. The gas flow rate was controlled using a mass flow controller (Brooks Instrument). The reactor was placed in the microwave cavity such that the monolith was at the center of the aforementioned nearly-uniform electromagnetic field region. The remainder of the tube outside the microwave cavity was wrapped with a heating band heated to 140° C. to prevent the condensation of the product in the tube. Typically, 1.0 g of LDPE and 100 mg of the catalyst were added to the reactor. For experiments with 0.5 Pt-HY (30) and Pt-SiO₂, the catalyst was reduced at 250° C. for 2 hr (10° C./min ramp) in a 100 mL/min equimolar flow of H₂ and He gas prior to use. The reactor was heated gradually to 190° C. (<1° C./s) to ensure good mixing of the polymer melt and rapidly (heating rate=2-2.5° C./s) to the reaction setpoint (375° C.) and maintained for typically 200 s (FIG. 3). A distilling receiver cooled to 0° C. using an ice bath was attached to the exit end of the reactor. The distilling receiver was combined with a condenser section cooled to 6° C. to separate liquid products from gaseous hydrocarbons. A Tedlar bag was connected to the rear end of the condenser section for the online collection of the gaseous hydrocarbons produced. Upon completion, the reactor was cooled quickly to room temperature, and the liquid products were extracted from the distilling receiver and the reactor using dichloromethane (DCM).
c-3. Conventional pyrolysis

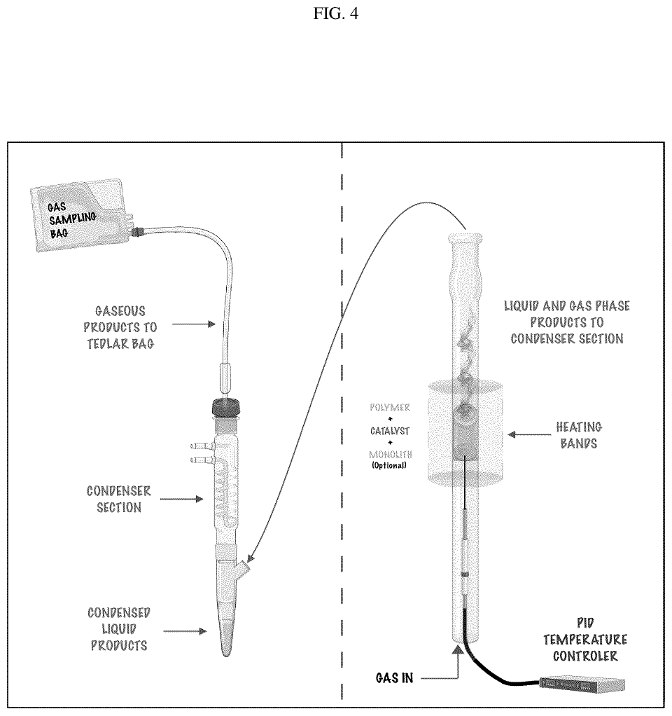

A setup nearly identical to the microwave pyrolysis was used for the conventional pyrolysis, except that high-temperature heating bands were used instead of microwaves to heat the reaction zone (FIG. 2B and FIG. 4). A thermocouple measured the internal temperature, and a PID controller was used to control the heating.

d. Catalyst Characterization

The crystalline phase identification of the catalyst was carried out by X-ray diffractometer (Bruker D8) with Cu Kα radiation (λ=1.54056 Å) at 40 kV and 40 mA and a scanning rate of 0.05 per second between 2θ=10-70°. Small-angle X-ray scattering patterns of the catalysts were recorded on a Xenocs SAXS/WAXS (Xenocs, Sassenage, France) in the range 2θ=0.02-8° using a Cu Kα radiation (λ=1.542 Å). A sample-to-detector distance of 550 mm was used. Elemental composition was analyzed using x-ray fluorescence (XRF) spectroscopy on a Rigaku WDXRF. Scanning electron microscopy (SEM) and energy-dispersive X-ray spectroscopy (EDX) analysis of the materials were performed on an Auriga 60 microscope (Carl Zeiss NTS GmbH, Germany) equipped with a Schottky field emission gun (FEG). Transmission electron microscopy (TEM) analysis was conducted using a field emission transmission electron microscope JEM-2010F FasTEM at 200 kV. N₂ physisorption at −196° C. was performed on a Micromeritics ASAP 2020 instrument. CO chemisorption was conducted in the pulse regime on an AutoChem II Micromeritics instrument. Pre-reduced samples were loaded in a quartz U-tube reactor and heated to 250° C. in the flow of 10% H₂/He for 2 hr (10° C./min ramp rate). Fourier transform infrared (FTIR) spectra of adsorbed pyridine followed by pyridine thermodesorption were recorded in transmission mode in a homemade pyrex tubular flow cell equipped with 32 mm KBr windows. The sample was pressed in a self-supported wafer (˜15 mg, 1.3 cm² and 40 bar/cm² pressure), placed in a quartz sample holder, and heated in flow of pure Ar at 300° C. (ramping rate 10° C./min) with 1 h dwell at that temperature. Then, the temperature was reduced to 150° C., and the sample was treated with pyridine vapour by injecting liquid pyridine (5 μl, 99.8%; Sigma-Aldrich) with a micro syringe through a septum port. After saturation, the sample was flushed with pure He for 30 min, and the spectrum of pyridine-saturated sample was recorded. Finally, the temperature was increased with a 10° C./min rate to 300° C. in constant flow of Ar, and spectra were recorded every 1 min. Integration and peak deconvolution were done using the Omnic 8.2 software.

e. Product Analysis

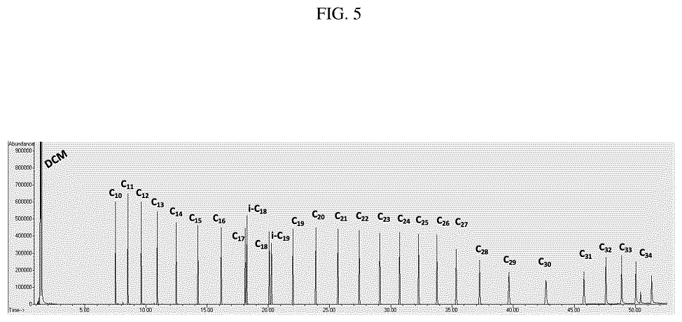

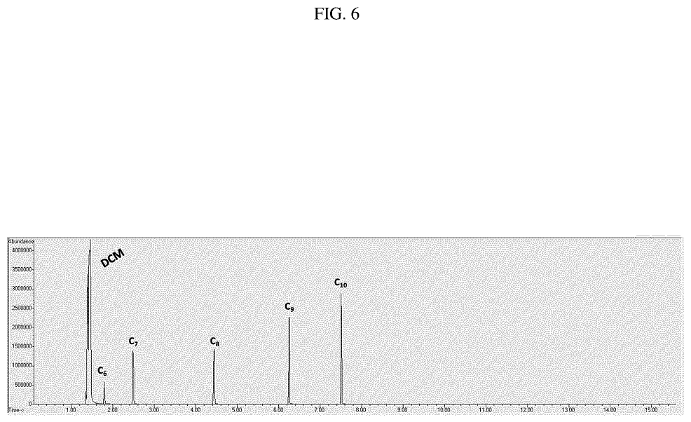

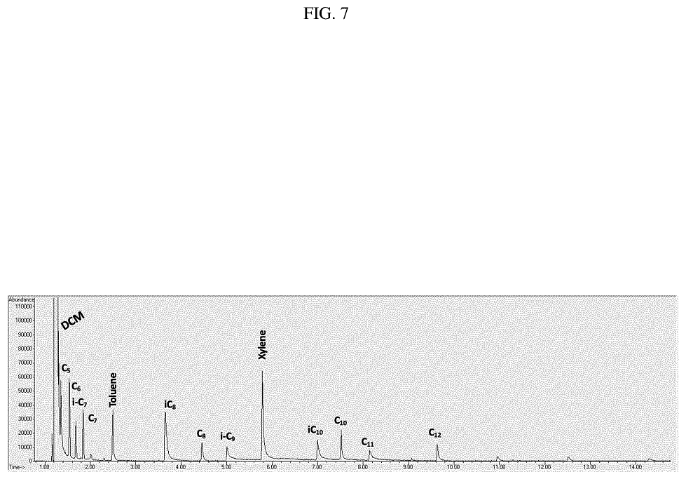

Gaseous products formed during the reaction were collected in a Tedlar gas sampling bag and analyzed with a GC-FID (Agilent HP-PLOT/Q GC column). The residual oil mixture was extracted by addition with ˜20 mL of CH₂Cl₂. ˜20 mg of n-octacosane was added as an internal standard and the solution was mixed on a vortex mixer until the n-octacosane was completely dissolved. The resulting solution was analyzed by GC-FID (Agilent HP-1 column) for product quantification and GC-MS (Agilent DB-5 column) for product identification after filtration with a syringe filter. Calibration coefficients and retention times for all products were measured using C₁-C₃₅ analytical standards (FIGS. 5-7).

The conversion of LDPE was calculated as follows:

$$LDPE Conversion ( \% ) = \frac{{W}_{Rxtr , i} - {W}_{Rxtr , f}}{{W}_{LDPE , i}} \times 100 \%$$

where W_{Rxtr,i} is the initial weight of the reactor with polymer, catalyst, and the monolith, W_{Rxtr,f} is the final weight of the reactor, and W_{LDPE,i} is the initial weight of the LDPE used. The alkane/olefin selectivities were calculated as follows:

$$Olefin selectivity ( \% ) = \frac{Molar Yie1d of Olefins}{Overall Yield of Products} \times 100 \%$$

$$Molar Yie1d of Olefins = {\sum }_{n = 2}^{17} ( Yield of {C}_{n} Olefins )$$

$$Overall Yield of Products = {\sum }_{n = 1}^{17} ( Yield of {C}_{n} Alkanes ) + {\sum }_{n = 2}^{17} ( Yield of {C}_{n} Olefins ) + \sum ( Yield of Aromatic Products )$$

The yields of Cₙ alkanes, Cₙ olefins and aromatics were quantified using GC-FID.
f. Coke Characterization of Spent Catalysts

The coke samples were collected by flushing the reactor with DCM, followed by drying the samples in air at 70° C. TGA and DSC analyses were performed on a Q600 SDT (TA instruments). Raman spectra of coke samples were recorded under ambient conditions on a Horiba LabRam microscope with a 15× objective using a 325 nm UV laser. The deconvolution of the spectra was done using the OMNIC software.

g. Temperature Profile Measurements

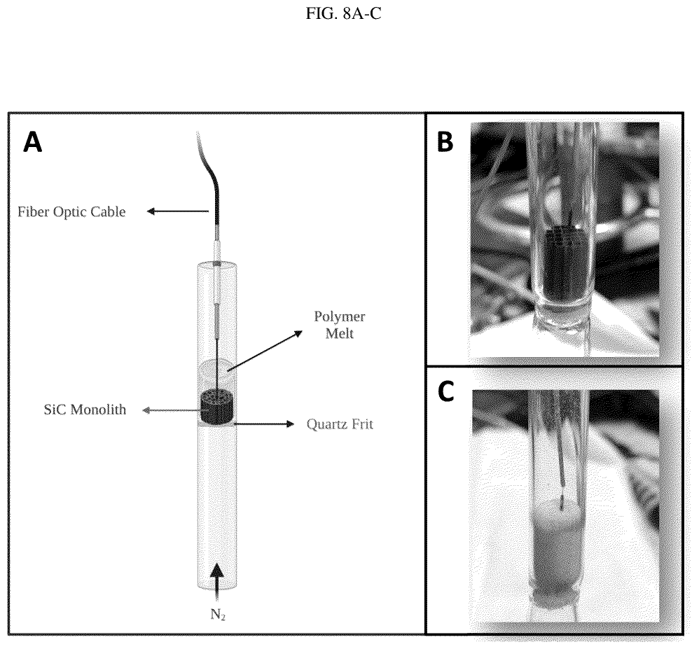

Temperature measurements at different flow conditions (3-150 mL min⁻¹) were conducted at the center of the monolith (using a fiber optic temperature sensor) and the walls (using a pyrometer). Various filler materials (no filler, quartz, and LDPE) were used to eliminate the void between the wall and the monolith. An experimental setup described in FIG. 8A-C was used. Additionally, an IR camera was used to scan for hotspots during MW heating.

h. COMSOL Simulations of MW Heating

Modeling structured reactors in a microwave cavity required the investigation of multiple coupled phenomena: the electromagnetic field and the dissipation of the electromagnetic energy in the solid, the fluid flow through the reactor, and the thermal transport in each phase and between phases. A thermal conductivity (˜100 W/m-K) and dielectric properties (9.8-1.1 j) of SiC from the literature was used in the examples of the present disclosure (See A. Malhotra, et al., Temperature Homogeneity under Selective and Localized Microwave Heating in Structured Flow Reactors, Industrial & Engineering Chemistry Research, 60(18) (2021) 6835-6847; or H. Goyal, et al., Scaleup of a Single-Mode Microwave Reactor, Industrial & Engineering Chemistry Research, 59(6) (2019) 2516-2523). The steady-state heat transfer equation was solved after solving the electric field with a heat generation term from the dissipation of the electric field (MW heating) was given as:

$${\dot{Q}}_{j , emw} = 0.5 σ {\vert E \vert}^{2} + π {ε}_{o} {ε}_{c} {\vert E \vert}^{2}$$

where E is the electric field vector, fis the frequency of the microwave radiation, ϵ₀ is the permittivity of vacuum, ϵ_{c} is the complex part of the relative permittivity, and σ is electrical conductivity. E was obtained by solving Maxwell's equations using the impedance boundary conditions for the metallic cavity to minimize the computational load. A coaxial port was used to supply the energy to the cavity. First-order scattering conditions were used at the inlet and outlet of the quartz tube to avoid reflection artifacts. The reactor tube was exposed to room temperature, where it losed heat to the ambient through Newton's law of cooling (See R. B. Bird, et al., Transport phenomena, 2nd, Wiley international ed. ed., J. Wiley, New York, 2002). To determine the role of gas flow rate in the temperature difference, the Navier-Stokes equation was solved to determine the flow field in the reactor in the laminar flow regime established at low flow rates analyzed here. The flow profile was solved iteratively with the thermal transport. The effect of different flow rates was established through the average Nusselt number that increases as the flow velocity increased. A domain with effective thermal properties was simulated in the region between the wall and the monolith to determine the role of filler properties.
i. Process Modelling, Techno-Economic and Life Cycle Analyses

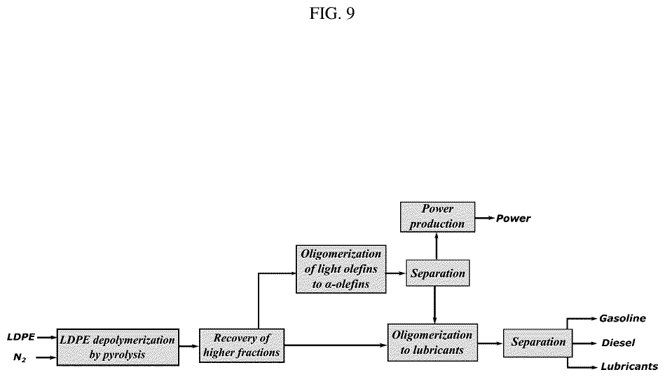

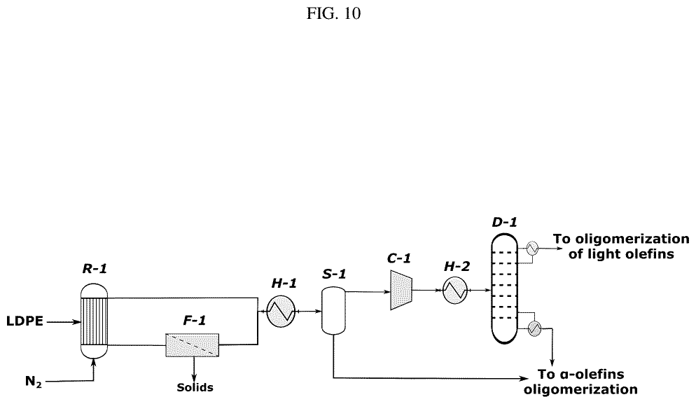

The economic potential of MW slurry technology was evaluated considering the production of lubricants from olefins to allow comparison with the market. The integrated process designed to produce lubricants from LDPE via pyrolysis comprised of the stages presented in FIG. 9. The process was modelled using Aspen Plus® v.11 and defined in more detail in FIG. 10 to FIG. 12. The representative components of the paraffins and olefins were in Table 1.

TABLE 1

| | REPRESENTATIVE COMPOUND | |
|---|---|---|
| Carbon Number | Alkane | Alkene |
| 1 | Methane | — |
| 2 | Ethane | Ethylene |
| 3 | Propane | Propene |
| 4 | Butane | 2-methyl prop-1-ene |
| 5 | Pentane | 2-methylbut-1-ene |
| 6 | 2-methyl Pentane | 2-methyl-pent-2-ene |
| 7 | 2-methyl hexane | 2-methyl-hex-en |
| 8 | 2-methyl heptane | Oct-4-ene |
| 9 | 2-Methyl Octane | Non-4-ene |
| 10 | 2-Methyl Nonane | Dec-5-ene |
| 11 | 2-Methyl Decane | Undec-5-ene |
| 12-19 | Pentadecane | Pentadec-7-ene |
| C6-C10 fraction of light | | Oct-4-ene |
| olefin oligomerization^{a} | | |
| Lubricant fraction^{b} | | Triacontene |

^{a}Oligomerization is assumed to stop in the first step of the oligomerization reported in C. S. Hsia-Chean, et al., Production of lubricant range hydrocarbons from light olefins, U.S. Pat. No. 4,568,786 (709143) (1986).
^{b}Taken from the most representative component of commercial lubricants analysed in C. Yang, et al., Oil fingerprinting analysis using gas chromatography-quadrupole time-of-flight (GC-QTOF), Standard Handbook Oil Spill Environmental Forensics: Fingerprinting and Source Identification, 2nd Edition (2016) 449-480.

Most of their properties were obtained from the Aspen database. Only LDPE were modelled by defining the structure of the molecule with the Joback method and using the reaction enthalpy (See P. L. Splitstone, et al., The Enthalpies of Combustion and Formation of Linear Polyethylene, Journal of Research of the National Bureau of Standards—A. Physics and Chemistry, 78A(5) (1974) 611-616), heat capacities (See S. S. Chang, Heat capacities of Polyethylene from 2 to 360 K. I Standard Samples of Linear and Branched Polyethylene Whole Polymer, Journal of Research of the National Bureau of Standards—A. Physics and Chemistry 77A(4) (1973) 395-405), and heat of fusion (See Broadhurst, Thermodynamic Properties of Polyethylene Predicted from Paraffin Data, Journal of Research of the National Bureau of Standards—A. Physics and Chemistry, 67A(3) (1963) 233-240) reported by the National Bureau of Standards for branched polyethylene.

Techno-economic (TEA) and life cycle analyses (LCA) were performed to evaluate the economic potential and environmental impacts of MW slurry technology. The plant operates 8,000 h/y and treats 32,000 Mt/y of clean LDPE. The capacity corresponds to 1% of the average LDPE produced in the United States per year during the last decade (3.208 MMt/y, see Tiseo, Y, Low density polyethylene production in the United States from 1990 to 2019). The value is also in the range of industrial recycling facilities reported in other works (See M.

Larrain, et al., Techno-economic assessment of mechanical recycling of challenging post-consumer plastic packaging waste, Resour Conserv Recy 170 (2021)). TEA and LCA have been performed for the case studies presented in Table 2 with the aim of determining the influence of conventional vs. MW slurry reactors, and the effect of using catalysts with different selectivity to olefins.

TABLE 2
Cases evaluated in the TEA and LCA studies.

| Case Study | Description of the technology |
|---|---|
| Conv | LDPE pyrolysis with conventional heating. |
| Monolith-Pt | LDPE pyrolysis with conventional heating and |
| | a monolith with 0.5Pt-HY(30) for improving |
| | the heat transfer. |
| Monolith-Al | LDPE pyrolysis with conventional heating and |
| | a monolith with Al-SBA-15 for improving |
| | the selectivity to olefins and the heat transfer. |
| MW-Pt | LDPE MW pyrolysis on 0.5Pt-HY(30). |
| MW-Al | LDPE MW pyrolysis on Al-SBA-15. |

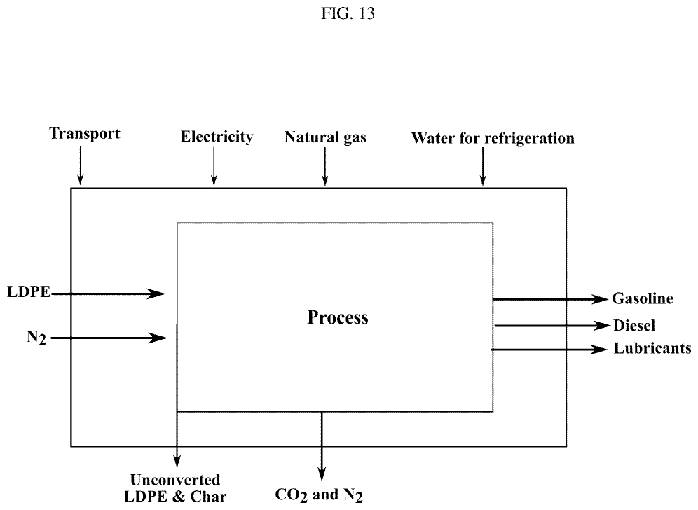

Note that the use of hydrogen as a heat transfer carrier is not included since it is more expensive than nitrogen and lower conversion to olefins is obtained. LCA was performed to evaluate the environmental performance of MW slurry versus conventional heating in the production of lubricants. System expansion method is used for estimating the emissions of the process, simplified as in FIG. 13. One kg of lubricant is considered a functional unit for the analysis and comparison with oil-based lubricants from Ecoinvent database v3.8 (See E. Santos, et al., A catalytic reactive distillation approach to high density polyethylene pyrolysis - Part 1 - Light olefin production, Chemical Engineering Journal 378 (2019) 122077).

### Example 1. Optimization of Reaction Parameters

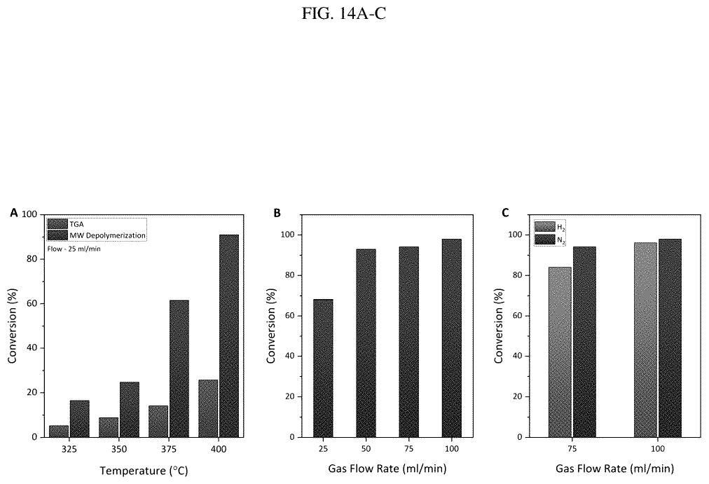

The reaction temperature and bubbling gas flow rate were optimized (FIG. 14A-C) at 375° C. and 100 ml min⁻¹ of N₂, achieving >96% conversion over zeolite-Y (HY) in 200 seconds. In the absence of HY, no activity was observed below 375° C. (according to TGA; FIG. 14A). Hence, the solid acid clearly promotes extensive low-temperature cracking. Supporting Pt on the HY (Table 3) slightly increased the alkanes yield due to the Pt's ability to hydrogenate olefins; however, this effect was negligible (difference in olefin selectivity <5%), possibly due to rapid coking of Pt. Catalytic pyrolysis over Pt-SiO₂ catalyst demonstrated negligible LDPE conversions (<7%), ruling out hydrogenolysis as a pathway over Pt-HY under these short contact times.

TABLE 3
Results for pyrolysis of LDPE over 0.5Pt-HY, 1.0Pt-SiO2, and Al-SBA-15 at
different gas flow conditions with MW heating and CH. Reaction conditions:
Temperature - 375° C., Gas Flow Rate - 100 ml min-1 and Time - 200 seconds.

| | | | LDPE | | | |
|---|---|---|---|---|---|---|
| | Heating | | Conversion | Product Selectivity (%) | | |
| Catalyst | Mode | Gas | (%) | Alkanes | Olefins | Aromatics |
| 0.5Pt-HY | MW | H₂ | 96.4 | 71.71 | 24.70 | 3.57 |
| 0.5Pt-HY | MW | N₂ | 96.1 | 59.61 | 37.72 | 2.67 |
| 0.5Pt-HY | CH | N₂ | 13.4 | 86.74 | 12.8 | 0.46 |
| 0.5Pt-HY | CH- | N₂ | 45.5 | 69.42 | 26.84 | 3.74 |
| | Monolith | | | | | |
| 0.5Pt-HY | CH- | N₂ (3 ml | 14.2 | — | — | — |
| | Monolith | min⁻¹) | | | | |
| 1.0Pt-SiO₂ | MW | H₂ | 7.5 | 67.18 | 32.08 | 0.74 |
| Al-SBA-15 | MW | H₂ | 90.6 | 12.43 | 87.45 | 0.12 |
| Al-SBA-15 | MW | N₂ | 89.7 | 12 | 87.9 | 0.1 |
| Al-SBA-15 | CH- | H₂ | 68.16 | 24.4 | 75.42 | 0.18 |
| | Monolith | | | | | |

### Example 2. Evaluation of Catalyst and Reactivity

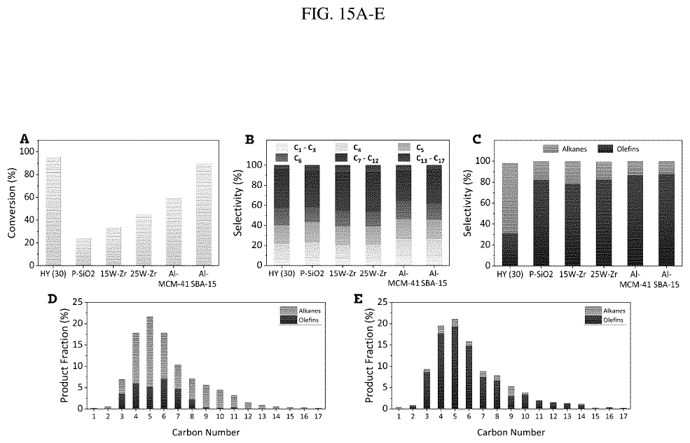

MW pyrolysis over several acid catalysts results in C₄-C₁₂ hydrocarbons (FIG. 15B, D-E). The conversion follows the order: P-SiO₂<15WZr<25WZr<Al-MCM-41<Al-SBA-15<HY (FIG. 15A). The reactive distillation narrows the product molecular weight distribution and effectively separates products up to the C₉ (the yield of >C₁₀ hydrocarbons is low). Heavier hydrocarbons undergo back-mixing and further cracking because the heating bands reach only 140° C., lower than their boiling point. This distribution is expected to be tuneable by changing the distillation unit (reactor exhaust) temperature; heating bands are not necessary in large scale where heat losses are small, and systems can operate close to adiabatic. The catalytic activity originates from an interplay between acid site density, acid strength, and porosity. For example, the Bronsted Acid Site (BAS) density of P-SiO₂ decreases steeply from 30 μmol/g (300° C.) to 4 μmol/g (375° C.), whereas Al-MCM-41 and Al-SBA-15 show a more gradual decrease from ˜60 μmol/g to ˜40 μmol/g. Hence, the lower activity of P-SiO₂ is related to its weak acid sites. On the other hand, Al-MCM-41 and Al-SBA-15 show higher activity as they have a higher density of BAS and their acid sites possess higher intrinsic strength (Table 4).

TABLE 4
Textural properties of different catalyst samples.

| | BET Surface | | | Brønsted | Lewis acid |
|---|---|---|---|---|---|
| | Area | V_{micro}^{a} | Vₘₑₛₒ^{b} | acid sites | sites |
| Sample | (m²/g) | (cm³/g) | (cm³/g) | (μmol/g)^{c} | (μmol/g)^{c} |
| HY (30) | 566 | 0.29 | 0.28 | 320 | 100 |
| 0.5Pt-HY(30) | 555 | 0.29 | 0.28 | — | — |
| P—SiO₂ | 323 | — | 0.52 | 30 | 0.22 |
| 15WZr | 54 | — | 0.15 | 30 | 37 |
| 25WZr | 54 | — | 0.15 | — | — |
| Al-MCM-41 | 881 | — | 0.78 | 63 | 74 |
| SBA-15 | 435 | — | 0.52 | — | — |
| Al-SBA-15 | 303 | — | 0.46 | 53 | 140 |
| Al-SBA-15^{#} | — | — | — | 50 | 123 |
| HZSM-5 | 249 | 0.13 | — | 246 | 13 |

^{a}obtained from t-plot results;
^{b}obtained from BJH desorption results;
^{c}measured using FTIR of adsorbed pyridine;
^{#}Spent catalyst after calcination in static air at 550° C.

The differences between Al-MCM-41 and Al-SBA-15 are likely due to the differences in their pore diameters ˜3.4 nm for Al-MCM-41 vs. ˜5.5 nm for Al-SBA-15 (obtained using N₂ physisorption). Post-characterization of the spent Al-SBA-15 catalyst further revealed that the BAS density does not change in the spent catalyst after calcination in static air at 550° C. (Table 4), suggesting that the catalyst is robust and reusable.

Reactions over HY generated primarily mono-and di-branched alkanes (˜70%) as the major products (FIG. 15B). On the contrary, mesoporous materials have larger pore sizes (˜2-50 nm) and low to moderate acid strengths. Interestingly, they produced linear or mono-branched olefins with internal C═C bonds at a high selectivity (79-88%; FIG. 15C). P-SiO₂ and WZr produced a slightly higher C₇-C₁₇ fraction due to their lower density or weaker Bronsted acid sites than the rest. FTIR spectroscopy of adsorbed pyridine coupled with thermodesorption confirm that P-SiO₂ possess fewer and weaker Bronsted acid site. The olefin selectivity follows Al-SBA-15>Al-MCM-41>xWZr>P-SiO₂>>HY(30).

### Example 3. Transient LDPE MW-slurry Pyrolysis

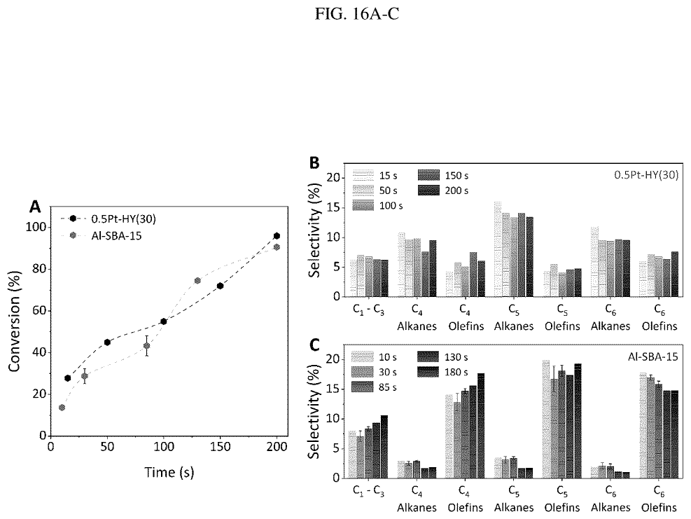

Contact time experiments over HY and Al-SBA-15 showed ˜96% and ˜90% conversions in 200 s. LDPE conversion increases monotonically with reaction time, while the product distribution does not change considerably (FIG. 16A). C₄-C₁₂ hydrocarbons are the major products; the cracking of longer hydrocarbons is faster, resulting in secondary cracking to lighter products. Al-SBA-15 forms lighter fractions (C₁-C₄) with increasing time at the expense of medium-sized fractions (C₅-C₇), suggesting secondary cracking (FIG. 16C). HY does not exhibit such a phenomenon and results in kindred product distributions, likely due to the rapid coking of its acid sites. This was further corroborated by a decrease in C₄-C₆ hydrocarbons after 15 s (FIG. 16B).

### Example 4. Conventional vs. Microwave Pyrolysis

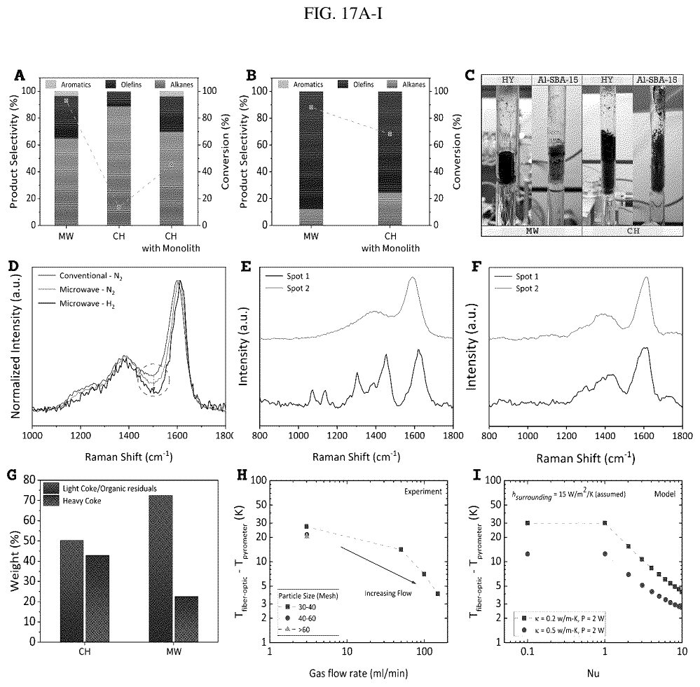

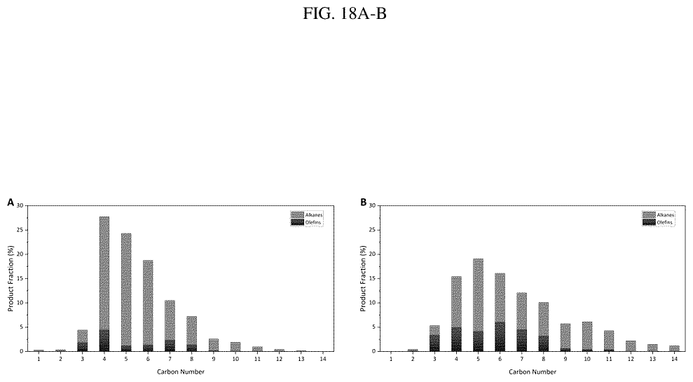

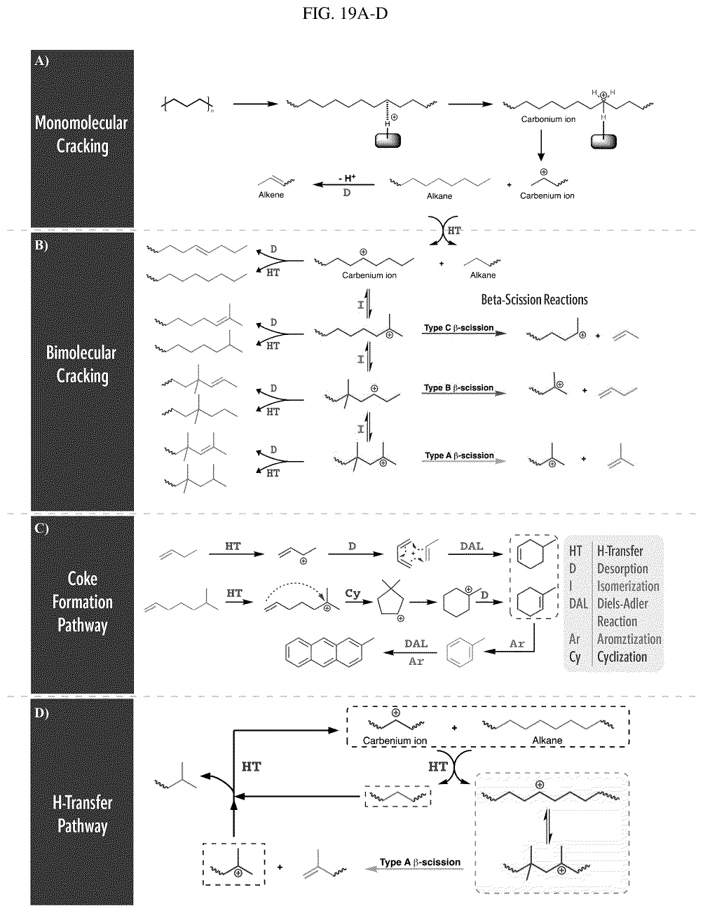

MW and CH pyrolysis revealed notable differences in the LDPE conversion (96% vs. only 46%; FIG. 17A-B). In addition, CH pyrolysis without a monolith resulted in even lower conversion (13%), and the product mainly contained light C₄-C₆ alkanes (FIG. 17A and FIG. 18A). With a monolith, the distribution shifted to C₆-C₁₂ with ˜30% olefins (FIG. 18B). Heat delivery (MW vs. CH) impacts the thermal gradients in slurry pyrolysis. Excessive thermal gradients in CH, created by hot zones on the sidewalls of the reactor, lead to coke formation (FIG. 17C) and catalyst deactivation via forming heavy aromatics due to rapid H-transfer at these hotspots. These polyaromatics cause significant pore blockage, enhance the hydrocarbons' residence times in the zeolite pores, and promote secondary cracking to light products (FIG. 19C). Such effects have previously been reported in fixed bed reactors for methanol-to-olefin reactions (See S. V. Konnov, et al., Mechanism of SAPO-34 catalyst deactivation in the course of MTO conversion in a slurry reactor, Catalysis Science & Technology 8(6) (2018) 1564-1577). In addition, CH pyrolysis exhibits higher alkane selectivities than MW-assisted pyrolysis, further indicating that CH intensifies H-transfer reactions leading to coke (Table 3) due to improved heat transfer in the slurry than in a fixed bed. The monolith distributes the gas and reduces thermal gradients due to efficient heat transfer from the walls to the center of the reactor.

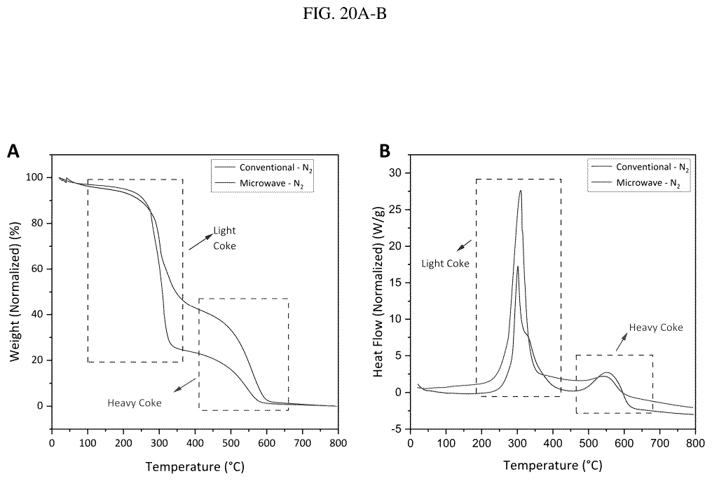

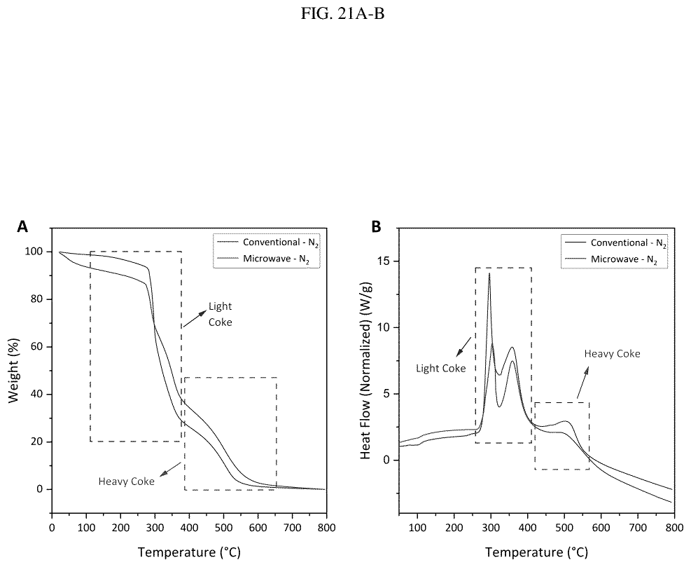

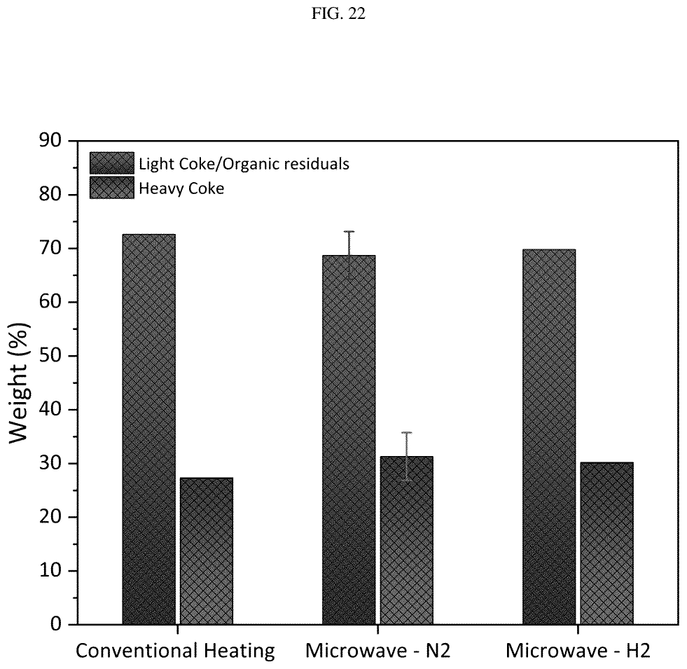

While both heating modes generate coke, MWs generated less coke over both HY and Al-SBA-15 catalysts (FIG. 17C), reflected in the LDPE conversions. Thermogravimetric analysis (TGA) and differential scanning calorimetry (DSC) of coked HY indicate two distinct bands for light coke (or organic residuals; centered at ˜295° C.) and one for heavy coke (centered at ˜560° C.) (FIG. 20A-B). The maxima shifts by ˜10° C. for CH pyrolysis (FIG. 20A-B). The maxima of the Differential Thermal Analysis (DTA) curves are in the order of MW-H₂<MW-N₂<CH-N₂. Al-SBA-15 exhibits two bands for light coke (centered at ˜290° C. and ˜350° C.) and one for heavy coke (centered at ˜500° C.) (FIG. 21A-B). Furthermore, the heavy-coke band of HY is centered at ˜560° C. compared to Al-SBA-15 (˜500° C.), suggesting heavier polyaromatics in HY. The TGA weight loss (FIG. 17G) indicates that over HY catalyst the MW pyrolysis exhibits ˜49% less heavy coke. On the other hand, MW and CH samples over Al-SBA-15 show only ˜30% heavy coke; the rest is light coke (FIG. 22). This result further rationalizes the smaller difference in the conversion of MW and CH over Al-SBA-15 than HY, suggesting that the former is not affected by heavy coke as much as the latter due to its large pores that are less prone to H-transfer and allow for faster diffusion of larger hydrocarbons.

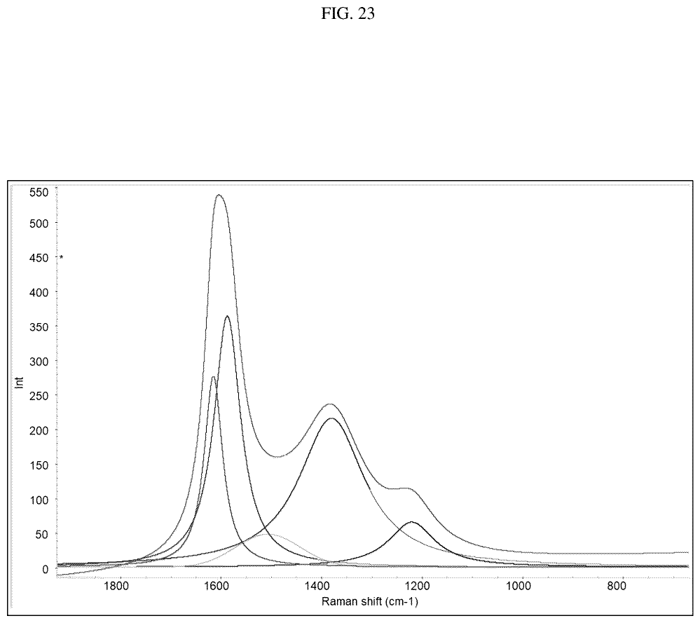

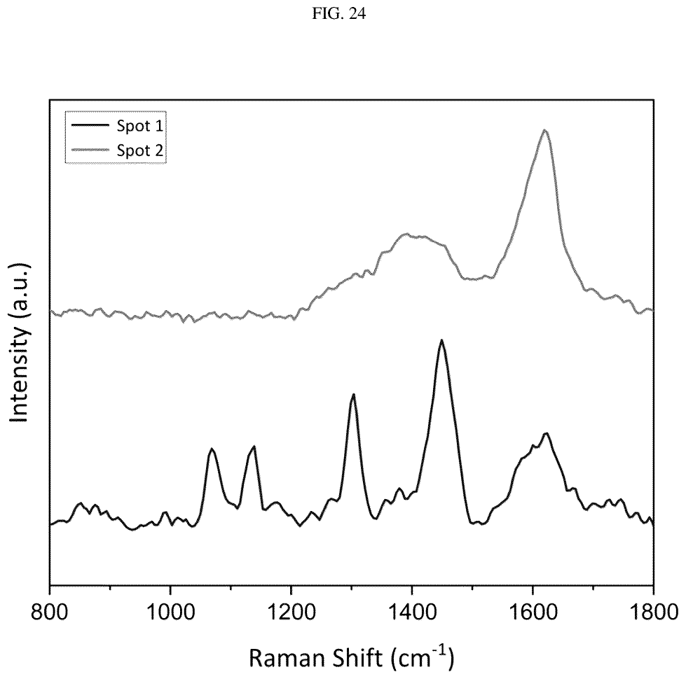

FIG. 17D shows the Raman spectra of the coke on HY catalyst. The coke in CH-N₂, MW-N₂, and MW-H₂ is graphitic carbon, indicated by the intense G band centred at 1600 cm⁻¹. The greater intensity of the D₃ band (higher I(D₃)/I(G) ratio) of the coke-rich CH sample (FIG. 23) corresponds to amorphous carbon arising from polycyclic aromatics. The I(D₃)/I(G) ratio of the MW samples in different gas flows implied a lower I(D₃)/I(G) in H₂, suggesting that hydrogen hydrogenates coke precursors in HY and reduces polyaromatic coke (Table 3). The DTA analysis further confirmed this, wherein the MW-H₂ sample corresponded to the lowest temperature amongst the three samples. FIGS. 17E-F and FIG. 24 show Raman spectra of the coked Al-SBA-15 with CH and MW captured using a microscope at different spots in the same sample. CH samples have similar spectra (FIGS. 17 E-F) due to uniform coverage with graphitic coke. For MW samples (FIG. 17F, FIG. 24) at spot 1 (FIG. 17E-F), the coke is similar to HY (FIG. 17D), with an intense G band suggesting graphitic coke. At spot 2 (FIG. 17F, FIG. 24), characteristic features at 1065, 1130, 1170, 1296, 1370, 1440 cm⁻¹ correspond to unreacted polyethylene. This shows that PE is slowly pyrolyzed in SBA-15, and the new peak in TGA/DSC corresponds to residual polymer rather than coke. The large pores of SBA-15 allow for easy access of polymers to acid sites resulting in softer depolymerization. On the contrary, the coke and light alkane formation over HY is governed by H-transfer, driving rapid coke formation and subsequent pore blockage.

### Example 5. Thermal Gradients and Coke Formation

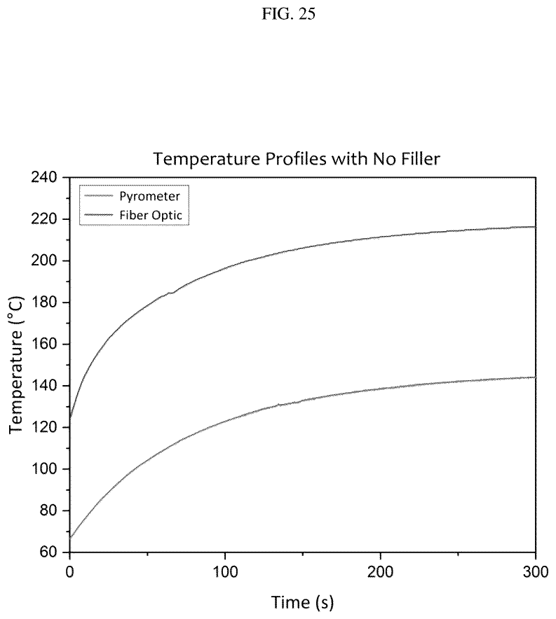

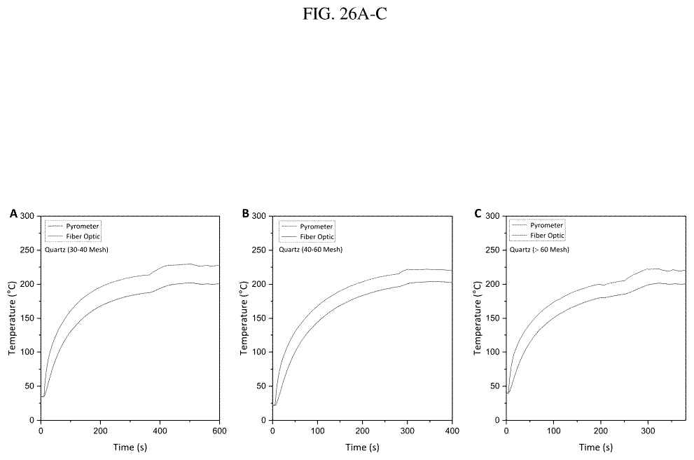

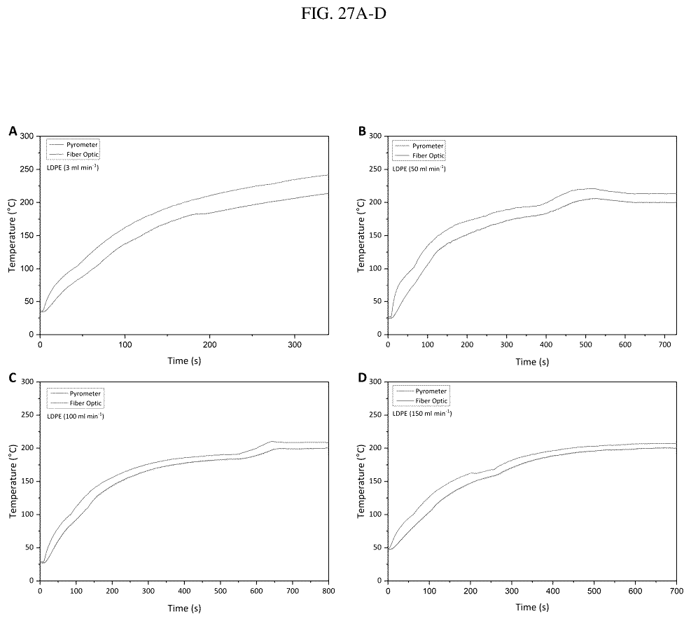

Non-isothermal operation of reactors can expedite catalyst coking. CH and MW pyrolysis behave differently mainly due to H-transfer mediated coking (FIG. 17A-B), which is more pronounced in the zeolite than Al-SBA-15. Volumetric MW heating and mixing minimize thermal gradients. Therefore, the temperature profile was assessed during the MW heating using a setup described in FIG. 8A-C. Temperature measurements showed a significant difference between the wall and the monolith center (˜60° C.) without any filler (FIG. 25). When quartz particles were used as filler, this difference reduced to ˜20-30° C. (FIG. 17H, FIG. 26A-C), and became smaller with finer quartz particles (FIG. 17H, FIG. 26A-C). The temperature difference was ˜20-30° C. using LDPE as a filler (FIG. 27A-D) and almost negligible (<5° C.) at high gas flow rates (≥100 mL min⁻¹). Similar behavior was observed at reaction conditions in CH pyrolysis with a monolith and significantly lower conversion was observed at low N₂ flow rates (˜3 ml min⁻¹) (Table 3), indicative of transport limitations. This suggests that the bubbling of gas through the melt enhances the heat and mass transfer and retards coke formation by reducing thermal gradients. COMSOL Multiphysics simulations showed good qualitative agreement with the experimental data and demonstrated that by increasing the material's thermal conductivity or increasing the Nusselt's number (by increasing the gas flow rate), a thermally uniform reaction domain is established (FIG. 17I). In contrast to MWs heating the SiC monolith directly, the CH reactor walls are hotter than the center to enable heat flow inwards, creating temperature gradients. This inherent difference is central to achieving a thermally uniform system and suppressing coke formation. In addition to facilitating selective heating, SiC is a high thermal conductivity material distributing heat, and its porous structure facilitates the establishment of an effective slurry. These factors help maintain near-isothermal conditions. While testing at the laboratory scale is insightful and helps in advancing the field, there are several challenges that can arise when dealing with the valorisation of real-life waste streams via pyrolysis. One such challenge is associated with the different properties, such as molecular weight, viscosity, thermal conductivity, etc. of the polymer waste stream which often contains a blend of different grades of plastics. The changes in the properties can affect the choice of process parameters. The COMSOL Multiphysics simulations showed that using polymers of different thermal conductivities can lead to significant thermal gradients (FIG. 17I). Furthermore, high Nusselt numbers (which can be achieved by operating at high flow rates) can eliminate these gradients even with heavier polymers (FIG. 17I). Moreover, using MW susceptors with enhanced penetration and heat transfer capabilities, such as foams or monoliths of good dielectric materials, and by smaller-scale modular operation, makes the scale-up of MW reactors feasible.

### Example 6. Hydrogenolysis and Hydrocracking over Ni-based Catalysts

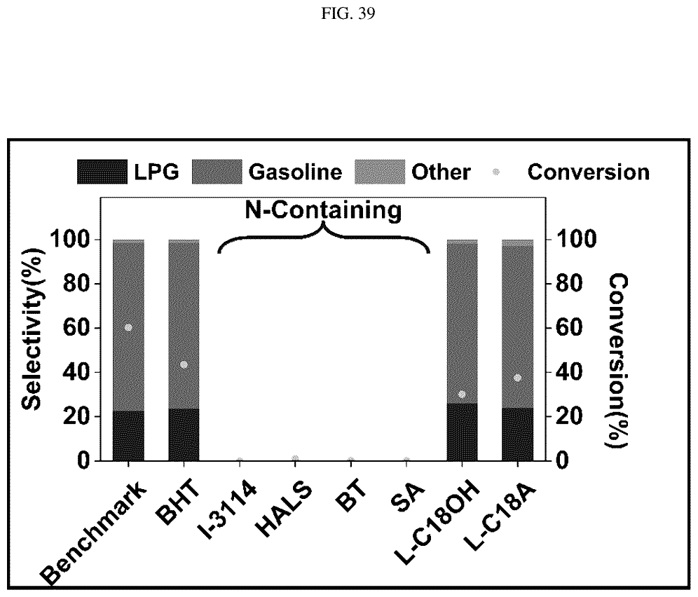

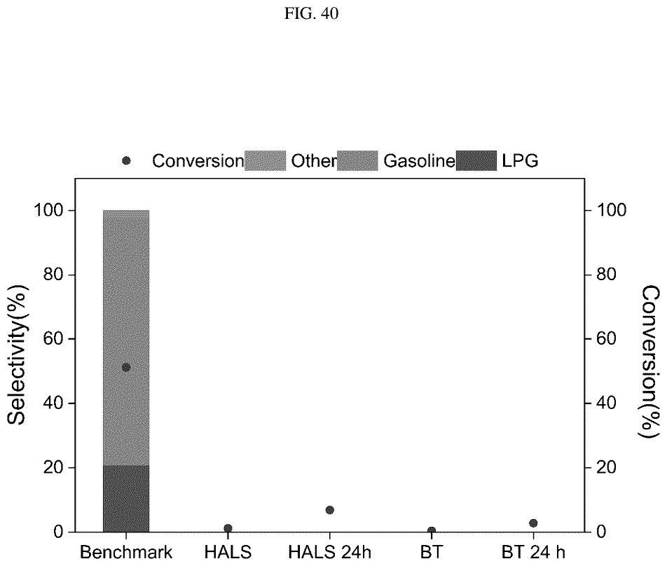

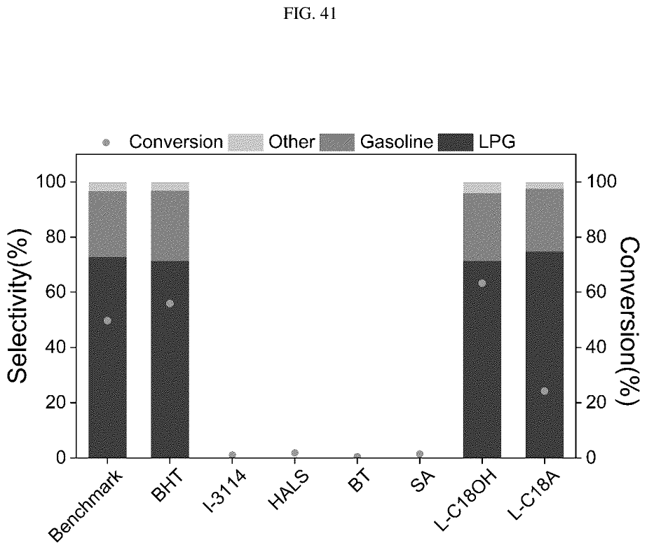

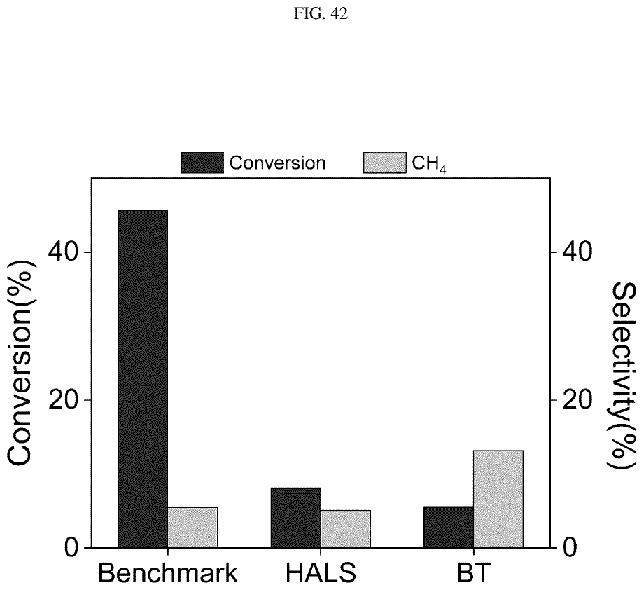

Ni supported on HBEA zeolite (Ni/HBEA) showed excellent activity for virgin 76LDPE (low-density polyethylene, molecular weight M_{w} ˜76 kDa, Sigma) hydrocracking, achieving 42% solid yield and 40% yield of C₃-C₁₂ alkanes within 1 h at 250° C., 60 bar H₂, and a polymer-to-catalyst ratio of 50 (FIG. 39). Additives at 2 wt % (20 mg) curtail conversion and increase the solid yield to varying degrees (FIG. 39). BHT, L-C18OH, and L-C18A (Table A, entries 9-11) increase solid yields by a modest 15-30% without altering product selectivity (FIG. 39). Conversely, I-3114, HALS, BT, and SA (Table A, entries 2, 5, 7, and 11) resulted in full deactivation, with 99% solid left (FIG. 39). Solid deconstruction did not occur by extending the reaction time to 24 h, confirming full deactivation of Ni/HBEA (FIG. 40). Inactivity was also observed over the hydrocracking Ni/HZSM-5 catalyst (FIG. 41) and nickel supported on silica (Ni/SiO₂), a hydrogenolysis catalyst (FIG. 42).

### Example 7. Catalytic Cracking over Solid-Acid

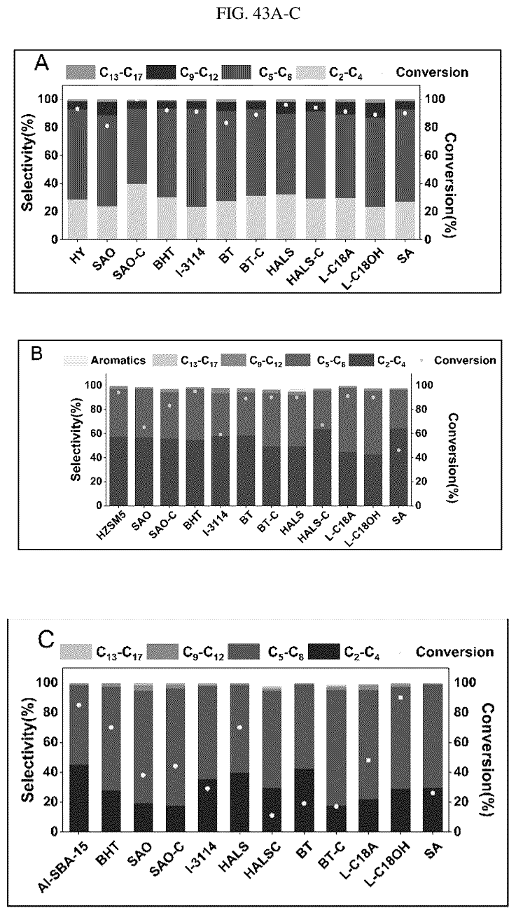

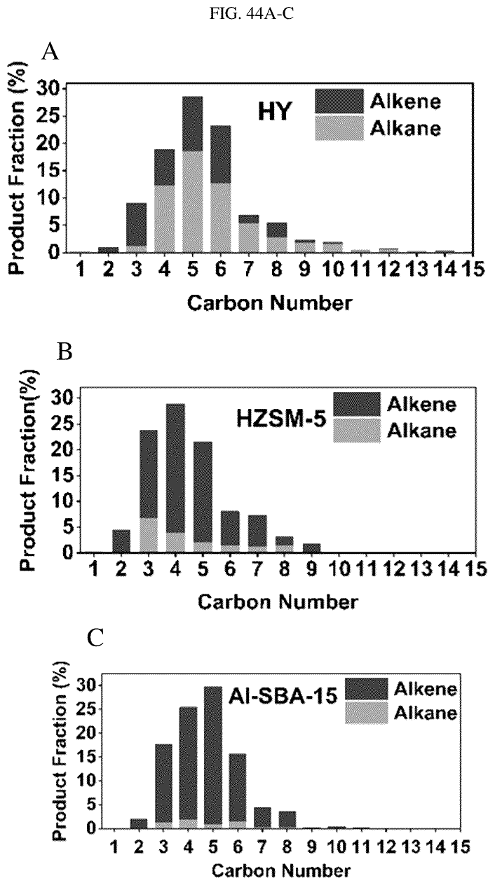

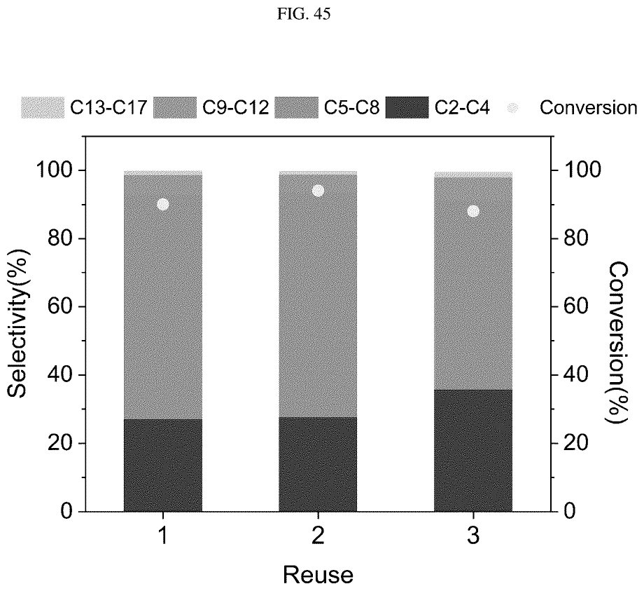

Additive-containing LDPE was catalytically cracked over three solid acid materials HY, HZSM-5, and Al-SBA-15, respectively. The reaction was carried out by employing microwave heating with a silicon carbide microwave absorber to enable fast and uniform heating and quick removal of products to avoid coking and further cracking from secondary reactions. FIGS. 43A-C shows reactivity data of LDPE with various additives under melt catalytic pyrolysis conditions over HY, HZSM-5, and Al-SBA-15. Surprisingly, these catalysts demonstrated significantly more active than hydrocracking catalysts. Cf. FIGS. 39-42 with FIGS. 43A-C. Additives minimally affected the conversion and selectivity over the HY catalyst. The product distribution consisted of C₁-C₁₂ mono and di-branched alkanes, and mainly light naphtha range (C₃-C₈), due to enhanced hydride transfer (FIG. 44A). Furthermore, the conversion and selectivity were relatively constant across three catalyst uses (FIG. 45). HZSM-5 was largely unaffected by many additives, except for SAO, I-3114, HALS-C, and SA, which lowered the conversion. The products consisted of predominantly linear and branched olefins, cyclic olefins, and monocyclic aromatics in the C₁-C₁₂ range (FIG. 44B). The conversion was relatively low for catalyst Al-SBA-15 when the polymer contained HALS-C, BT, BT-C, or SA while the conversion was greater than 60% when the additives were BHT, HALS, or L-C18OH. See FIG. 43C. The products ranged from C₁ to C₁₇ and were mainly linear and mono-branched olefins in the C₂-C₈ range (FIG. 44C). In summary, solid acid materials HY, HZSM-5, and Al-SBA-15 are significantly more active than hydrocracking catalysts in converting additive-containing polymers to an alkene, an alkane, or a mixture thereof.

### Example 8. Technoeconomic Viability and Greenhouse Gas Emissions

Technoeconomic analysis to produce lubricants from olefins, where the minimum selling price can be compared readily with available market values, estimates a minimum selling price using Al-SBA-11 of $5.30/gal vs. the maximum selling price of API Grade I lubricants over the last decade of $5.88/gal (See Synlube, Base Oil Prices in USA. Available in: http://www.synlube.com/BaseOilPrices.html (20th April) (2022)) for a 32,000 Mt/y of LDPE with a price of $0.44/kg (See The Recycler's Exchange, Clear LDPE Scrap (loose), USA BRITISH COLUMBIA Available in: https://www.recycle.net/Plastic/ldpe/xv100400.html (20th April) (2022)). Higher grader poly-alpha olefin base-oils have much higher prices and can be produced via oligomerization. It was estimated that CO₂ emissions of 1.07 kg_{CO2}/kg_{lubricant}, representing an 8% reduction from the current oil-based route (See F. Brunner, Base oil production, petroleum refinery operation—RoW—base oil, Ecoinvent 3.8 Dataset (2021)). This emissions reduction is modest with the current electricity mix in the US due to the high contribution of fossil-based sources. MWs from renewable sources could reduce the emissions by 0.57 kg_{CO2}/kg_{lubricant}, a 51% reduction than oil-based lubricants. A more detailed description and sensitivity analyses are provided below.

### Process Modelling

Pure LDPE is fed into the pyrolysis reactor (FIG. 10). The experimental yields are used. Mass and energy balances are performed in Aspen Plus®. The energy requirements obtained in Aspen Plus simulation are corrected offline with the energy efficiency of the power supply, ϵ_{supply}=95%, and the energy efficiency of the magnetron, ϵ_{mag}89% (See J. M. Serra, etc., Hydrogen production via microwave-induced water splitting at low temperature, Nat Energy 5(11) (2020) 910-919), see the following equation. Heat losses due to convection are not considered.

$${E}_{r e q} = \frac{Δ {H}_{reactor}}{{ε}_{m a g} {ε}_{supply}}$$

When conventionally heated reactors are used, a pyrolysis chamber fuelled with natural gas and heating efficiency of 90% is considered (See HeatMatrix, Increase fired heater efficiency up to 95%, Available in: https://heatmatrixgroup.com/thermal-processes/fired-heater/#:˜:text=Fired%20Heaters%20are%20used%20in,around%2085%25%20%E2%80%93%2090%25. (2021)). The liquid and solids obtained from pyrolysis are sent to a filter to remove the unreacted LDPE. The liquid is then mixed with the gases and cooled down for separating the liquid olefins.

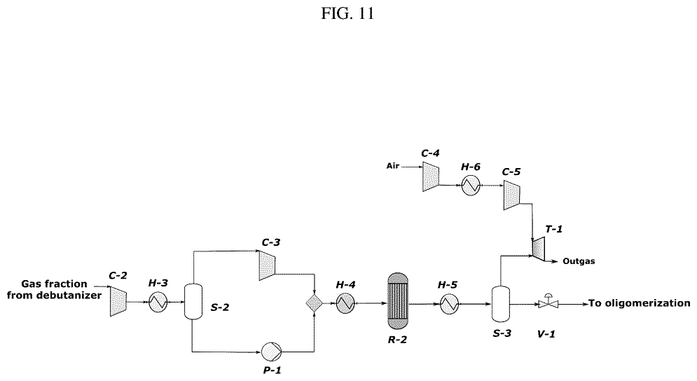

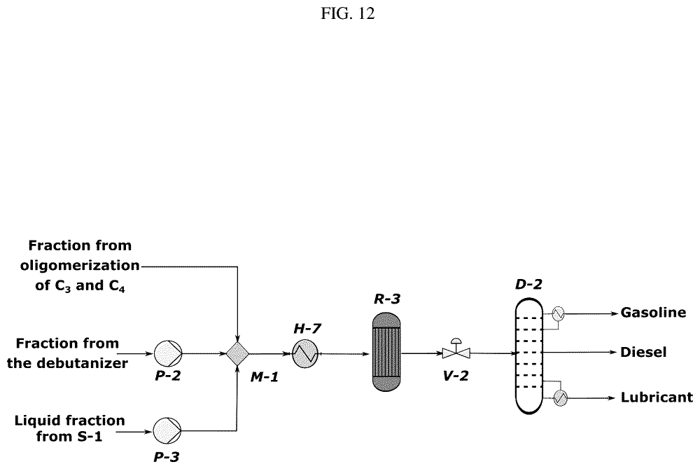

The presence of nitrogen reduces the efficiency of separation and high fractions, up to C8, are obtained in the gas stream at 1 atm. This gas stream is sent to a debutanizer, D-1 in FIG. 10, to recover the fractions higher than C4. The liquid product of the debutanizer is mixed with the liquid stream of S-1 and sent to the oligomerization process in FIG. 12. The distillate of the debutanizer is mainly composed of C3 and C4 fractions, and it is sent to an oligomerization reactor for light olefins, see FIG. 11. Oligomerization of light olefins with HZSM-5 as a catalyst in reactor R-2 takes place at 70 bar and 473 K with 98% of conversion (See C. S. Hsia-Chean, etc., Production of lubricant range hydrocarbons from light olefins, U.S. Pat. No. 4,568,786 (709143) (1986)). The high pressure required is achieved using two compressors with an intermediate cooling step. Two steps are required due to the limitations of the compressors in the pressure ratio and operating temperature. The intermediate cooling step results in the generation of a liquid fraction that cannot be sent to the compressors since it damages them. Thus, a pump is used to bypass the liquid fraction. The final gas-liquid mixture at 70 bar is heated to 473 K before being introduced into reactor R-2. As a result of oligomerization, the pressure at the exit is reduced to 17 bar. At these conditions, part of the C8 olefin fraction obtained from oligomerization is in the gas phase, so it needs to be cooled down to minimize the losses of the α-olefins generated in the flash separator and avoid the C4 and C3 paraffins to be obtained in the liquid. The liquid phase requires reducing the pressure before being sent to the oligomerization reactor of α-olefins, which operates at 15 bar. The gas phase obtained from the flash separator is sent to a gas turbine with an integrated combustion chamber, where it is burnt to produce power. The gas turbine operates with a pressure ratio of 16:1, and it requires air with an excess of 300% of the stoichiometric one to avoid extreme temperatures that damage the blades.

This liquid fraction obtained from the oligomerization of C2-C4 olefins is mixed with the liquid streams of the debutanizer and flash separator S-1. Oligomerization of α-olefins with HZSM-5 achieves a conversion of 92% and takes place at 423 K so that a heat exchanger is placed before the mixture. The product composed of lubricants, unreacted olefins, and paraffins (assumed as inert) is then depressurized to 1 bar before being introduced into a fractional tower. The fractional tower is modelled using a Petrofrac model, and it has three streams leaving the tower: lubricants, diesel and gasoline fractions. The reflux ratio and number of trays between the product streams are designed following short-cut methods for separating C11 from C12(fraction that separates gasoline from diesel) and C19 from C20 (fraction assumed for separating the diesel from lubricants).

### Techno-Economic and Life Cycle Analyses

### Assumptions in the TEA

The minimum selling price (MSP) of lubricants is used for evaluating the economic feasibility of this technology. A recovery period of 10 years is assumed for the plant, and a corporate tax of 21% is also imposed on the profits. The estimation of the MSP requires computing the capital costs (CAPEX) and operating costs (OPEX) of the process. The Aspen Process Economic Analyzer v.11 is used to estimate the investment cost and the installation of all the units of the process except of the MW slurry reactor and the oligomerization reactors. All the costs estimated by Aspen Process Economic Analyzer v.11 are based on 2018 Q1, and thus, they are updated with the plant cost index of the Chemical Engineering Magazine to the values of 2021.

The capital cost of the MW slurry reactor is determined as the sum of the MW generator and the reactor as presented in the following equation:

$$Cost MW reactor = Cost M W generator + Cost reactor$$

$$Cost MW reactor = ( {m}_{LDPE}^{\cdot } \cdot {E}_{r e q} \cdot {C}_{M W} + {m}_{LDPE}^{\cdot } \cdot {t}_{res} \cdot \frac{{m}_{c a t}}{{m}_{L D P E}} ) \cdot 2 \cdot IF$$

The cost of the MW generator, C_{MW}, is a function of the power requirements as reported in J. M. Serra, et al., Hydrogen production via microwave-induced water splitting at low temperature, Nat Energy 5(11) (2020) 910-919. As a base case, the average value reported for centralized plants, $550/KW, is taken; a sensitivity analysis is also performed for the range of costs reported. The cost of the reactor is assumed to be the catalyst bed as in J. M. Serra above. The bed is composed by the zeolite and catalyst as defined in the materials section of this supplementary material. The price of the catalyst is estimated using the CatCost Tool of the U.S. Department of Energy. The Step Method available in the tool is used for estimating the catalyst cost. In all the cases, a medium size plant of 10 Mt/d with 1 day of production and 1 day of cleaning is used based on the default version. The remaining economic inputs are also using the default values. The following assumptions are considered for 0.5 Pt-HY(30) catalyst:
0.5 Pt-HY(30) catalyst uses the available process for Zeolites with Metal Active Sites. The materials used for generating the active site, H₂PtCl₆, is not available in the database so its price is obtained from industrial vendors as $100/kg.
The amount required of H₂PtCl₆ per kg of zeolite is determined based on the mass percentage of Pt in the catalyst and the molecular weight of Platinum and H₂PtCl₆ as in the following equation.

$${m}_{H 2 PtCl 6} = \frac{{m}_{P t}}{{m}_{zeolit}} \cdot \frac{{M}_{WH 2 PtCl 6}}{{M}_{W P t}}$$

The mass of HY-zeolite, used as support, requires 20% of alumina and 80% of silica based on the average values of the following patent for Y-type zeolite: U.S. Pat. No. 5,785,944.
Based on these inputs, the price is obtained to be $33.46/kg.

For the estimation of the cost of Al-SBA-15, the following assumptions are considered in using the CatCost tool:
AlCl₃ is used for the active sites following the concentration given above.
Since SBA-15 or its precursors are not available in the database of the tool, its price is assumed to be $400/kg. Sensitivity analysis has been performed in the range of $60/kg to $400/kg.
The solvent, ethanol, is not considered in the cost and it is assumed to be completely recycled in the process.
The process includes the units presented in Table 5, based on the description given above.
The price obtained for base case, conservative price of SBA-15 of $400/kg is $423.63/kg.

TABLE 5
Inputs in the CatCost for 0.5 Pt-HY(30).

| Type of unit | Number of units |
|---|---|
| Reactor, simple (mixing) | 1 |
| Kiln continuous direct (300-1290° C.) | 2 |
| Filter, rotary vacuum | 1 |
| Dryer rotary | 1 |

The cost obtained for the reactor and the MW generator is multiplied by 2 since the MW slurry reactor works in semi-batch and it was assumed that the time for reloading and regenerating the catalyst is half of the total. Furthermore, a conservative installation factor, IF, with a value of 2.5, is used.

The capital cost of the oligomerization reactors is determined by the cost of the catalyst plus the cost of the shell and tube unit. They are designed as fixed-bed reactors with the WHSV reported in the patent U.S. Pat. No. 4,568,786, and the LHSV reported in J. F. Knifton, etc., Olefin Oligomerization Via Zeolite Catalysis, Catal Lett 28(2-4) (1994) 223-230, used for designing the process. To ensure the same flow conditions as in the references, the flow rate used as a basis in the design is the total flow rate fed in the reactor. The amount of HZSM-5 required in the reactor is computed as presented in the following equation.

$${M}_{zeolite} = \frac{\dot{F}}{W H S V} = \frac{\dot{V}}{L H S V} \cdot {ρ}_{z e o}$$

In the case of oligomerization of α-olefins (a LHSV is provided), the mass of HZSM-5 is obtained from the volume of zeolite required and using a zeolite density of 2,300 kg/m³. The price for HZSM-5 has been determined using the CatCost tool with the same economic inputs than for previous catalysts. The process used for the estimation is the one available in the tool for ZSM-5 zeolites. The materials used are 50% alumina bulk and 50% sodium silicate based on the patent U.S. Pat. No. 4,139,600. The price obtained is $9.72/kg, which is similar to the one of commercial vendors online, $10/kg (See Jiangxi Xiantao Technology Corporation, Molecular sieve Zeolite ZSM 5 for Petroleum Industry (20th April) (2022)).

The volume of the reactor is computed from the mass needed for HZSM-5 and the bulk density of HZSM-5 zeolite pellets, 720 kg/m³. The estimated volume is used for designing the reactor as a shell and tube unit. Multiple tubes of 1″ and a length of 20 feet (a standard size in shell and tube heat exchangers) are used in parallel. The number of tubes is determined by dividing the total volume needed by the volume of every tube. The total number of tubes is finally used for estimating the cost of the shell and tube unit as a TEMA heat exchanger in Aspen Economic Analyzer v.11. The cost reported by Aspen Economic Analyzer for the vessel is updated to 2021 with the plant cost index of the Chemical Engineering Magazine.

The remaining capital expenses (control equipment, piping, electrical installation, building, yard, service, land, engineering cost of the project, construction expenses, contractor's fee, and contingency) for the estimation of the total CAPEX are computed using the average value of the factors reported in M. Peters, etc., Plant Design and Economics for Chemical Engineers, 5th ed.2002.

The OPEX is estimated including the following:
The cost of the raw materials. Pure LDPE is considered at a price of $0.44/kg. The nitrogen carried gas was taken at a price of 0.177$CAD/m³, or $0.14/m³.
The cost of the utilities. The electricity price is taken as ¢7.3/kWh. The price for natural gas used in heating is assumed to be $4.38/MMBTU. The price of refrigerating water is taken from Aspen Plus, 2.1·10⁻⁷ $/kJ.
The labor cost of the operators. The number of operators, 21, each of them working 8 h per day, is determined as a function of the shifts in the plant following the recommendations given in. The wage required per operator is $70,200/y.
The cost of the supervision is computed as 15% of the total labor cost of the operators.
Maintenance cost per year is determined using a value of 4% of the total CAPEX.
Operating charges are computed as the sum of the charges for product control in the laboratories and the cost of insurance, local property taxes, rent, etc. Each of these terms is estimated with an average value of 15% of the labor expenses.
Plant overhead costs are determined as 60% of the total expense for operating labor, supervision, and maintenance.
Administrative costs are computed as 25% of the total labor costs.
The co-products obtained, gasoline and diesel, are assumed to provide a discount on the estimated costs. The selling price taken (without the taxes and distribution) is $3.31/gal for gasoline and $3.64/gal for diesel.

The results for the MSPs for lubricants using the different cases of Table 6 are reported in Table 7 and compared with the price of API Grade I lubricants during the last decade.

TABLE 6
Cases evaluated in the TEA and LCA studies.

| Case Study | Description of the technology |
|---|---|
| Conv | LDPE pyrolysis with conventional heating. |
| Monolith-Pt | LDPE pyrolysis with conventional heating |
| | and a monolith with 0.5Pt-HY(30) for |
| | improving the heat transfer. |
| Monolith-Al | LDPE pyrolysis with conventional heating |
| | and a monolith with Al-SBA-15 for improving |
| | the selectivity to olefins and the heat transfer. |
| MW-Pt | LDPE MW pyrolysis on 0.5Pt-HY(30). |
| MW-Al | LDPE MW pyrolysis on Al-SBA-15. |

TABLE 7
Minimum selling price and CAPEX of the facilities
to be built in each of the case studies.

| | MSP lubricant | MSP lubricant^{a} | CAPEX |
|---|---|---|---|
| Case Study | ($/gal) | ($/kg) | (MM$) |
| Conv | 352.48 | 109.42 | 30.9 |
| Monolith-Pt | 26.60 | 8.26 | 35.6 |
| Monolith-Al | 6.14 | 1.91 | 37.9 |
| MW-Pt | 8.86 | 2.75 | 65.5 |
| MW-Al | 5.30 | 1.64 | 66.2 |
| Oil-based lubricants | 5.88-1.94 | | |

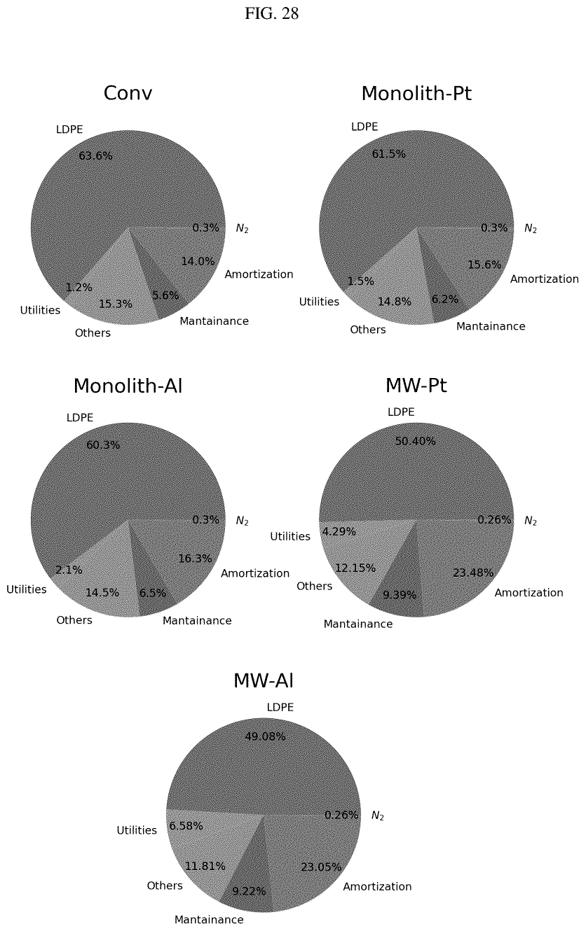

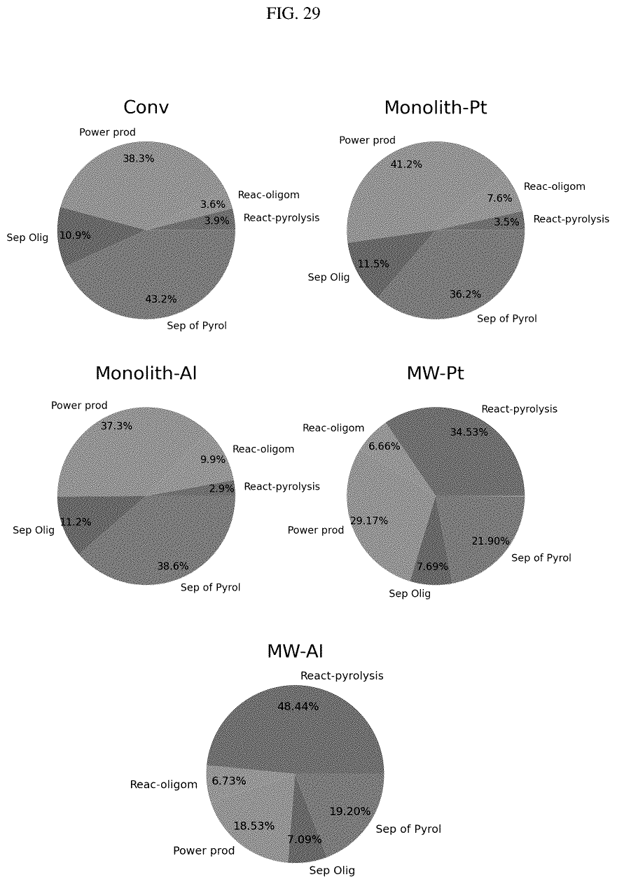

The distribution of the costs per technology is given in FIG. 28, and the breakdown of the CAPEX required in the units is provided in FIG. 29.

### Sensitivity Analysis in the TEA

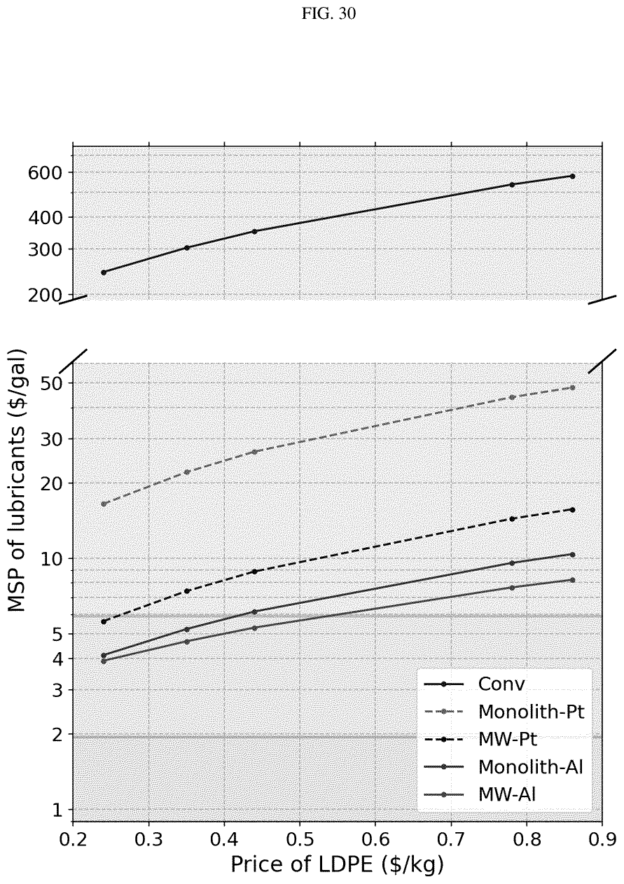

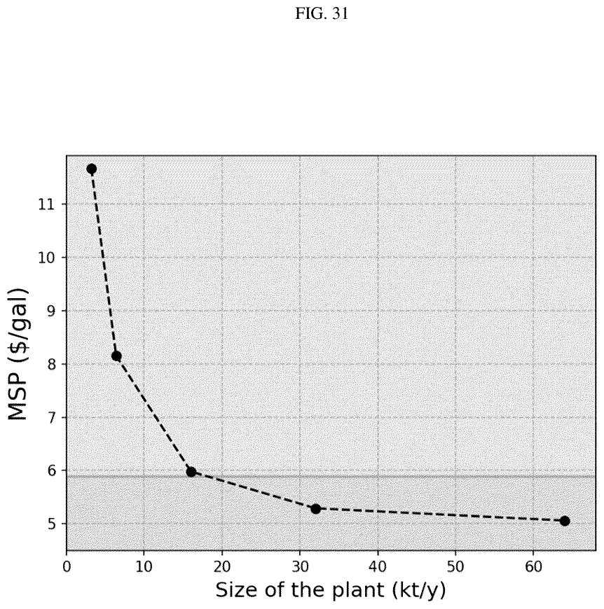

The evaluation of the costs was augmented with a sensitivity analysis of the different terms with the aim of evaluating alternative scenarios. The following components and ranges have been considered in the evaluation:
The effect of the price of LDPE on the MSP for all the case studies of Table 6. A wide range of prices is reported by potential suppliers ($0.24/kg in Greece, $0.35/kg in Ohio, and $0.77/kg in Florida) and an upper value of $0.85/kg. The results obtained for the different LDPE prices are plotted in FIG. 30.
Since Case MW-Al is determined as the most profitable alternative obtained, the following sensitivity analyses are performed:
Different scales. A minimum scale is fixed to be one tenth of the current plant, 3.2 k Mt/y, which is slightly smaller than two current operating pyrolysis plants of plastic pyrolysis in Europe with a size of 5 k Mt/y. A maximum is fixed to be double of the current plant, 64 Mt/y, which is 2% of the total LDPE in US and it is 3 times the size of the largest plant in Europe, 20 k Mt/y, and double the size of a plant that ExxonMobil is constructing with Plastic Energy in the Notre Dame de Gravenchon petrochemical complex, 33 k Mt/y. Results are presented in Table 8 and FIG. 31.

TABLE 8
Effect of the scale on the CAPEX and MSP of
the lubricants using MW-Al as technology.

| Size | MSP of lubricant | CAPEX |
|---|---|---|
| (k Mt/y) | ($/gal) | (MM$) |
| 64 | 5.06 | 113.8 |
| 32 (base case) | 5.29 | 66.2 |
| 16 | 5.98 | 44.3 |
| 6.4 | 8.15 | 31.4 |
| 3.2 | 11.67 | 27.8 |

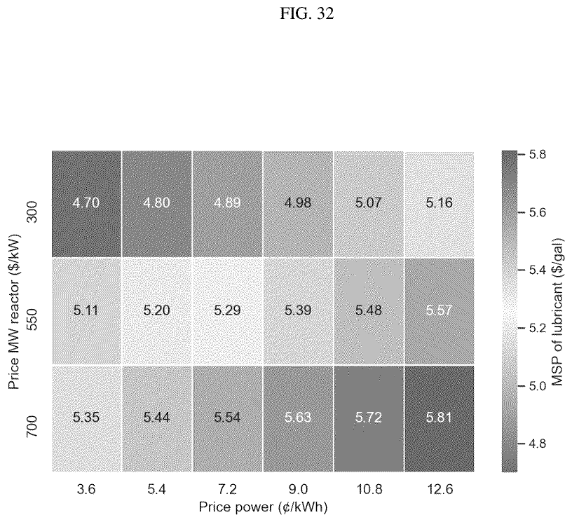

The costs involved in the MW slurry reactor are also analyzed. These costs correspond to the investment cost required for the reactor, which is the biggest contributor to the capital cost and the cost of electricity used for pyrolyzing the LDPE. The costs for the MW reactor are evaluated for the ranges provided in J. M. Serra above. The upper value corresponds to the maximum value reported for centralized plants, and the lowest value to the minimum expected in the future. The cost of electricity is evaluated assuming a maximum price of electricity for residential consumers, ¢12.6/kWh, and a minimum of half of the current price. The results are presented in FIG. 32.
The effect of the price of SBA-15 on the price of the catalyst and the MSP of the lubricants. SBA-15 has been studied over a range of prices given in “Zr Catalyst Co., Sba-15 catalyst mesoporous silica pore size sba-15 zeolite.” Available in: https://www.alibaba.com/product-detail/sba-15-catalyst-mesoporous-silica-pore_1600225434897.html?spm=a2700.galleryofferlist.normal_offer.d_title.67d0441f Uxrpk2 (2022), obtaining the results of Table 9.

TABLE 9
Effect of Installation Factor of the MW reactor.

| | MSP of lubricant | CAPEX |
|---|---|---|
| Installation Factor | ($/gal) | (MM$) |
| 1.1 | 4.80 | 49.7 |
| 1.5 | 4.93 | 54.4 |
| 2 | 5.11 | 60.3 |
| 2.5 (base case) | 5.29 | 66.2 |

The effect of the installation factor. A conservative scenario has been assumed as a base-case with a value of 2.5. However, lower installation factors, up to 1.1 can be considered based on the ranges reported in J. R. Couper, etc., Chemical Process Equipment Selection and Design, (2010). The results of the study are provided in Table 10.

TABLE 10
Effect of the price of SBA-15 on the catalyst,
the CAPEX and the MSP of the lubricants.

| | Price | MSP | Cost MW reactor | |
|---|---|---|---|---|
| Price SBA- | Catalyst | lubricants | with installation | CAPEX total |
| 15 ($/kg) | ($/kg) | ($/gal) | (MM$) | (MM$) |
| 60 | 63.58 | 5.229 | 8.98 | 64 |
| 100 | 105.94 | 5.237 | 9.06 | 64.2 |
| 200 | 211.84 | 5.257 | 9.25 | 64.9 |
| 300 | 317.73 | 5.277 | 9.45 | 65.5 |
| 400 | 423.63 | 5.297 | 9.65 | 66.2 |

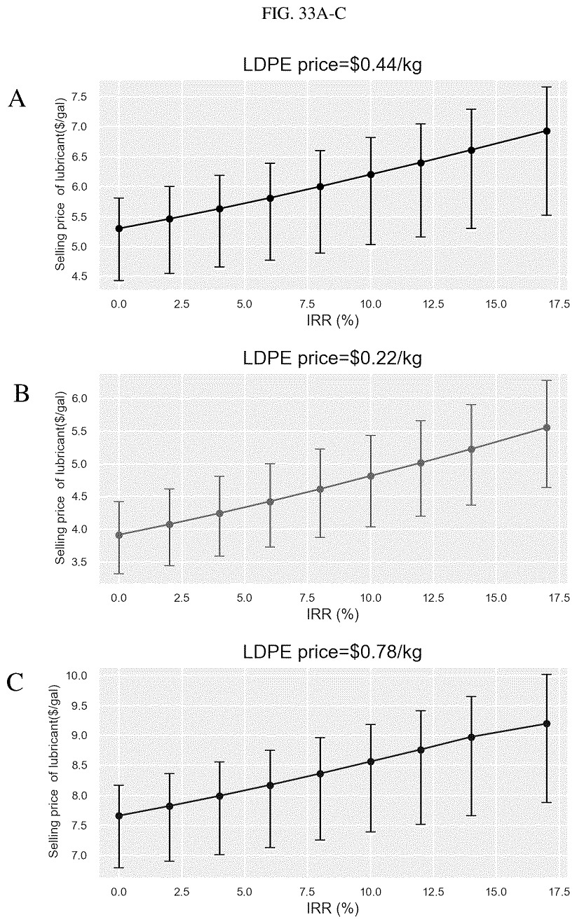

Price of the lubricants, above the MSP, based on the Internal Rate of Return (IRR) desired by the investor of the project. The base case has been studied for estimating the MSP, without considering any earnings. However, the investor would expect a benefit and prices above the MSP would be required. A sensitivity analysis has been performed for evaluating the selling price of the lubricants as a function of the desired IRR. The range evaluated goes from zero up to the highest value reported for specialty chemicals, 17%. The results, under different LDPE prices, are given in FIG. 33A-C.

### Assumptions in the LCA

The following assumptions are considered for the LCA:
The materials in continuous manufacturing are considered. Other materials used in construction, infrastructure, and catalysts are supposed to be recycled at the end of the process' life cycle and are not considered.
LDPE to be recycled is taken, and it is assumed to be provided by an external vendor located 100 km away from the plant.
The unconverted LDPE and the coke are considered wastes, and they are sent to a third-party company that treats them.
Flue gas from the process contains only CO₂. Other possible compounds (e.g., NO₂) are not considered.
Cooling water is assumed to have a gradient of 5° C. This supposes that 1% of the total cooling water is emitted into the atmosphere meanwhile the remaining 99% is recycled.
Electricity is supplied from a medium voltage grid based on the average technology and loss in the US.
Natural gas is assumed to be supplied at high pressure from a third vendor.
Gasoline and diesel are assumed to replace unleaded gasoline and diesel from petrol sources.

The environmental impacts have been calculated via the Traci method using Ecoinvent v 3.8as a database. The results for the case studies defined in Table 2 are presented in Table 11.

TABLE 11
Results of the LCA for each case study with system expansion method
and emissions generated in the production of lubricants from oil.

| | Case study | | | | | |
|---|---|---|---|---|---|---|
| Indicator^{a} | Conv | Monolith-Al | Monolith-Pt | MW-Pt | MW-Al | Oil-based |
| Acid | 6.957 | 0.040 | −0.293 | −0.271 | 0.063 | 0.490 |
| Ecotox. | 27.04 | 0.445 | 1.825 | 0.441 | 0.329 | 0.228 |
| Eutroph | 0.786 | 0.014 | 0.070 | 0.021 | 0.010 | 1.37 · 10⁻³ |
| GW | 60.85 | 1.113 | 4.124 | 2.319 | 1.068 | 1.170 |
| Ozone Dep. | 2.9 · 10⁻⁷ | −1.9 · 10⁻⁷ | −2.0 · 10⁻⁶ | −1.3 · 10⁻⁶ | −1.7 · 10⁻⁷ | 7.92 · 10⁻⁷ |
| Photox. | 0.0841 | 1.0 · 10⁻³ | 1.3 · 10⁻³ | −1.0 · 10⁻³ | 8.3 · 10⁻⁴ | 2.88 · 10⁻³ |
| Carc. | 0.358 | 0.006 | 0.030 | 0.010 | 4.9 · 10⁻³ | 1.64 · 10⁻³ |
| Non-Carc. | 627.1 | 12.715 | 53.653 | 16.263 | 9.312 | 6.209 |
| Resp.effects | 0.055 | 4.2 · 10⁻⁴ | −5.3 · 10⁻⁴ | 3.2 · 10⁻³ | 2.5 · 10⁻³ | 2.30 · 10⁻³ |

^{a}—Acid. Corresponds to the acidification potential in (mols of H⁺ Eq./kg_{lubricant}), Ecotox. Corresponds to the ecotoxicity potential in (kg 2,4-D Eq./kg_{lubricant}) Eutroph. Corresponds to the eutrophication potential in (kgN/kg_{lubricant}), GW corresponds to the grobal warming potential in (kg CO₂ Eq./kg_{lubricant}), Ozone Dep. Corresponds to the ozone deplention in (kg CFC-11-Eq/kg_{lubricant}), Photox. Corresponds to the photochemical oxidation potential in (kg NOₓ-Eq./kg_{lubricant}), Carc. Corresponds to the carcinogenic potential in (kg benzene-Eq/kg_{lubricant}), Non-Carc. Corresponds to the emissions of non-carcinogenic compounds in (kg toluene-Eq./kg_{lubricant}), Resp. effects corresponds to the respiratory effects in (kg PM_{2.5}Eq./kg_{lubricant}).

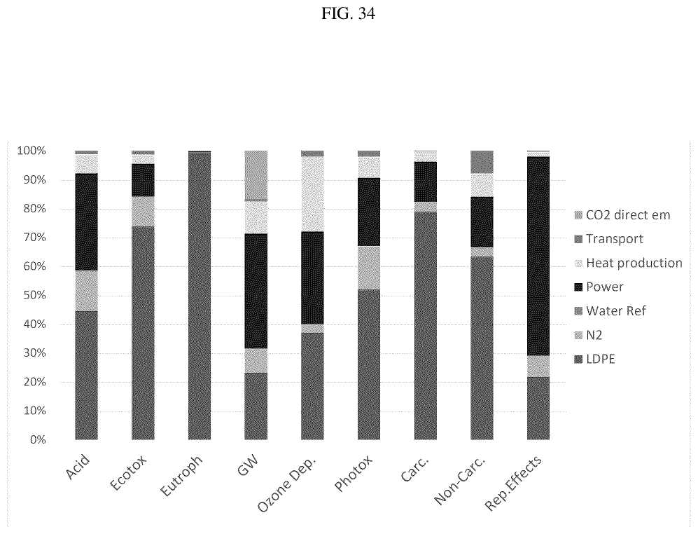

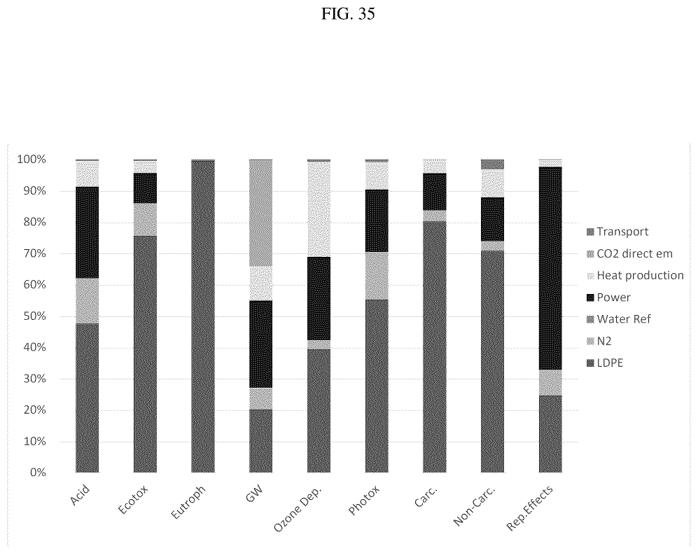

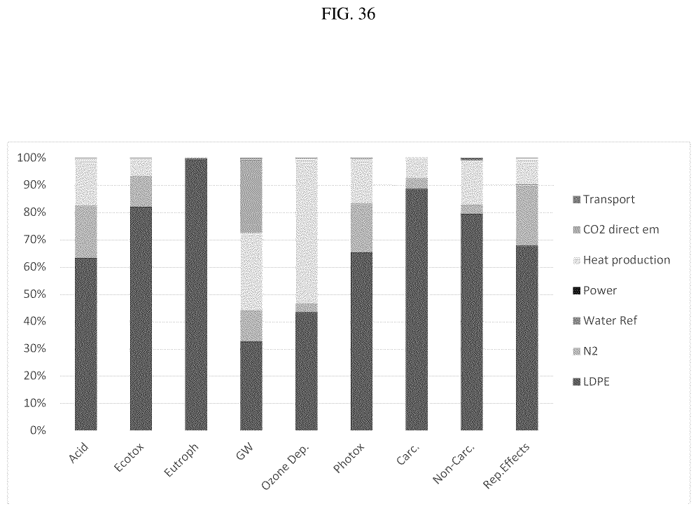

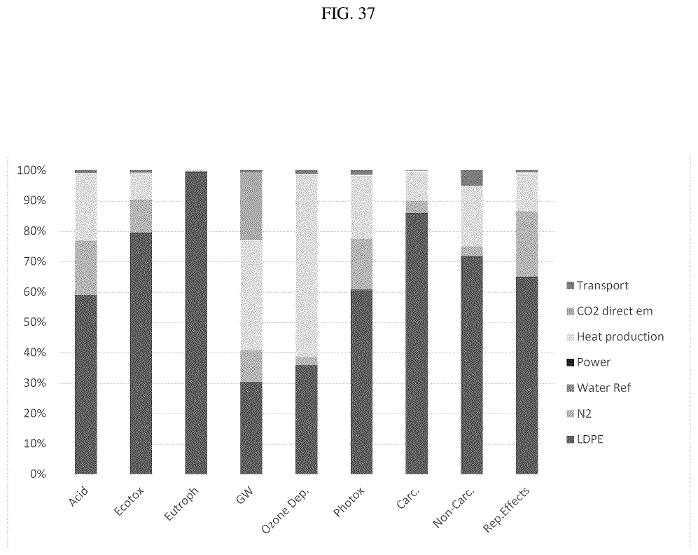

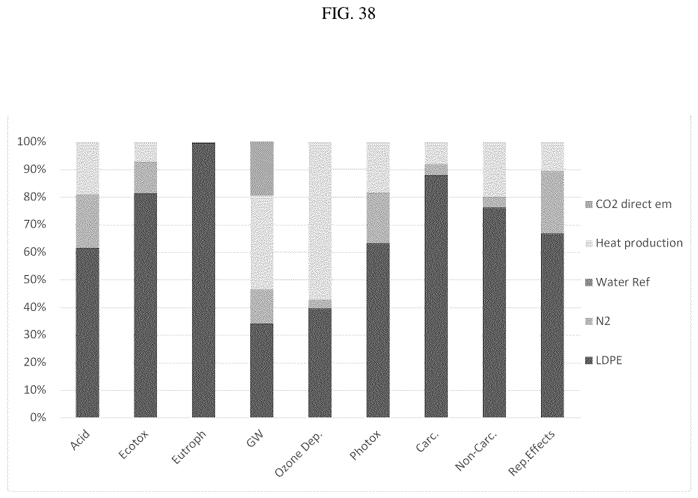

The distribution of the emissions between the different positive contributors are provided in FIG. 34 to FIG. 38 for each of the cases. The results obtained in Table 11 show that some cases have indicators with a negative value. These negative values are due to the credits obtained from the by-products, gasoline and diesel, that substitute current oil-based products with system expansion method. For a better understanding of the results, the credits obtained are given in Table 12.

TABLE 12
Credits obtained from fuels.

| | Case study | | | | |
|---|---|---|---|---|---|
| Indicator^{a} | Conv | Monolith-Al | Monolith-Pt | MW-Pt | MW-Al |
| Acid | 3.17 | 0.163 | 1.246 | 0.675 | 0.117 |
| Ecotox. | 2.49 | 0.128 | 0.971 | 0.527 | 0.091 |
| Eutroph | 0.011 | 5.5 · 10⁻⁴ | 4.32 · 10⁻³ | 2.437 · 10⁻³ | 4.31 · 10⁻⁴ |
| GW | 7.432 | 0.385 | 2.848 | 1.300 | 0.2243 |
| Ozone Dep. | 6.5 · 10⁻⁶ | 3.4 · 10⁻⁷ | 2.6 · 10⁻⁶ | 1.47 · 10⁻⁶ | 2.61 · 10⁻⁷ |
| Photox. | 0.0223 | 1.16 · 10⁻³ | 8.89 · 10⁻³ | 4.82 · 10⁻³ | 8.45 · 10⁻⁴ |
| Carc. | 0.0122 | 6.3 · 10⁻⁴ | 4.62 · 10⁻³ | 2.18 · 10⁻³ | 3.71 · 10⁻⁴ |
| Non-Carc. | 44.26 | 2.272 | 16.98 | 8.593 | 1.471 |
| Resp. effects | 0.020 | 1.05 · 10⁻³ | 7.62 · 10⁻³ | 3.02 · 10⁻³ | 5.23 · 10⁻⁴ |

It can be seen that the credits are mainly relevant in those cases with a higher paraffins-olefins ratio (Conv, Monolith-Pt and MW-Pt). Although case “Conv” shows the highest credits, it is not due to the avoidance of emissions. It is because the mass of lubricants produced is very low and all the emissions per kg of lubricant are very high. In the same way, it is also important to note that having higher credits does not ensure a lower impact since all the emissions are also divided by the mass of lubricants produced. This is particularly significant in the Global Warming Potential, where MW-Al with a higher production of lubricants shows a better performance than the other technologies; see Table 11.

### Sensitivity Analysis for the LCA

The most profitable and interesting technology due to its economic profitability is the MW-Al. Apart from the economic profitability due to the selectivity of the catalyst and the better distribution of the heat, the MW slurry reactor also has the potential of integrating energy supplied from a renewable source. A sensitivity analysis is performed assuming that the energy required in the process is obtained from two types of renewable sources: photovoltaic and wind energy sources. The results obtained are presented in Table 13.

TABLE 13
Results of the LCA for MW-Al with
conventional supply and renewable energy supply.

| | | | | Reduction of emissions compared | | |
|---|---|---|---|---|---|---|
| | Emissions generated | | | to oil-based lubricants in (%) | | |
| | Power based | Photovoltaic | Wind | Power based | Photovoltaic | Wind |
| Indicator^{a} | on US mix | energy | energy | on US mix | energy | energy |
| Acid | 0.063 | 0.022 | 6.74 · 10⁻³ | −87.1 | −95.5 | −98.6 |
| Ecotox. | 0.329 | 0.353 | 0.298 | 44.6 | 55.0 | 30.6 |
| Eutroph | 0.010 | 0.010 | 9.77 · 10⁻⁷ | 615.3 | 615.6 | 613.6 |
| GW | 1.068 | 0.627 | 0.570 | −8.7 | −46.4 | −51.2 |
| Ozone Dep. | −1.7 · 10⁻⁷ | −1.9 · 10⁻⁷ | −1.96 · 10⁻⁷ | −121.2 | −123.9 | −124.7 |
| Photox. | 8.3 · 10⁻⁴ | 6.2 · 10⁻⁴ | 4.801 · 10⁻⁴ | −71.1 | −78.4 | −83.3 |
| Carc. | 4.9 · 10⁻³ | 0.005 | 4.42 · 10⁻³ | 199.6 | 208.7 | 169.4 |
| Non-Carc. | 9.312 | 9.100 | 7.803 | 50.0 | 46.6 | 25.7 |
| Resp.effects | 2.5 · 10⁻⁷ | 5.4 · 10⁻⁴ | 4.30 · 10⁻⁴ | 6.7 | −76.7 | −81.3 |

Most of the indicators show a reduction of the emissions when any of the renewable technologies are substituted by the current electricity mix of US. This particularly relevant in the Global Warming potential, showing a reduction of nearly 50% in the CO₂ emissions.

The emissions obtained with the current processes are compared with the results reported for oil-based lubricants. The difference between them is computed with the percentages defined by the following equation and the results are provided in Table 14.

$$\% reduction of the emissions = \frac{{Factor}_{process} - {Factor}_{oil}}{{Factor}_{oil}} \cdot 100$$

TABLE 14
Comparison of emissions versus oil-based lubricants.

| | Reduction of emissions compared to oil-based | | | | |
|---|---|---|---|---|---|
| | in (%) for different cases | | | | |
| Indicator^{a} | Conv | Monolith-Al | Monolith-Pt | MW-Pt | MW-Al |
| Acid | 1319.5 | −91.9 | −159.9 | −155.2 | −87.1 |
| Ecotox. | 11773.3 | 95.4 | 701.2 | 93.6 | 44.6 |
| Eutroph | 57296.5 | 945.0 | 5007.2 | 1462.0 | 615.3 |
| GW | 5101.5 | −4.8 | 252.5 | 98.2 | −8.7 |
| Ozone Dep. | −63.6 | −123.7 | −352.9 | −258.3 | −121.2 |
| Photox. | 2818.1 | −65.4 | −56.3 | −134.8 | −71.1 |
| Carc. | 21682.6 | 292.3 | 1711.8 | 507.2 | 199.6 |
| Non-Carc. | 10000.5 | 104.8 | 764.1 | 161.9 | 50.0 |
| Resp. effects | 2308.9 | −81.6 | −123.1 | 39.8 | 6.7 |

### Sensitivity Analysis to the Type of Method Used in the LCA

For further comparison, the mass allocation and economic allocation methods have also been used for evaluating the environmental indicators. The results obtained for the former are given in Table 15 and the reduction with respect to oil-based lubricants is provided in Table 16. The results for the economic allocation method are reported in Table 17 and Table 18.

TABLE 15
Environmental indicators obtained using the mass allocation method.

| | Case study | | | | | |
|---|---|---|---|---|---|---|
| Indicator^{a} | Conv | Monolith-Al | Monolith-Pt | MW-Pt | MW-AI | Oil-based |
| Acid | 0.089 | 0.085 | 0.037 | 0.039 | 0.093 | 0.490 |
| Ecotox. | 0.264 | 0.252 | 0.115 | 0.094 | 0.217 | 0.228 |
| Eutroph | 7.17 · 10⁻³ | 0.007 | 0.003 | 0.002 | 0.005 | 1.37 · 10⁻³ |
| GW | 0.600 | 0.628 | 0.267 | 0.351 | 0.666 | 1.170 |
| Ozone Dep. | 5.99 · 10⁻⁸ | 6.4 · 10⁻⁸ | 2.4 · 10⁻⁸ | 2.1 · 10⁻⁸ | 4.8 · 10⁻⁸ | 7.92 · 10⁻⁷ |
| Photox. | 0.00094 | 9.2 · 10⁻⁴ | 4.0 · 10⁻⁴ | 3.7 · 10⁻⁴ | 8.5 · 10⁻⁴ | 2.88 · 10⁻³ |
| Carc. | 3.31 · 10⁻³ | 0.003 | 0.001 | 0.001 | 2.7 · 10⁻³ | 1.64 · 10⁻³ |
| Non-Carc. | 5.998 | 6.545 | 2.865 | 2.411 | 5.562 | 6.209 |
| Resp.effects | 6.2 · 10⁻⁴ | 4.9 · 10⁻⁴ | 2.0 · 10⁻⁴ | 6.1 · 10⁻⁴ | 1.5 · 10⁻³ | 2.30 · 10⁻³ |

TABLE 16
Comparison of potentials versus oil-based
lubricants using the mass allocation method.

| | Reduction of emissions compared to oil-based | | | | |
|---|---|---|---|---|---|
| | lubricants in (%) for different cases | | | | |
| Indicator^{a} | Conv | Monolith-Al | Monolith-Pt | MW-Pt | MW-AI |
| Acid | −81.73 | −82.62 | −92.53 | −92.04 | −81.11 |
| Ecotox. | 16.27 | 10.62 | −49.71 | −58.76 | −4.86 |
| Eutroph | 424.24 | 384.16 | 126.52 | 68.74 | 285.14 |
| GW | −48.68 | −46.33 | −77.21 | −70.01 | −43.04 |
| Ozone Dep. | −92.44 | −91.93 | −96.91 | −97.34 | −93.94 |
| Photox. | −67.10 | −68.22 | −86.18 | −87.32 | −70.43 |
| Carc. | 101.70 | 88.59 | −14.67 | −28.20 | 66.19 |
| Non-Carc. | −3.40 | 5.41 | −53.86 | −61.17 | −10.42 |
| Resp.effects | −73.07 | −78.61 | −91.44 | −73.73 | −33.28 |

TABLE 17
Environmental indicators obtained using the economic allocation method.

| | Case study | | | | | |
|---|---|---|---|---|---|---|
| Indicator^{a} | Conv | Monolith-Al | Monolith-Pt | MW-Pt | MW-Al | Oil-based |
| Acid | 0.137 | 0.977 | 0.053 | 0.053 | 0.1043 | 0.490 |
| Ecotox. | 0.404 | 0.289 | 0.166 | 0.1283 | 0.244 | 0.228 |
| Eutroph | 0.0109 | 7.607 · 10⁻³ | 4.511E · 10⁻³ | 3.156 · 10⁻³ | 5.94 · 10⁻³ | 1.37 · 10⁻³ |
| GW | 0.916 | 0.7205 | 0.3878 | 0.479 | 0.750 | 1.170 |
| Ozone Dep. | 9.145 · 10⁻⁸ | 7.341 · 10⁻⁸ | 3.558E · 10⁻⁸ | 2.877 · 10⁻⁸ | 5.41 · 10⁻⁸ | 7.92 · 10⁻⁷ |
| Photox. | 1.449 · 10⁻³ | 1.052 · 10⁻³ | 5.795 · 10⁻⁴ | 4.994 · 10⁻⁴ | 9.6E−04 | 2.88 · 10⁻³ |
| Carc. | 5.058 · 10⁻³ | 3.553 · 10⁻³ | 2.038 · 10⁻³ | 1.610 · 10⁻³ | 3.07 · 10⁻³ | 1.64 · 10⁻³ |
| Non-Carc. | 9.160 | 7.509 | 4.166 | 3.293 | 6.263 | 6.209 |
| Resp.effects | 9.475 · 10⁻⁴ | 5.656 · 10⁻⁴ | 2.869 · 10⁻⁴ | 8.268 · 10⁻⁴ | 1.73 · 10⁻³ | 2.30 · 10⁻³ |

TABLE 18
Comparison of potentials versus oil-based lubricants
using the economic allocation method.

| | Reduction of emissions compared to oil-based | | | | |
|---|---|---|---|---|---|
| | lubricants in (%) for different cases | | | | |
| Indicator^{a} | Conv | Monolith-Al | Monolith-Pt | MW-Pt | MW-AI |
| Acid | −72.10 | −80.06 | −89.13 | −89.12 | −78.73 |
| Ecotox. | 77.57 | 26.92 | −26.86 | −43.67 | 7.13 |
| Eutroph | 700.64 | 455.52 | 229.43 | 130.50 | 333.67 |
| GW | −21.63 | −38.42 | −66.85 | −59.03 | −35.86 |
| Ozone Dep. | −88.46 | −90.74 | −95.51 | −96.37 | −93.18 |
| Photox. | −49.76 | −63.54 | −79.90 | −82.68 | −66.70 |
| Carc. | 208.04 | 116.38 | 24.10 | −1.93 | 87.14 |
| Non-Carc. | 47.52 | 20.94 | −32.90 | −46.96 | 0.87 |
| Resp. effects | −58.88 | −75.45 | −87.55 | −64.12 | −24.87 |

In the economic allocation method, the prices defined in the TEA have been assumed for gasoline and diesel; and the average price of the range reported for lubricants, $3.91/gal, have been used. These two methods are compared with the emissions obtained from the system expansion method. In nearly one half of the indicators, and in particular in the global warming, the system expansion method shows higher emissions than the economic and mass allocation methods. Thus, in order to be conservative, and for avoiding co-allocation in multiproduct systems, the emissions reported for CO₂ in the manuscript corresponds to the system expansion method.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

What is claimed is:

1. A pyrolysis process for converting a plastic comprising a polyolefin polymer to an alkene, comprising contacting the plastic with a catalyst in a one-pot pyrolysis system at a temperature between about 350° C. and about 500° C.; wherein the catalyst comprises a solid acid; and wherein the one-pot pyrolysis system comprises a microwave-assisted slurry reactor.

2. A pyrolysis process for converting an additive-containing plastic comprising a polyolefin polymer to an alkene, an alkane, or a mixture thereof, comprising contacting the additive-containing plastic with a catalyst in a one-pot pyrolysis system at a temperature between about 350° C. and about 500° C.; wherein the catalyst comprises a solid acid selected from the group consisting of HY, HZSM-5, and Al-SBA-15; and wherein the one-pot pyrolysis system comprises a microwave-assisted slurry reactor.

3. The pyrolysis process of claim 2, wherein the additive-containing plastic comprises one or more additives, wherein the additive is an antioxidant, a light stabilizer, a ultraviolet (UV) light absorber, a filler, a slip agent, a lubricant additive, a stabilizer, or a mixture thereof.

4. The pyrolysis process of claim 3, wherein the antioxidant is butylated hydroxytoluene (BHT), 1,3,5-Tris(3,5-di-tert-butyl-4-hydroxybenzyl) (I-3114), Diphenyl Phosphite (SAO), tris(2,4-di-tert-butylphenyl) phosphite (SAO-C), or a mixture thereof.

5. The pyrolysis process of claim 3, wherein the antioxidant is Diphenyl Phosphite (SAO), tris(2,4-di-tert-butylphenyl) phosphite (SAO-C), or a mixture thereof.

6. The pyrolysis process of claim 3, wherein the light stabilizer is 2,2,6,6-Tetramethylpiperidine (HALS), bis(2,2,6,6-tetramethyl-4-piperidyl) sebacate (HALS-C), or a mixture thereof.

7. The pyrolysis process of claim 3, wherein the UV light absorber is 2-(2-Hydroxy-5-methylphenyl) benzotriazole (BT), 2-(2H-Benzotriazol-2-yl)-6-dodecyl-4-methylphenol (BT-C), or a mixture thereof.

8. The pyrolysis process of claim 3, wherein the lubricant additive is octadecanoic acid (L-C18A), 1-Octadecanol (L-C18OH), or a mixture thereof.

9. The pyrolysis process of claim 3, wherein the slip agent is oleamide (SA).

10. The pyrolysis process of claim 2, wherein the catalyst is HY.

11. The pyrolysis process of claim 2, wherein the catalyst is HZSM-5.

12. The pyrolysis process of claim 2, wherein the catalyst is Al-SBA-15.

13. The process of claim 2, wherein the pyrolysis process is conducted in a gas flow at a rate of about 100 ml/min.

14. The process of claim 2, wherein the temperature is about 370° C.

15. The process of claim 2, wherein the alkene is a mixture selected from the group consisting of C₄ alkenes, C₅ alkenes, C₇-C₁₂ alkenes, and a mixture thereof.

16. The process of claim 2, wherein the weight ratio between the plastic and the catalyst is about 10:1.

17. The process of claim 2, wherein the yield of the alkene is at least about 80%.

18. The process of claim 2, wherein the process further comprises separating resulting liquid pyrolysis products from resulting gaseous pyrolysis products through a condenser section.

19. The process of claim 2, wherein the microwave-assisted slurry reactor comprises a reaction vessel and a microwave source.

20. A one-pot pyrolysis system comprising
a microwave-assisted slurry reactor; and
a condenser section;
wherein the microwave-assisted slurry reactor comprises a reaction vessel and a microwave source.
